(19) United States
(12) Patent Application Publication
(10) Pub. No.: US 2011/0076298 A1
(43) Pub. Date: Mar. 31, 2011

(54) Soluble stabilized trimeric hiv env proteins and uses thereof

(76) Inventors: William C. Olson, Yorktown Heights, NY (US); Paul J. Maddon, Scarsdale, NY (US); Michael Franti, Cambridge, MA (US); Sai Prasad N. Iyer, East Elmhurst, NY (US); Sofija Andjelic, Nanuet, NY (US); John P. Moore, New York, NY (US); Simon Beddows, New York, NY (US)

(21) Appl. No.: 12/308,681

(22) PCT Filed: Jun. 19, 2007
(86) PCT No.: PCT/US07/14389

§ 371 Date: Jun. 11, 2010

Related U.S. Application Data
(60) Provisional application No. 60/815,233, filed on Jun. 19, 2006.
(60) Provisional application No. 60/815,248, filed on Jun. 19, 2006.

(51) Int. Cl. A61K 39/21; C12N 5/10; C12N 15/63; A61P 31/18; A61K 38/16; C07K 14/155; C07H 21/04; C07H 21/02

(52) U.S. Cl. 424/188.1; 435/325; 435/320.1; 514/3.8; 514/21.2; 530/350; 536/23.72

(57) ABSTRACT

This invention provides a protein comprising (a) a first polypeptide which comprises consecutive amino acids, the sequence of which corresponds to the sequence of a modified gp120 envelope polypeptide portion of a gp140 envelope of (i) an HIV-I KNH1144 isolate, or a quasi-species thereof, or (ii) an HIV-I 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of (i) the HIV-I KNH1144 isolate or such quasi-species thereof, or (ii) the HIV-I 5768.4 isolate or such quasi-species thereof. This invention also provides nucleic acids encoding such proteins, vectors, host cells and compositions thereof. Also provided are trimeric complexes (‘trimers’) of these proteins and methods of using such trimers to combat HIV-I infection.

This invention was made with support under United States Government Grant Nos. AI 45463, AI 36082, and AI 30030 from the National Institutes of Health, and the Henry M. Jackson Foundation for the Advancement of Military Medicine under Cooperative Agreement Number DAMD17-98-2-8007 between the Foundation and the U.S. Army Medical Research Acquisition Activity (USAMRAA). Accordingly, the United States Government has certain rights in the subject invention.

Throughout this application, certain publications are referenced. Full citations for these publications may be found immediately preceding the claims. The disclosures of these publications in their entireties are hereby incorporated by reference into this application in order to more fully describe the state of the art to which this invention relates.

### BACKGROUND OF THE INVENTION

The sequence diversity of its RNA genome, particularly in the gag and env genes, has led to the subdivision of human immunodeficiency virus type 1 (HIV-1) strains into distinct genetic subtypes, or clades. The genetic diversity of HIV-1 has also been increased by the appearance of circulating recombinant forms; individuals infected with multiple HIV-1 strains from different genetic subtypes generate and transmit mosaic progeny viruses. The largest number of incident and total HIV-1 infections, and the greatest genome sequence diversity, are now found in sub-Saharan Africa. Kenya is a typical East African example, with a median prevalence of 9.4% (6.6-14.3%) in 2002. The majority of circulating strains in Kenya are homogeneously subtype A (˜56%), with subtype C (˜2%), subtype D (˜2%) and recombinant viruses derived predominantly from subtype A sequences (˜39%) also present. Similar patterns of sequence diversity have been found in other epidemiological surveys carried out in Kenya, and in neighboring East African countries.

It is generally accepted that an effective HIV-1 vaccine should induce broadly neutralizing antibodies (NAb), with other immune effector responses optimally also being induced. To minimize the influence of sequence diversity, it is logical that candidate immunogens should be based on contemporary strains circulating within the vaccine trial site. An alternative approach is to base immunogens on consensus sequences. To date, with few exceptions, oligomeric Env subunit vaccine candidates have been based on subtype B sequences. It seems prudent to explore whether Env trimers based on other subtypes might be superior for induction of NAbs with activities both within and across subtypes.

The functional Env complex on the surface of virions and infected cells is a homotrimer of heterodimers incorporating three gp120 surface (SU) and gp41 trans-membrane (TM) subunits, although the exact conformation of this complex can only be inferred from partial crystallographic analysis and topological mapping using antibodies. Only a few MAbs with relatively broad neutralizing activity against primary isolates have been identified, although very many more are active against highly passaged, laboratory-adapted strains. Although most known NAbs are directed against the gp120 subunit of the Env complex, immunization with gp120 monomers has failed to elicit such antibodies in monkeys or humans. As a result, a common assumption is that the next generation of vaccine candidates should be based on oligomeric forms of Env.

Several strategies for making Env oligomers are currently being pursued, most of which are based on producing recombinant soluble trimeric gp140 proteins by truncating the gp41 ectodomain (gp41_{ECTO}) immediately prior to its membrane-spanning domain. If the gp120-gp41 cleavage site is endoproteolytically processed, the resulting gp140 trimers are labile, because the non-covalent interactions between the gp120 and gp41 subunits are weak. Hence, a common approach to making gp140 trimers has been to eliminate the cleavage site by mutagenesis. Uncleaved gp140 proteins are usually expressed as mixtures of oligomeric forms from which trimers can be purified by size exclusion chromatography (SEC). Such gp140s can be further modified by addition of trimer-stabilizing, or immune enhancing, motifs. Uncleaved Env proteins are antigenically distinct from cleaved ones, a factor that may or may not matter from the perspective of Env protein immunogenicity.

This approach to Env trimer design leaves the cleavage site intact, with the gp120 and gp41_{ECTO} subunits being covalently linked by an intermolecular disulfide bond (SOS gp140), with a further modification to gp41 (I559P) to improve trimer stability (SOSIP gp140). To date, these studies have predominantly been focused on the subtype B primary strain, HIV-1_{JR-FL}. Immunization of rabbits with SOSIP gp140 trimers from HIV-1_{JR-FL} can induce antibodies capable of neutralizing the homologous strain, although their breadth of activity is limited.

### SUMMARY OF THE INVENTION

Described herein are stable, cleaved, trimeric Env proteins based on a subtype A template for use as an immunogen. Soluble SOSIP gp140 expression constructs were generated from six homogenously subtype A env genes. The expression of these proteins was then assessed and it was determined that SOSIP gp140 protein lead to the production of trimers that could be purified from other oligomeric forms. The extent of Env cleavage with and without Furin co-expression was also determined, along with the stability of the purified trimers. The antigenic configuration of the SOSIP gp140 trimers was explored by determining their reactivity with monoclonal antibodies (MAbs), which was compared with the neutralization sensitivity of the corresponding Env-pseudotyped virus. Also described are stable, cleaved, trimeric Env proteins based on a subtype B template for use as an immunogen.

This invention provides a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:11 and SEQ ID NO:12, respectively, said modified gp120 envelope polypeptide portion comprising a cysteine at amino acid position 511 and said modified gp41 ectodomain polypeptide portion comprising a cysteine at amino acid position 617 and a proline at amino acid position 571, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 511 and the cysteine at amino acid position 617.

This invention also provides a trimeric complex comprising three monomers, each of which is a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:11 and SEQ ID NO:12, respectively, said modified gp120 envelope polypeptide portion comprising a cysteine at amino acid position 511 and said modified gp41 ectodomain polypeptide portion comprising a cysteine at amino acid position 617 and a proline at amino acid position 571, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 511 and the cysteine at amino acid position 617.

This invention further provides a nucleic acid encoding a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:11 and SEQ ID NO:12, respectively, said modified gp120 envelope polypeptide portion comprising a cysteine at amino acid position 511 and said modified gp41 ectodomain polypeptide portion comprising a cysteine at amino acid position 617 and a proline at amino acid position 571, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 511 and the cysteine at amino acid position 617.

This invention also provides a protein comprising (a) a first polypeptide which comprises consecutive amino acids, the sequence of which corresponds to the sequence of a modified gp120 envelope polypeptide portion of a gp140 envelope of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625.

This invention further provides a trimeric complex comprising three monomers, each of which is a protein comprising (a) a first polypeptide which comprises consecutive amino acids, the sequence of which corresponds to the sequence of a modified gp120 envelope polypeptide portion of a gp140 envelope of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625.

This invention further provides a nucleic acid encoding a protein comprising (a) a first polypeptide which comprises consecutive amino acids, the sequence of which corresponds to the sequence of a modified gp120 envelope polypeptide portion of a gp140 envelope of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625.

This invention also provides a vector comprising a nucleic acid of the invention. This invention further provides a host cell comprising such vector. This invention also provides a composition comprising a trimeric complex of the invention, and a pharmaceutically acceptable carrier.

This invention provides a method for eliciting an immune response against HIV-1 or an HIV-1 infected cell in a subject comprising administering to the subject an amount of a trimeric complex of the invention effective to elicit the immune response in the subject.

This invention also provides a method for preventing a subject from becoming infected with HIV-1, which comprises administering to the subject a prophylactically effective amount of a trimeric complex of the invention so as to thereby prevent the subject from becoming infected with HIV-1.

This invention further provides a method for reducing the likelihood of a subject becoming infected with HIV-1, which comprises administering to the subject an amount of a trimeric complex of the invention effective to reduce the likelihood of the subject becoming infected with HIV-1.

This invention also provides a method for delaying the onset of, or slowing the rate of progression of, an HIV-1-related disease in an HIV-1-infected subject which comprises administering to the subject an amount of a trimeric complex of the invention effective to delay the onset of, or slowing the rate of progression of, the HIV-1-related disease in the subject.

This invention provides a trimeric Env complex. The trimeric complex may also include a non-ionic detergent. The Env protein comprising the trimeric complex may be a Clade A or a Clade B HIV-1 isolate.

The invention also provides an isolated nucleic acid having the sequence as set forth in SEQ ID NO:13, which encodes a modified gp120 polypeptide portion and a modified gp41 ectodomain polypeptide portion of the gp140 envelope protein of an HIV-1 5768.4 isolate.

### BRIEF DESCRIPTIONS OF THE FIGURES

FIGS. 1A and 1B:

Cleavage and oligomer formation of subtype A gp140 proteins. The Env proteins were expressed on a pilot-scale by transient transfection of 293T cells and were not purified before analysis. (A) Oligomer formation in SOS gp140 proteins containing (plus sign) or lacking (minus sign) the additional trimer-stabilizing substitution, I571P, was assessed by BN-PAGE. The gel shown is representative of several obtained from several independent transfections. (B) SOSIP gp140 proteins were expressed in the presence (+F) or absence (−F) of the endoprotease Furin, and SOSIP gp140 proteins incorporating the hexa-Arg motif (R6) were expressed in the absence of Furin, and analyzed by SDS-PAGE. The amount of the gp120 cleavage fragment present was calculated as a percentage of the total amount of Env proteins (gp140+gp120). The values recorded under the lanes reflect the mean % cleavage determined from 3-6 independent transfection experiments, of which the one depicted is representative.

FIG. 2:

Env forms present in KNH1211 SOSIP gp140, KNH1144 SOSIP gp140 and KNH1144 SOS gp140 proteins. The fractions representing the trimer, dimer and monomer peaks that are eluted from SEC columns in volumes of 12 to 15 ml are shown.

FIGS. 3A and 3B:

Biophysical and antigenic characterization of purified trimers derived from KNH1144 SOSIP gp140. (A) Trimeric KNH1144 SOSIP gp140 was treated with 0.1% (v/v) ionic (SDS) and non-ionic (NP40, Nonidet-P40; Tw20 (Tween®-20); TX100 (Triton X-100) detergents for 1 h at 25° C. before BN-PAGE analysis. (B) Trimeric KNH1144 SOSIP gp140 was incubated with 0.5 μg/ml of the indicated MAbs or proteins, and then immunoprecipitated with protein G sepharose prior to analysis by SDS-PAGE.

FIGS. 4A-AF: Inhibition of infection by HIV-1_{JR-FL} (filled circles) or HIV-1_{KNH1144} (open circles) Env-pseudotyped viruses by MAbs and fusion inhibitors. The viruses were incubated with the indicated concentrations of the inhibitors for 1 h prior to addition to U87.CD4.CCR5 cells. The luciferase content of cell lysates was determined after 3-4 days.

FIG. 5: Analysis of purified KNH1144 SOSIP R6 gp140 trimer and gp120 monomer. Purified KNH1144 gp120 monomer (left panel, gp120) and SOSIP R6 gp140 trimer were analyzed by reducing (left panel, SOSIP R6, Red) and non-reducing SDS-PAGE (left panel, SOSIP R6, NR). Proteins were visualized by Coomassie G-250 stain. Purified trimer was also analyzed via ARP3119 western blot on non-reducing SDS-PAGE to examine presence of SDS-insoluble aggregates (middle panel, Anti-Env blot). The numbers on the left represent the migratory positions of the molecular weight standard proteins. The right panel shows BN-PAGE analysis of purified trimer, either untreated or treated with Tween® 20 (SOSIPR6, −/+lanes) and purified gp120 monomer in absence or presence of Tween® 20 treatment (gp120, −/+lanes). Arrows indicate high molecular weight (HMW) aggregate, trimer and gp120 monomer species. M stands for the 669 k thyroglobulin and 440 k ferritin molecular weight protein standards.

FIGS. 6A-6D: Tween® 20 conversion experiments. (A) Dose response: Purified KNH1144 SOSIP R6 gp140 trimer was incubated with 0 (no detergent control), or 0.1, 0.05, 0.01, 0.001, or 0.0001% Tween® 20 and analyzed by BN-PAGE and Coomassie G-250 stain. Arrows point to HMW aggregate and trimer species. M stands for the 669 k thyroglobulin and 440 k ferritin molecular weight protein standards. (B) Time course: Purified KNH1144 SOSIP R6 gp140 trimer was incubated with Tween® 20 for 5 min (left panel) or 10 min (right panel). Trimer was either untreated (−lane) or Tween® 20 treated (+lane). Arrows indicate trimer and HMW aggregate bands. (C) Temperature effect: Purified KNH1144 SOSIP R6 gp140 trimer was either untreated (−lane) or treated with Tween® 20 at on ice (0), room temperature (RD or 37° C. Reactions were analyzed by BN-PAGE and Coomassie G-250 stain. Arrows indicate HMW aggregate and trimer proteins. (D) Tween® 20 effect on HMW aggregate and dimer fractions: A preparation composed predominantly of HMW aggregate (>80%) was untreated (left panel, −lane), or incubated with Tween® 20 (left panel, +lane), and analyzed by BN-PAGE and Coomassie G-250 stain. Solid arrows indicate HMW aggregate and trimer proteins. Preparations composed of HMW aggregate, dimers and monomers were untreated (right panel, −lane) or incubated with Tween® 20 (right panel, +lane) and analyzed by BN-PAGE and Coomassie G-250 stain. Arrows on the right hand side point to aggregate, trimer, dimer and monomer species.

FIG. 7: Size Exchange Chromatography (SEC) analysis of KNH1144 SOSIP R6 gp140 trimer. KNH1144 SOSIP R6 gp140 trimer was resolved on a Superdex 200 10/300 GL column in TN-500 buffer containing 0.05% Tween® 20 (TNT-500). The A₂₈₀ protein profile of the run is shown in the middle panel. Fractions B7-C3 from the run were analyzed by BN-PAGE, followed by silver stain (bottom panel). Arrows to the side of the BN-PAGE image point to the trimer. The vertical arrow in the BN-PAGE indicates the peak signal of the trimer in fraction B12. The arrow in the middle chromatograph corresponds to fraction B12.

FIG. 8: Effect of Tween® 20 treatment on KNH1144 SOSIP R6 HMW aggregate antigenicity. Lectin ELISA of untreated and Tween® 20 treated KNH1144 SOSIP R6 HMW aggregate: Untreated or Tween® 20-treated HMW aggregate were bound to GNA lectin coated ELISA plates and probed with 2G12, b6, b12, CD4-IgG2, and HIVIg. The panels represent their respective binding curves. Antibody affinity to the untreated HMW aggregate is represented by the curve having diamond lines. Affinity to the Tween® 20 treated HMW aggregate is represented by curve having square lines. The Y-axis represents the colorimetric signal at OD492 and the X-axis represents antibody concentration in [ug/ml]. Lectin ELISA of untreated and Tween® 20-treated KNH1144 SOSIP R6 gp140 trimer: Untreated or Tween® 20 treated trimer (containing 10-15% HMW aggregate) were bound to GNA lectin coated ELISA plates and probed with 2G12, b6, b12, and CD4-IgG2. The panels represent their respective binding curves. Antibody affinity to the untreated trimer is represented by the curve having diamond lines. Affinity to the Tween® 20 treated trimer is represented by the curve having square lines. The Y-axis represents the colorimetric signal at OD492 and the X-axis represents antibody concentration in [ug/ml].

FIG. 9: Effect of Tween® 20 treatment on KNH1144 SOSIP R6 gp140 trimer binding to DEAE anion exchange column. Purified KNH1144 SOSIP R6 gp140 trimer, spiked with alpha-2 macroglobulin (a₂M) contaminant, was either untreated or treated with Tween® 20. Following treatment, sample was applied over an anion exchange column (DEAE HiTrap FF 1 ml column) (Load). Flow through (FT) fractions were collected and the column was washed (Wash). The column was eluted (Elution) and fractions were analyzed over BN-PAGE, followed by Coomassie G-250 stain. The top panel shows fractions analyzed from the untreated control trimer DEAE application. The bottom panel shows fractions analyzed from the Tween® 20 treated trimer DEAE application. Arrows point to trimer and a₂M contaminant proteins. M stands for the 669 k thyroglobulin and 440 k ferritin molecular weight protein standards. Asterisks highlight the fraction where the trimer is found.

FIG. 10: Negative stain electron micrographs of KNH1144 SOSIP R6 gp140 trimers. KNH1144 SOSIP R6 gp140 trimers were analyzed by negative stain electron microscopy. Bar=50 nm.

FIG. 11: SEC analysis of KNH1144 gp120 monomer: KNH1144 gp120 monomer was resolved on a Superdex 200 10/300 GL column in TN-500 buffer. The top chromatograph shows its A₂₈₀ protein profile of the run. As a control, JR-FL gp120 monomer was resolved in a similar manner and its A₂₈₀ protein profile is displayed in the bottom chromatograph. The observed retention times for both monomers and their apparent calculated molecular weights are indicated.

FIG. 12: Tween® 20 effect on a₂M: Purified a₂M was incubated with Tween® 20 (+lane) or was untreated (−lane). Reactions were analyzed by BN-PAGE and Coomassie stain. Arrow indicates a₂M band.

FIG. 13: Amino acid sequence (SEQ ID NO:1) of the modified HIV-1 KNH1144 gp140 isolate.

FIG. 14: Nucleic acid sequence (SEQ ID NO:13) and amino acid sequence (SEQ ID NO:10) of the HIV-1 5768.4 isolate.

FIG. 15: Detergent “collapse” effect on subtype B 5768.4 HMW aggregate. 0.24 ug of purified subtype B 5768.4 SOSIP R6 gp140 trimer was incubated with 0.05 or 0.1% Tween 20. In addition, similar incubations were performed with NP40, Triton X-100, and SDS (at a final concentration of 0.1%) or the trimer preparation was untreated. Reactions were separated on BN-PAGE and visualized by coomassie G-250 stain. Arrows indicate HMW aggregate, a₂M contaminant, trimer and monomer proteins. M stands for the 669 k thyroglobulin and 440 k ferritin molecular weight protein standards.

FIG. 16: Rabbit immunogenicity study design comparing KNH1144 SOSIP trimer as an immunogen with gp120 monomer as an immunogen.

FIG. 17: Neutralization of homologous env-pseudotyped HIV-1_{KNH1144} by SOSIP and gp120 antisera generated in the rabbit immunogenicity study (Comparison: KNH1144 SOSIP vs. gp120).

FIG. 18: Neutralization of heterologous env-pseudotyped HIV-1_{MBC8}, HIV-1_{NL4-3}, HIV-1_{MN}, and HIV-1_{SF162} by KNH1144 SOSIP and gp120 antisera generated in the rabbit immunogenicity study (Comparison: Ribi-adjuvanted KNH1144 SOSIP vs. gp120).

FIG. 19: Neutralization of heterologous env-pseudotyped HIV-1_{MBC8}, HIV-1_{NL4-3}, HIV-1_{MN}, and HIV-1_{SF162} by SOSIP antisera generated in the rabbit immunogenicity study (Comparison: Quil A vs. Ribi adjuvant used with Tween 20® treated KNH1144 SOSIP gp140).

FIG. 20: Neutralization of heterologous env-pseudotyped HIV-1_{MBC8}, HIV-1_{NL4-3}, HIV-1_{MN}, and HIV-1_{SF162} by SOSIP antisera generated in the rabbit immunogenicity study (Comparison: presence and absence of Tween 20®).

FIGS. 21A, 21B and 21C: ELISA analysis of total anti-gp120 immune response induced by immunization of animals with KNH1144 SOSIP Env trimer or KNH1144 Env gp120 monomer as immunogens. (FIG. 21A): Group I rabbits immunized with monomeric KNH1144 gp120 in the presence of Tween; Group II rabbits immunized with KNH1144 SOSIP in the presence of Tween 20®, 30 ug of protein per injection using Quil A as adjuvant. (FIG. 21B): Group III rabbits immunized with monomeric KNH1144 gp120 in the absence of Tween; Group IV rabbits immunized with KNH1144 SOSIP in the absence of Tween 20®, 30 ug of protein per injection using Quil A as adjuvant. (FIG. 21C): Group V rabbits immunized with KNH1144 monomeric gp120 in the presence of Tween 20® and Group VI using KNH1144 SOSIP in the presence of Tween, 100 ug of protein for the first immunization, 30 ug of protein for subsequent immunizations, using RIBI as adjuvant.

### DETAILED DESCRIPTION OF THE INVENTION

### Definitions

As used in this application, except as otherwise expressly provided herein, each of the following terms shall have the meaning set forth below.

The following standard abbreviations are used throughout the specification to indicate specific amino acids: A=ala=alanine; R=arg=arginine; N=asn=asparagine; D=asp=aspartic acid; C=cys=cysteine; Q=gln=glutamine; E=glu=glutamic acid; G=gly=glycine; H=his=histidine; I=ile=isoleucine; L=leu=leucine; K=lys=lysine; M=met=methionine; F=phe=phenylalanine; P=pro=proline; S=ser=serine; T=thr=threonine; W=trp=tryptophan; Y=tyr=tyrosine; V=val=valine; B=asx=asparagine or aspartic acid; Z=glx=glutamine or glutamic acid.

An “A511C mutation” refers to a point mutation of amino acid 511 in the HIV-1 KNH1144 isolate gp120 from alanine to cysteine. Because of sequence and sequence numbering variability among different HIV strains and isolates, it will be appreciated that this amino acid may not be at position 511 in all other HIV isolates. For example, in HIV-1_{JR-FL} the corresponding amino acid is A492 (Genbank Accession No. U63632); in HIV-1_{HXB2} the corresponding amino acid is A501 (Genbank Accession No. AAB50262); and in HIV-1_{NL4-3} it is A499 (Genbank Accession No. AAA44992). The amino acid may also be an amino acid other than alanine or cysteine which has similar polarity or charge characteristics, for example. This invention encompasses the replacement of such amino acids by cysteine, as may be readily identified in other HIV isolates by those skilled in the art.

“I571P” refers to a point mutation wherein the isoleucine residue at position 571 of a polypeptide chain is replaced by a proline residue.

A “T617C mutation” refers to a point mutation of amino acid 617 in HIV-1 KNH1144 isolate gp41 ectodomain from threonine to cysteine. Because of sequence and sequence numbering variability among different HIV strains and isolates, it will be appreciated that this amino acid will not be at position 617 in all other HIV isolates. For example, in HIV-1_{JR-FL} the corresponding amino acid is T596 (Genbank Accession No. U63632); in HIV-1_{HXB2} the corresponding amino acid is T605 (Genbank Accession No. AAB50262); and in HIV-1_{NL4-3} the corresponding amino acid is T603 (Genbank Accession No. AAA44992). The amino acid may also be an amino acid other than threonine or cysteine which has similar polarity or charge characteristics, for example. This invention encompasses cysteine mutations in such amino acids, which can be readily identified in other HIV isolates by those skilled in the art. This invention encompasses the replacement of such amino acids by cysteine, as may be readily identified in other HIV isolates by those skilled in the art

An “A519C mutation” refers to a point mutation of amino acid 519 in HIV-1 5768.4 isolate gp120 from alanine to cysteine. Because of sequence and sequence numbering variability among different HIV strains and isolates, it will be appreciated that this amino acid will not be at position 519 in all other HIV isolates. For example, in HIV-1_{JR-FL} the corresponding amino acid is A492 (Genbank Accession No. 1.163632), in HIV-1_{HXB2} the corresponding amino acid is A501 (Genbank Accession No. AAB50262) and in HIV-1_{NL4-3} it is A499 (Genbank Accession No. AAA44992). The amino acid may also be an amino acid other than alanine which has similar polarity or charge characteristics, for example. This invention encompasses cysteine mutations in such amino acids, which can be readily identified in other HIV isolates by those skilled in the art. This invention encompasses the replacement of such amino acids by cysteine, as may be readily identified in other HIV isolates by those skilled in the art.

“I579P” refers to a point mutation wherein the isoleucine residue at position 579 of a polypeptide chain is replaced by a proline residue.

A “T625C mutation” refers to a point mutation of amino acid 625 in HIV-1 5768.4 isolate gp41 ectodomain from threonine to cysteine. Because of sequence and sequence numbering variability among different HIV strains and isolates, it will be appreciated that this amino acid will not be at position 625 in all other HIV isolates. For example, in HIV-1_{JR-FL} the corresponding amino acid is T596 (Genbank Accession No. U63632), in HIV-1_{HXB2} the corresponding amino acid is T605 (Genbank Accession No. AAB50262) and in HIV-1_{NL4-3} the corresponding amino acid is T603 (Genbank Accession No. AAA44992). The amino acid may also be an amino acid other than threonine which has similar polarity or charge characteristics, for example. This invention encompasses the replacement of such amino acids by cysteine, as may be readily identified in other HIV isolates by those skilled in the art.

“HIV” refers to the human immunodeficiency virus. HIV includes, without limitation, HIV-1. HIV may be either of the two known types of HIV, i.e., HIV-1 or HIV-2. The HIV-1 virus may represent any of the known major subtypes or clades (e.g., Classes A, B, C, D, E, F, G and H) or outlying subtype (Group O).

“gp140 envelope” refers to a protein having two disulfide-linked polypeptide chains, the first chain comprising the amino acid sequence of the HIV gp120 glycoprotein and the second chain comprising the amino acid sequence of the water-soluble portion of HIV gp41 glycoprotein (“gp41 portion”). HIV gp140 protein includes, without limitation, HIV protein, i.e., envelope (Env) protein, wherein the gp41 portion comprises a point mutation such as I571P. A gp140 envelope comprising such mutation is encompassed by the terms “HIV SOS gp140”, as well as “HIV gp140 monomer” or “SOSIP gp140”.

“gp41” includes, without limitation, (a) the entire gp41 polypeptide including the transmembrane and cytoplasmic domains; (b) gp41 ectodomain (gp41_{ECTO}); (c) gp41 modified by deletion or insertion of one or more glycosylation sites; (d) gp41 modified so as to eliminate or mask the well-known immunodominant epitope; (e) a gp41 fusion protein; and (f) gp41 labeled with an affinity ligand or other detectable marker. As used herein, “ectodomain” means the extracellular region of a transmembrane protein exclusive of the transmembrane spanning and cytoplasmic regions.

“Host cells” include, but are not limited to, prokaryotic cells, e.g., bacterial cells (including gram-positive cells), yeast cells, fungal cells, insect cells and animal cells. Suitable animal cells include, but are not limited to HeLa cells, COS cells, CV1 cells and various primary mammalian cells. Numerous mammalian cells can be used as hosts, including, but not limited to, mouse embryonic fibroblast NIH-3T3 cells, CHO cells, HeLa cells, L(tk-) cells and COS cells. Mammalian cells can be transfected by methods well known in the art, such as calcium phosphate precipitation, electroporation and microinjection. Electroporation can also be performed in vivo as described previously (see, e.g., U.S. Pat. Nos. 6,110,161; 6,262,281; and 6,610,044).

“Immunizing” means generating an immune response to an antigen in a subject. This can be accomplished, for example, by administering a primary dose of an antigen, e.g., a vaccine, to a subject, followed after a suitable period of time by one or more subsequent administrations of the antigen or vaccine, so as to generate in the subject an immune response against the antigen or vaccine. A suitable period of time between administrations of the antigen or vaccine may readily be determined by one skilled in the art, and is usually on the order of several weeks to months. Adjuvant may or may not be co-administered.

“Nucleic acid” refers to any nucleic acid or polynucleotide, including, without limitation, DNA, RNA and hybrids thereof. The nucleic acid bases that form nucleic acid molecules can be the bases A, C, T, G and U, as well as derivatives thereof. Derivatives of these bases are well known in the art and are exemplified in PCR Systems, Reagents and Consumables (Perkin-Elmer Catalogue 1996-1997, Roche Molecular Systems, Inc., Branchburg, N.J., USA).

A “vector” refers to any nucleic acid vector known in the art. Such vectors include, but are not limited to, plasmid vectors, cosmid vectors and bacteriophage vectors. For example, one class of vectors utilizes DNA elements which are derived from animal viruses such as animal papilloma virus, polyoma virus, adenovirus, vaccinia virus, baculovirus, retroviruses (RSV, MMTC or MoMLV), Semliki Forest virus or SV40 virus. The eukaryotic expression plasmid PPI4 and its derivatives are widely used in constructs described herein. However, the invention is not limited to derivatives of the PPI4 plasmid and may include other plasmids known to those skilled in the art.

In accordance with the invention, numerous vector systems for expression of recombinant proteins may be employed. For example, one class of vectors utilizes DNA elements which are derived from animal viruses such as bovine papilloma virus, polyoma virus, adenovirus, vaccinia virus, baculovirus, retroviruses (RSV, MMTV or MoMLV), Semliki Forest virus or SV40 virus. Additionally, cells which have stably integrated the DNA into their chromosomes may be selected by introducing one or more markers which allow for the selection of transfected host cells. The marker may provide, for example, prototropy to an auxotrophic host, biocide (e.g., antibiotic) resistance, or resistance to heavy metals such as copper or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals. The cDNA expression vectors incorporating such elements include those described by (Okayama and Berg, 1983).

“Pharmaceutically acceptable carriers” are well known to those skilled in the art and include, but are not limited to, 0.01-0.1M and preferably 0.05M phosphate buffer, phosphate-buffered saline (PBS), or 0.9% saline. Additionally, such pharmaceutically acceptable carriers may include, but are not limited to, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers, diluents and excipients include water, alcoholic/aqueous solutions, emulsions or suspensions, saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Preservatives and other additives, such as, for example, antimicrobials, antioxidants, chelating agents, inert gases, and the like may also be included with all the above carriers.

Adjuvants are formulations and/or additives that are routinely combined with antigens to boost immune responses. Suitable adjuvants for nucleic acid based vaccines include, but are not limited to, saponins, Quil A, imiquimod, resiquimod, interleukin-12 delivered in purified protein or nucleic acid form, short bacterial immunostimulatory nucleotide sequences such as CpG-containing motifs, interleukin-2/Ig fusion proteins delivered in purified protein or nucleic acid form, oil in water micro-emulsions such as MF59, polymeric microparticles, cationic liposomes, monophosphoryl lipid A, immunomodulators such as Ubenimex, and genetically detoxified toxins such as E. coli heat labile toxin and cholera toxin from Vibrio. Such adjuvants and methods of combining adjuvants with antigens are well known to those skilled in the art.

Adjuvants suitable for use with protein immunization include, but are not limited to, alum; Freund's incomplete adjuvant (FIA); saponin; Quil A; QS-21; Ribi, Ribi Detox; monophosphoryl lipid A (MPL) adjuvants such as Enhanzyn™; nonionic block copolymers such as L-121 (Pluronic; Syntex SAF); TiterMax Classic adjuvant (block copolymer, CRL89-41, squalene and microparticulate stabilizer; Sigma-Aldrich); TiterMax Gold Adjuvant (new block copolymer, CRL-8300, squalene and a sorbitan monooleate; Sigma-Aldrich); Ribi adjuvant system using one or more of the following: monophosphoryl lipid A, synthetic trehalose, dicorynomycolate, mycobacterial cell wall skeleton incorporated into squalene and polysorbate-80; Corixa); RC-552 (a small molecule synthetic adjuvant; Corixa); Montanide adjuvants (including Montanide IMS111X, Montanide IMS131x, Montanide IMS221x, Montanide IMS301x, Montanide ISA 26A, Montanide ISA206, Montanide ISA 207, Montanide ISA25, Montanide ISA27, Montanide ISA28, Montanide ISA35, Montanide ISA50V, Montanide ISA563, Montanide ISA70, Montanide ISA 708, Montanide ISA740, Montanide ISA763A, and Montanide ISA773; Seppic Inc., Fairfield, N.J.); and N-Acetylmuramyl-L-alanyl-D-isoglutamine hydrate (Sigma-Aldrich). Methods of combining adjuvants with antigens are well known to those skilled in the art.

Because current vaccines depend on generating antibody responses to injected antigens, commercially available adjuvants have been developed largely to enhance these antibody responses. To date, the only FDA-approved adjuvant for use with human vaccines is alum. However, although alum helps boost antibody responses to vaccine antigens, it does not enhance T cell immune responses. Thus, adjuvants that are able to boost T cell immune responses after a vaccine is administered are also contemplated for use.

It is also known to those skilled in the art that cytotoxic T lymphocyte and other cellular immune responses are elicited when protein-based immunogens are formulated and administered with appropriate adjuvants, such as ISCOMs and micron-sized polymeric or metal oxide particles. Certain microbial products also act as adjuvants by activating macrophages, lymphocytes and other cells within the immune system, and thereby stimulating a cascade of cytokines that regulate immune responses. One such adjuvant is monophosphoryl lipid A (MPL) which is a derivative of the gram-negative bacterial lipid A molecule, one of the most potent immunostimulants known. The Enhanzyn™ adjuvant (Corixa Corporation, Hamilton, Mont.) consists of MPL, mycobacterial cell wall skeleton and squalene.

Adjuvants may be in particulate form. The antigen may be incorporated into biodegradable particles composed of poly-lactide-co-glycolide (PLG) or similar polymeric material. Such biodegradable particles are known to provide sustained release of the immunogen and thereby stimulate long-lasting immune responses to the immunogen. Other particulate adjuvants include, but are not limited to, micellular particles comprising Quillaia saponins, cholesterol and phospholipids known as immunostimulating complexes (ISCOMs; CSL Limited, Victoria AU), and superparamagnetic particles. Superparamagnetic microbeads include, but are not limited to, μMACS™ Protein G and μMACS™ Protein A microbeads (Miltenyi Biotec), Dynabeads® Protein G and Dynabeads® Protein A (Dynal Biotech). In addition to their adjuvant effect, superparamagnetic particles such as μMACS™ Protein G and Dynabeads® Protein G have the important advantage of enabling immunopurification of proteins.

A “prophylactically effective amount” is any amount of an agent which, when administered to a subject prone to suffer from a disease or disorder, inhibits or prevents the onset of the disorder. The prophylactically effective amount will vary with the subject being treated, the condition to be treated, the agent delivered and the route of delivery. A person of ordinary skill in the art can perform routine titration experiments to determine such an amount. Depending upon the agent delivered, the prophylactically effective amount of agent can be delivered continuously, such as by continuous pump, or at periodic intervals (for example, on one or more separate occasions). Desired time intervals of multiple amounts of a particular agent can be determined without undue experimentation by one skilled in the art.

“Inhibiting” the onset of a disorder means either lessening the likelihood of the disorder's onset, preventing the onset of the disorder entirely, or in some cases, reducing the severity of the disease or disorder after onset. In the preferred embodiment, inhibiting the onset of a disorder means preventing its onset entirely.

“Reducing the likelihood of a subject's becoming infected with HIV-1” means reducing the likelihood of the subject's becoming infected with HIV-1 by at least two-fold. For example, if a subject has a 1% chance of becoming infected with HIV-1, a two-fold reduction in the likelihood of the subject becoming infected with HIV-1 would result in the subject having a 0.5% chance of becoming infected with HIV-1. In the preferred embodiment of this invention, reducing the likelihood of the subject's becoming infected with HIV-1 means reducing the likelihood of the subject's becoming infected with the virus by at least ten-fold.

“Subject” means any animal or artificially modified animal. Animals include, but are not limited to, humans, non-human primates, cows, horses, sheep, goats, pigs, dogs, cats, rabbits, ferrets, rodents such as mice, rats and guinea pigs, and birds and fowl, such as chickens and turkeys. Artificially modified animals include, but are not limited to, transgenic animals or SCID mice with human immune systems. In the preferred embodiment, the subject is a human.

“Exposed” to HIV-1 means contact or association with HIV-1 such that infection could result. A “therapeutically effective amount” is any amount of an agent which, when administered to a subject afflicted with a disorder against which the agent is effective, causes the subject to be treated. “Treating” a subject afflicted with a disorder shall mean causing the subject to experience a reduction, diminution, remission, suppression, or regression of the disorder and/or its symptoms. In one embodiment, recurrence of the disorder and/or its symptoms is prevented. Most preferably, the subject is cured of the disorder and/or its symptoms.

“HIV-1 infected” means the introduction of viral components, virus particles, or viral genetic information into a cell, such as by fusion of cell membrane with HIV-1. The cell may be a cell of a subject. In the preferred embodiment, the cell is a cell in a human subject.

### Embodiments of the Invention

This invention provides a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:2 and SEQ ID NO:3, respectively, said modified gp120 envelope polypeptide portion comprising a cysteine at amino acid position 511 and said modified gp41 ectodomain polypeptide portion comprising a cysteine at amino acid position 617 and a proline at amino acid position 571, wherein (i) the amino acid positions are numbered by reference to SEQ ID NO:1, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 511 and the cysteine at amino acid position 617. In one embodiment, the cysteine at position 511 is the result of an A511C mutation. In another embodiment, the cysteine at position 617 is the result of a T617C mutation. In yet another embodiment, the proline at position 571 is the result of an I571P mutation.

In one embodiment, the modified gp120 polypeptide portion comprises the consecutive amino acid sequence as set forth in SEQ ID NO:2. In another embodiment, the modified gp41 ectodomain polypeptide portion comprises the consecutive amino acid sequence as set forth in SEQ ID NO:3. In yet another embodiment, the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii).

This invention also provides a trimeric complex comprising three monomers, each of which is a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ. ID NO:2 and SEQ ID NO:3; respectively, said modified gp120 envelope polypeptide portion comprising a cysteine at amino acid position 511 and said modified gp41 ectodomain polypeptide portion comprising a cysteine at amino acid position 617 and a proline at amino acid position 571, wherein (i) the amino acid positions are numbered by reference to SEQ ID NO:1, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 511 and the cysteine at amino acid position 617. In one embodiment, the cysteine at position 511 is the result of an A511C mutation. In another embodiment, the cysteine at position 617 is the result of a T617C mutation. In yet another embodiment, the proline at position 571 is the result of an I571P mutation. In an embodiment, the trimeric complex binds a structure recognized by HIV-1, e.g., CD4, soluble CD4 (sCD4), CCR5, etc.

In one embodiment, the modified gp120 polypeptide portion comprises the consecutive amino acid sequence as set forth in SEQ ID NO:2. In another embodiment, the modified gp41 ectodomain polypeptide portion comprises the consecutive amino acid sequence as set forth in SEQ ID NO:3. In yet another embodiment, the modified gp120 polypeptide portion is further characterized by (i) the absence of one or more canonical glycosylation sites present in wild-type gp120, (ii) the presence of one or more canonical glycosylation sites absent in wild-type HIV-1 gp120, or (iii) both (i) and (ii).

This invention further provides a nucleic acid encoding a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:2 and SEQ 1D NO:3, respectively, said modified gp120 envelope polypeptide portion comprising a cysteine at amino acid position 511 and said modified gp41 ectodomain polypeptide portion comprising a cysteine at amino acid position 617 and a proline at amino acid position 571, wherein (i) the amino acid positions are numbered by reference to SEQ ID NO:1, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 511 and the cysteine at amino acid position 617. In one embodiment, the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii). In other embodiments, the nucleic acid may be DNA, cDNA, or RNA.

This invention also provides a vector comprising a nucleic acid encoding a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:2 and SEQ ID NO:3, respectively, said modified gp120 envelope polypeptide portion comprising a cysteine at amino acid position 511 and said modified gp41 ectodomain polypeptide portion comprising a cysteine at amino acid position 617 and a proline at amino acid position 571, wherein (i) the amino acid positions are numbered by reference to SEQ ID NO:1, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 511 and the cysteine at amino acid position 617. In one embodiment, the modified gp120 polypeptide portion is further characterized by (i) the absence of one or more canonical glycosylation sites present in, wild-type HIV-1 gp120, (ii) the presence of one or more canonical glycosylation sites absent in wild-type HIV-1 gp120, or (iii) both (i) and (ii). In other embodiments, the nucleic acid may be DNA, cDNA, or RNA.

This invention provides a protein comprising (a) a first polypeptide which comprises consecutive amino acids, the sequence of which corresponds to the sequence of a modified gp120 envelope polypeptide portion of a gp140 envelope of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625. In one embodiment, the cysteine at position 519 is the result of an A519C mutation. In another embodiment, the cysteine at position 625 is the result of a T625C mutation. In yet another embodiment, the proline at position 579 is the result of an I579P mutation.

In one embodiment, the modified gp120 polypeptide portion comprises the consecutive amino acid sequence as set forth in SEQ ID NO:11. In another embodiment, the modified gp41 ectodomain polypeptide portion comprises the consecutive amino acid sequence as set forth in SEQ ID NO:12. In yet another embodiment, the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii).

This invention also provides a trimeric complex comprising three monomers, each of which is a protein comprising (a) a first polypeptide which comprises consecutive amino acids, the sequence of which corresponds to the sequence of a modified gp120 envelope polypeptide portion of a gp140 envelope of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625. In one embodiment, the cysteine at position 519 is the result of an A519C mutation. In another embodiment, the cysteine at position 625 is the result of a T625C mutation. In yet another embodiment, the proline at position 579 is the result of an I579P mutation.

In one embodiment, the modified gp120 polypeptide portion comprises the consecutive amino acid sequence as set forth in SEQ ID NO:11. In another embodiment, the modified gp41 ectodomain polypeptide portion comprises the consecutive amino acid sequence as set forth in SEQ ID NO:12. In yet another embodiment, the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii).

This invention further provides a nucleic acid encoding a protein comprising (a) a first polypeptide which comprises consecutive amino acids, the sequence of which corresponds to the sequence of a modified gp120 envelope polypeptide portion of a gp140 envelope of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625. In one embodiment, the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii). In other embodiments, the nucleic acid may be DNA, cDNA, or RNA.

This invention also provides a vector comprising a nucleic acid encoding a protein comprising (a) a first polypeptide which comprises consecutive amino acids, the sequence of which corresponds to the sequence of a modified gp120 envelope polypeptide portion of a gp140 envelope of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625. In one embodiment, the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii). In other embodiments, the nucleic acid may be DNA, cDNA, or RNA.

This invention further provides a host cell comprising a vector as above-described. In one embodiment, the host cell is a eukaryotic cell. In another embodiment, the host cell is a prokaryotic cell. The prokaryotic cell may be a bacterial cell.

This invention also provides a composition comprising a trimeric complex of the invention, and a pharmaceutically acceptable carrier, excipient or diluent. In one embodiment, the composition further comprises an adjuvant. In another embodiment, the composition further comprises a non-ionic detergent. In other embodiments, the trimeric complex is comprised of three monomers, each of which is a protein comprising (a) a first polypeptide which comprises consecutive amino acids, the sequence of which corresponds to the sequence of a modified gp120 envelope polypeptide portion of a gp140 envelope of an HIV-1 KNH1144 isolate, or of an HIV-1 5768.4 isolate, or a quasi-species thereof; and (b) a second polypeptide which comprises consecutive amino acids, the sequence of which corresponds to the sequence of a modified gp41 ectodomain polypeptide portion of the gp140 envelope of the HIV-1 KNH1144 isolate or the HIV-1 5768.4 isolate or such quasi-species thereof, wherein the trimer complexes have the properties as described hereinabove.

This invention provides a method for eliciting an immune response against HIV-1 or an HIV-1 infected cell in a subject comprising administering to the subject an amount of a trimeric complex of the invention effective to elicit the immune response in the subject. In one embodiment, the trimeric complex is administered in a single dose. In another embodiment, the trimeric complex is administered in multiple doses. In yet another embodiment of the invention, the trimeric complex is administered as part of a prime-boost regimen.

This invention also provides a method for preventing a subject from becoming infected with HIV-1 comprising administering to the subject a prophylactically effective amount of a trimeric complex of the invention so as to thereby prevent the subject from becoming infected with HIV-1.

This invention further provides a method for reducing the likelihood of a subject becoming infected with HIV-1 comprising administering to the subject an amount of a trimeric complex of the invention effective to reduce the likelihood of the subject becoming infected with HIV-1. In one embodiment, the subject has been exposed to HIV-1.

This invention also provides a method for delaying the onset of, or slowing the rate of progression of, an HIV-1-related disease in an HIV-1-infected subject which comprises administering to the subject an amount of a trimeric complex of the invention effective to delay the onset of, or slowing the rate of progression of, the HIV-1-related disease in the subject.

In one embodiment of the invention, a quasi-species of the HIV-1 KNH1144 isolate comprises an HIV-1 viral isolate having a gp140 envelope sequence comprising less than or equal to 1% variation in sequence identity relative to SEQ ID NO:1. For example, the quasi-species comprises the sequence set forth in GenBank Accession No. AF457066.

In an embodiment of the invention, the mutated furin recognition sequence comprises amino acids 518 to 523 of SEQ ID NO:1.

In another embodiment of the invention, the quasi-species of the HIV-1 5768.4 isolate comprises an HIV-1 viral isolate having a gp140 envelope sequence comprising less than or equal to 1% variation in sequence identity relative to SEQ ID NO:10. In another embodiment, the quasi-species comprises the sequence set for in GenBank Accession No. AY835435.

In an embodiment of the invention, the mutated furin recognition sequence comprises amino acids 526 to 531 of SEQ ID NO:10.

The invention also provides an isolated nucleic acid having the sequence as set forth in SEQ ID NO:13, which encodes a modified gp120 polypeptide portion and a modified gp41 ectodomain polypeptide portion of the gp140 envelope protein of an HIV-1 5768.4 isolate.

Finally, the invention provides a trimeric complex of the invention, further comprising a non-ionic detergent. In one embodiment, the non-ionic detergent is a polyethylene type detergent. The polyethylene type detergent may be poly(oxyethylene)sorbitan monolaureate or poly(oxyethylene)sorbitan monooleate. The poly(oxyethylene)sorbitan monolaureate may be poly(oxyethylene)(20)sorbitan monolaureate. The trimers in non-ionic detergent according to this invention are stable for days, weeks and months, e.g., greater than one week, greater than two weeks, greater than one month, greater than two months, or greater than six months to years, for example, at ˜4° C.-25° C., at room temperature (e.g., ˜16° C.-25° C.), or frozen.

The trimeric complexes (trimers) of this invention may be used as antigens, immunogens, or as vaccines against HIV infection. The trimers may be used alone or in combination with other antigens and/or vaccines, with or without adjuvants. The trimers of the invention therefore may be used as immunogens to promote the production of neutralizing antibodies against HIV. The trimeric complexes of this invention may also be used to generate monoclonal antibodies which may be used, for example, in detection assays.

This invention is illustrated in the Experimental Details section which follows. This section is set forth to aid in an understanding of the invention but is not intended to, and should not be construed to limit in any way the invention as set forth in the claims which follow thereafter.

Experimental Details I

Introduction

The generation of an antibody response capable of neutralizing a broad range of clinical isolates remains an important goal of human immunodeficiency virus type 1 (HIV-1) vaccine development. Vaccines involving the use of envelope glycoprotein, (Env)-based vaccine candidates, need to encompass the extensive genetic diversity of circulating HIV-1 strains. Described herein is the generation of soluble, stabilized, proteolytically cleaved, trimeric forms of Env (SOSIP gp140 proteins) based on contemporary Env subtype A viruses from East Africa. The construction, purification and characterization of such complex Env proteins are described. The successful production and functional evaluation of stabilized trimers from one such protein, KNH1144 SOSIP gp140, are particularly exemplified in accordance with the present invention.

Materials and Methods

Monoclonal Antibodies and Sera:

The CD4-immunoglobulin G2 (CD4-IgG2) protein has been previously described.²⁹ The human monoclonal IgG b12,²⁸ b6,⁴⁸ 2F5,³¹ 4E10⁴⁹ and 2G12³⁰ were obtained from Dr. Dennis Burton (The Scripps Research Institute, La Jolla, Calif.) or Dr. Herman Katinger (University of Natural Resources and Applied Life Sciences, Austria, Vienna). Human monoclonal antibodies (MAbs) 17b directed against a complex gp120 epitope that becomes preferentially exposed after CD4 binding^{50,51} and 15e directed against an epitope that overlaps with the CD4 binding site on gp120⁵², were obtained from Dr. James Robinson (Tulane University Medical School, New Orleans, La.). PA1 is a V3_{JR-FL}-specific murine MAb, as defined by its ability to bind a cyclic V3_{JR-FL} peptide, but not a cyclic V3_{HXB2} peptide or V3 loop-deleted gp120_{JR-FL}, in an ELISA (Progenics Pharmaceuticals Inc., Tarrytown, N.Y.). MAb CA13 (ARP 3119; AIDS Reagent Programme, NIBSC, Potters Bar, UK; contributed by Ms C. Arnold) is a gp120-binding antibody elicited in mice by priming with vaccinia-expressed Env 92/UG/029 and boosting with soluble recombinant 92/UG/029 Env. CA13 cross-reacts with both native and denatured gp120 from Env subtypes A to F, suggesting that its epitope is continuous and fairly well conserved. D7324 is a sheep antibody directed against the gp120 C-terminus (#6205; Cliniqa Corp., Fallbrook, Calif.). HIVIg is an Ig preparation purified from a pool of sera from HIV-1 infected individuals, and was obtained from the NIH AIDS Research and Reference Reagent Program, Germantown, Md. LTNP-2 (derived from patient AD) and FDA#2 are polyclonal antisera derived from HIV-1 infected individuals.^{53,54} The anti-CCR5 murine MAb, PA14 (Progenies Inc.),⁵⁵ and the CCR5-specific small molecule inhibitors SCH-C⁵⁶ and SCH-D⁵⁷ (synthesized by Cardinal Health Inc., Dublin, Ohio) have been described. The gp41 peptide inhibitor, T-20, was synthesized by the American Peptide Company Inc., CA. AZT was purchased from Sigma-Aldrich, St. Louis, Mo. Rabbit immune sera raised against monomeric gp120s derived from HIV-1_{BB359} (Env subtype A), HIV-1_{Q23-17} (A), HIV-1_{JR-FL} (B), HIV-1_{BaL} (B), HIV-1_{DU422} (C) and _{DU179} (C) strains, were obtained from Drs. Robert Doms and Bridget Puffer (University of Pennsylvania, Philadelphia, Pa.).

Env Expression Constructs:

Functional HIV-1 env subtype A clones KER2008 (accession number, AF457052), KNH1144 (AF457066), KNH1207 (AF457068) and KNH1211 (AF457070) were supplied by Dr. Francine McCutchan (US Military HIV Research Program, Henry M. Jackson Foundation, Rockville, Md.). Each clone is a homogeneous clade A sequence derived by PCR from peripheral blood mononuclear cells (PBMC) collected during 1999-2000.⁵ HIV-1 env clones Q23-17 (accession number, AF004885) and BB359 were made by J.O.^{46,47} They are homogenously clade A sequences derived by PCR from PBMC (BB359) or from the proviral DNA of a cultured virus stock (Q23-17). Both clones were derived from isolates made during the early stages of infection.

Soluble gp140 proteins were all expressed from the high level mammalian expression vector, pPPI4, as described elsewhere.³ Briefly, a soluble wild-type (Wt) gp140 was amplified from the subtype A env gene template by PCR using sequence-specific primers based on those described²³ and were cloned into pPPI4 using the restriction enzymes KpnI and BstBI. The Env motif (VEKL) upstream from the KpnI junction and the end of the tissue plasminogen activator leader was altered by mutagenesis (Stratagene, La Jolla, Calif.) to be homologous with the Env subtype A template. The modifications necessary to make the soluble SOS, SOSIP and SOSIP.R6 gp140 proteins have been described.^{23,43,44} A monomeric gp120-expressing construct for each subtype A Env was made by introduction of two stop codons into the wild type (Wt) gp140 sequence immediately following the primary cleavage site, using site-directed mutagenesis. Purified, monomeric HIV-1 subtype B gp120_{JR-FL} and the HIV-1 subtype C Env gp120_{DU151} were expressed from Chinese hamster ovary (CHO) cells (Progenics)⁵⁸. gp120_{BaL} was expressed from a codon-optimized BaL env gene, supplied by Dr. Timothy Fouts (Institute of Human Virology, Baltimore, Md.)⁵⁹.

A membrane-bound Wt gp140Δct fragment, containing the gp41 transmembrane domain but with a truncated cytoplasmic tail, was inserted into the pPPI4 mammalian vector essentially as described⁶⁰, except that the cytoplasmic tail was truncated at amino acid 711 by introducing a stop codon at position 712⁶¹. HIV-1_{JR-FL} Wt gp160 Env, expressed from the pSVIII vector, was donated by Dr. Dennis Burton.⁶² The sequence integrity of all clones was confirmed prior to use. The numbering of individual amino acid residues in all of the clones is based on the numbering of residues in the HXBc2 Env, according to convention.

Protein Expression by Transient Transfection:

The human embryonic kidney cell line, HEK 293T, was used for the transient expression of all proteins, as previously described.^{23,60} Five hours post-transfection, 293T cells were washed with Dulbecco's Modified Eagle's Medium supplemented with 0.05% bovine serum albumin and antibiotics. Forty-eight hours after transfection, supernatants were collected, and a cocktail of protease inhibitors (Roche Diagnostics, Indianapolis, Ind.) was added to minimize protein degradation. The supernatants were clarified by filtration through a 0.45 μm filter and concentrated approximately 10-fold using the Amicon ultra-centrifugal filter system (Millipore, Billerica, Mass.). Aliquots of the concentrated material were stored at −80° C. Assessments of Env cleavage and oligomer formation were made using Tris-glycine sodium dodecyl sulfate polyacrylamide gel electrophoresis (SDS-PAGE), with or without addition of the reducing agent dithiothreitol (DTT; 100 mM), or by Blue-Native (BN)-PAGE, as previously described^{40,43,45}.

Purification of Oligomeric Env Protein:

Trimeric SOSIP gp140 was purified from transient transfection supernatants as previously described.⁴⁵ Briefly, supernatants were concentrated and then fractionated by size-exclusion chromatography (SEC) using an analytical Superdex 200 HR10/30 column (Amersham-Pharmacia, Piscataway, N.J.) equilibrated with phosphate-buffered saline (PBS), pH 7.0. The column was calibrated with protein standards of known molecular weights (HMW Gel filtration Calibration Kit; Amersham-Pharmacia). SDS-PAGE and BN-PAGE were used to characterize the Env protein forms in the various fractions eluted (2000) from the size-exclusion column^{40,45}

Immunoprecipitation and Biophysical Stability of Oligomeric Env Species:

The antigenic structures of the monomeric gp120 and trimeric SOSIP gp140 proteins were analyzed in immunoprecipitation assays using the Seize Protein A/G Coated Plate IP kit, according to the manufacturer's instructions (Pierce Biotechnology, Rockford, Ill.).

The oligomeric stability of purified SOSIP gp140 trimers was determined essentially as previously described.⁴⁴ Briefly, 50 ng of protein was treated with 0.1% (v/v) SDS, Nonidet P-40, Tween®-20 or Triton X-100 for 1 h at room temperature, followed by analysis on BN-PAGE. The various oligomeric forms of Env resolved on the gel were detected by CA13 immunoblotting.

Antigenicity of Monomeric gp120 Derived from Subtype A Envs:

The binding of MAbs, CD4-IgG2 and rabbit immune sera to monomeric gp120 was measured by ELISA using the appropriate anti-species alkaline phosphatase conjugate and the AMPAK colorimetric detection system (Dako Cytomation, Carpinteria, Calif.), as previously described.^{63,64}.

Env-Pseudotyped HIV-1 Neutralization Assays:

The generation and quantification of Env-pseudotyped HIV-1 viruses and their subsequent use in neutralization assays have been described previously.^{45,65}. U87.CD4.CCR5 target cells were used because the input pseudoviruses had the R5 phenotype. The amount of input pseudovirus was normalized by infectivity (virus titer). Virus infection was measured by determining luciferase expression in relative light units (RLU) in target cell lysates, according to the manufacturer's instructions (Promega, Madison, Wis.).

Results

Assessment of Env Cleavage and Oligomer Formation of Modified Soluble gp140 Proteins

The design and construction of soluble SOS, SOSIP and SOSIP.R6 gp140 proteins based on the HIV-1_{JR-FL} Env sequence, and their expression from the high efficiency mammalian vector, pPPI4, have been described previously^{23,43-45}. A similar panel of soluble gp140-expressing constructs was generated from six homogeneously subtype A Env templates derived from the following HIV-1 strains^{5,46,47}: KER2008, KNH1144, KNH1207, KNH1211, BB359 and Q23-17.

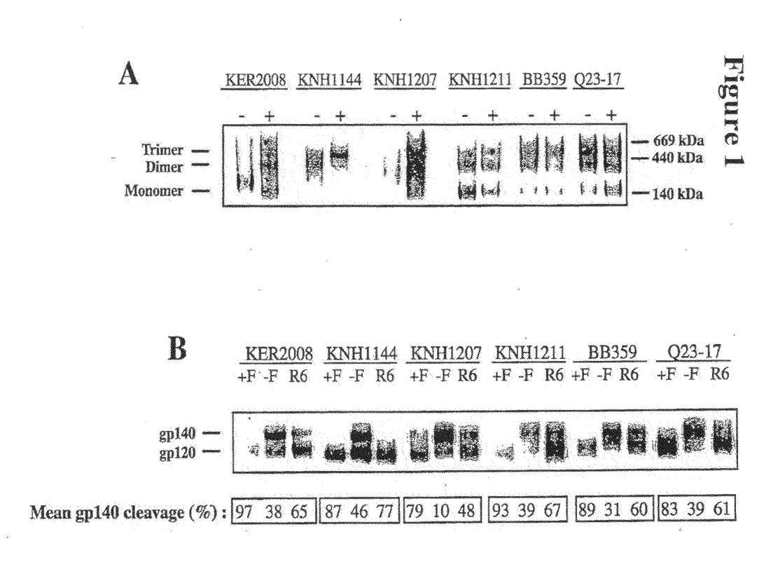

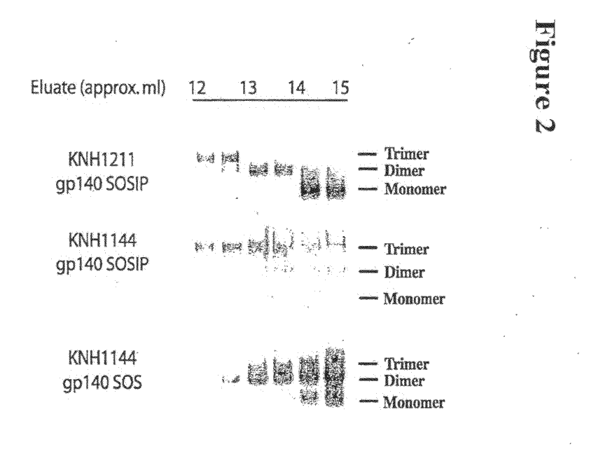

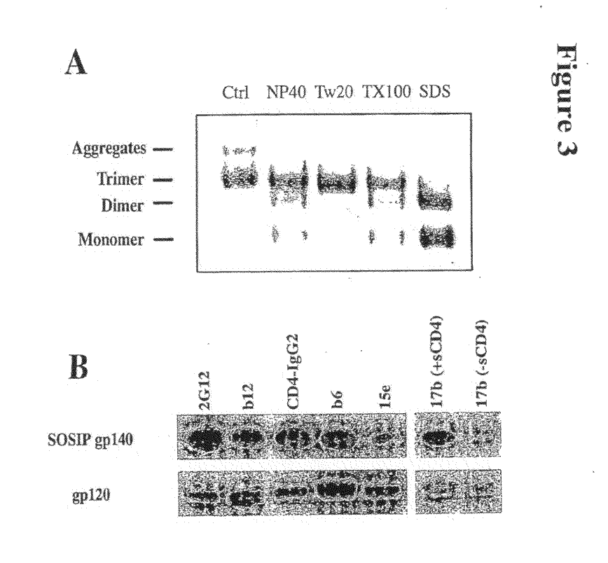

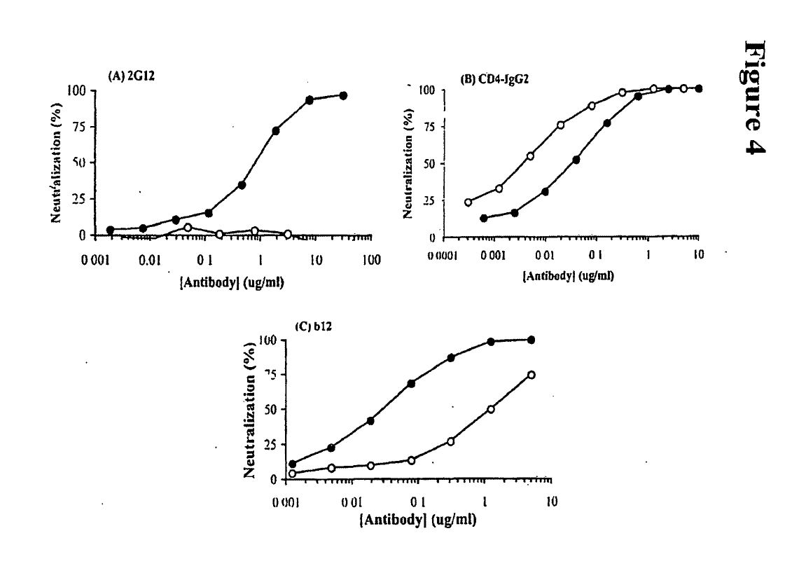

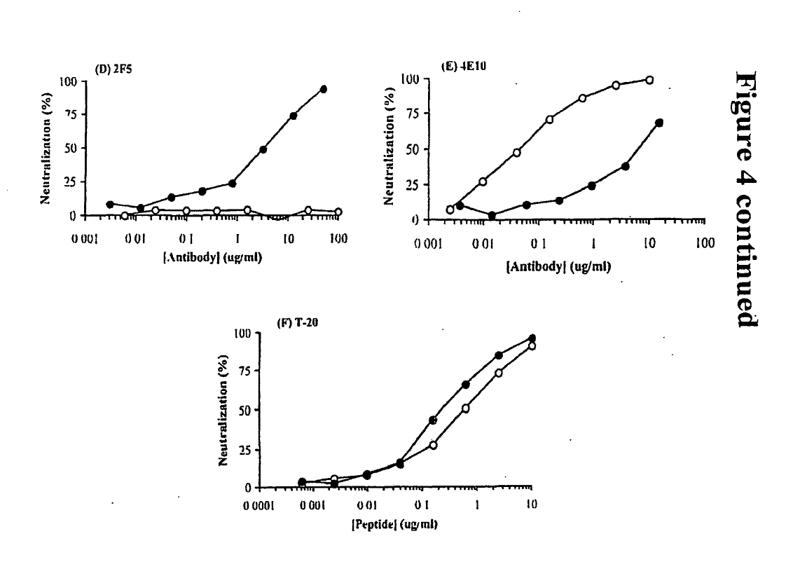

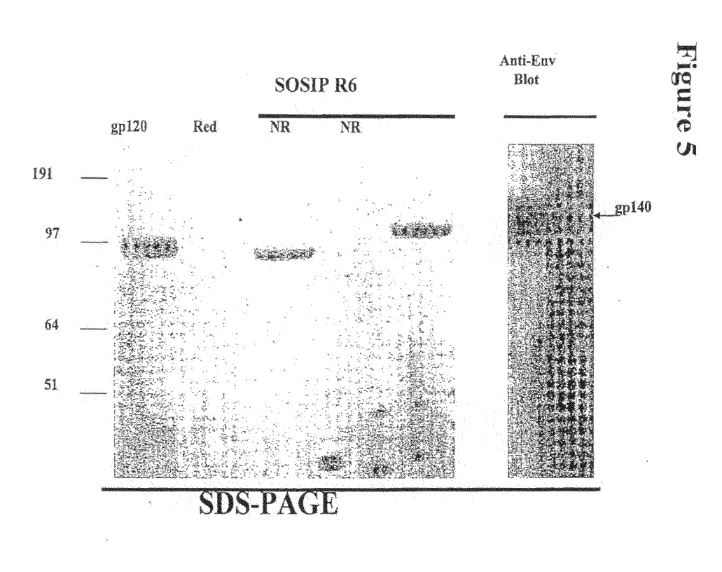

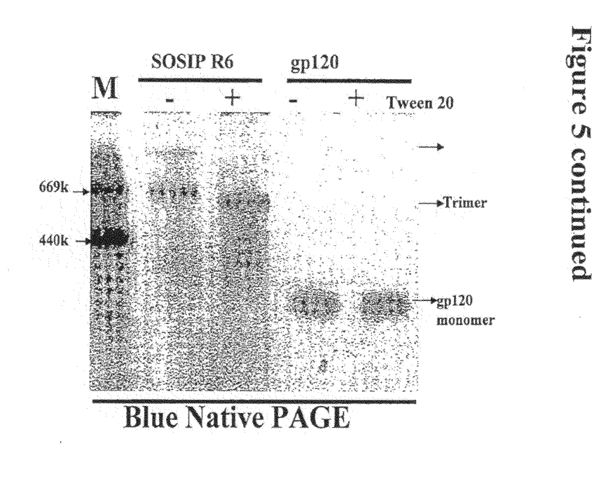

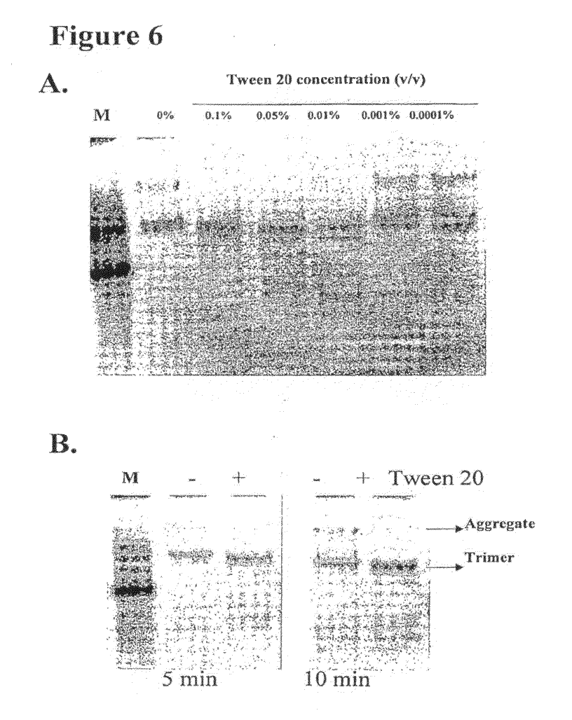

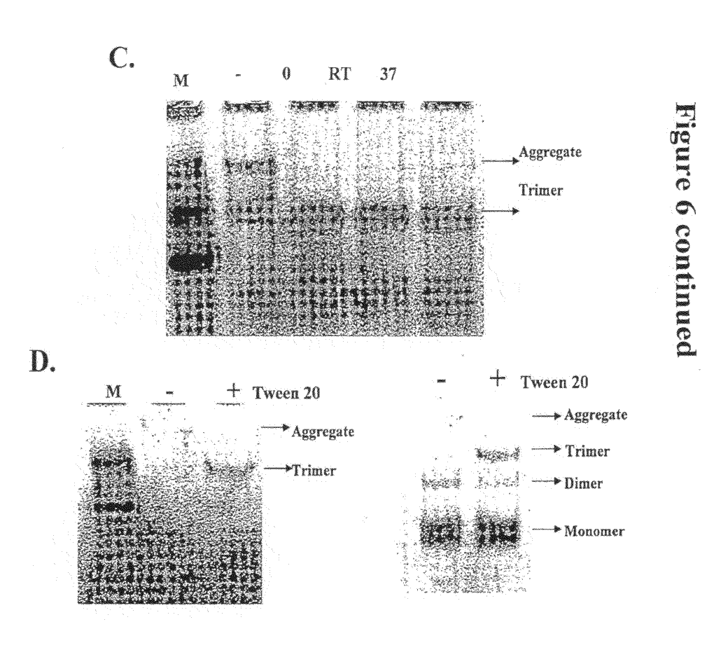

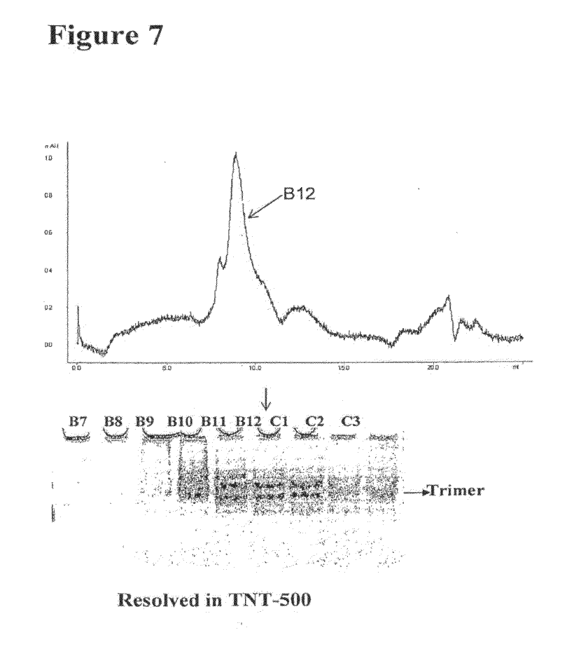

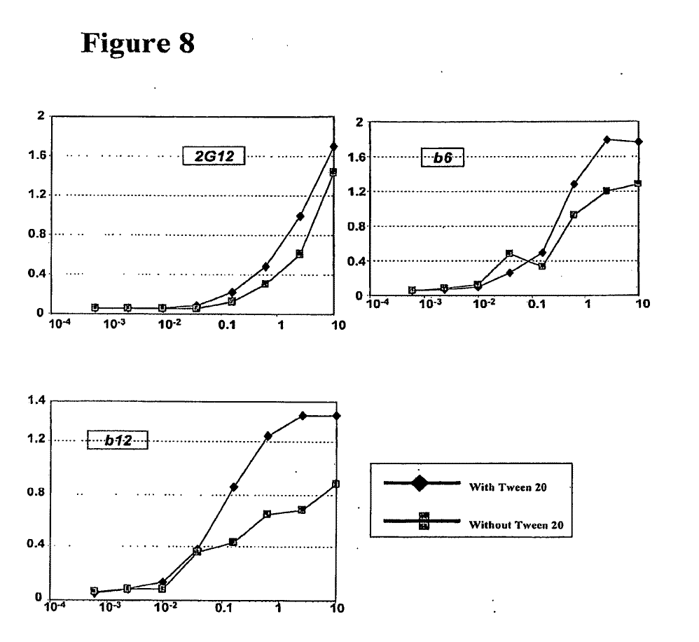

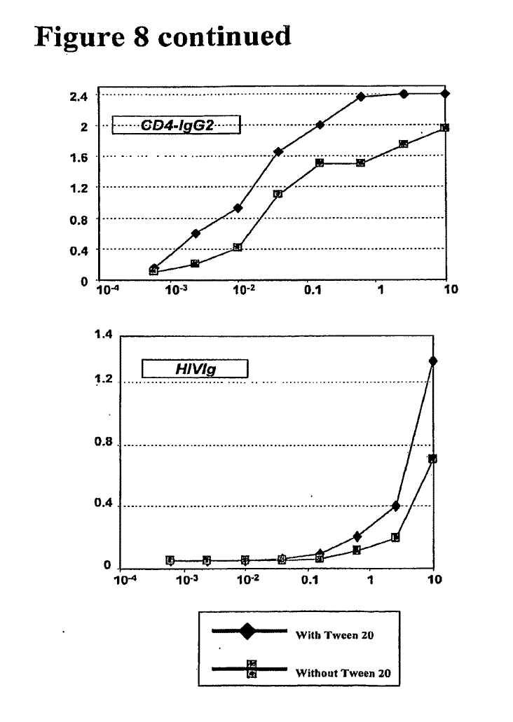

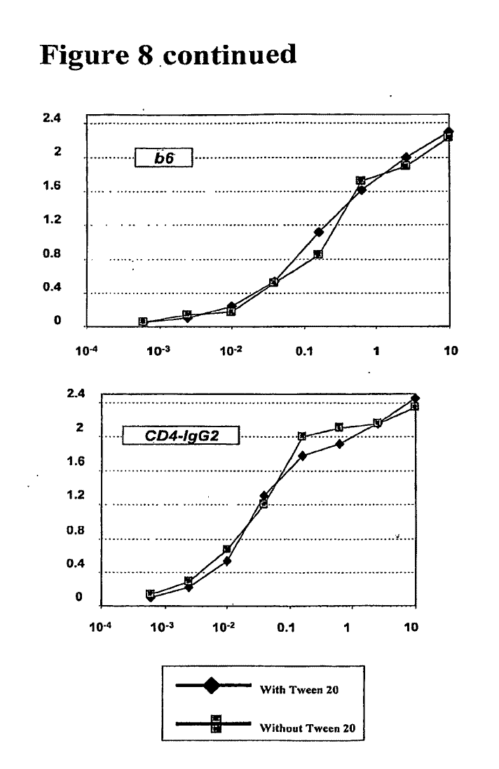

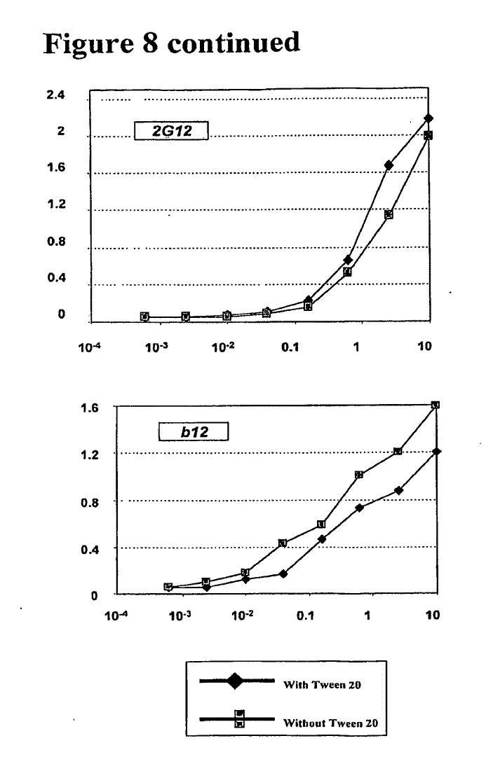

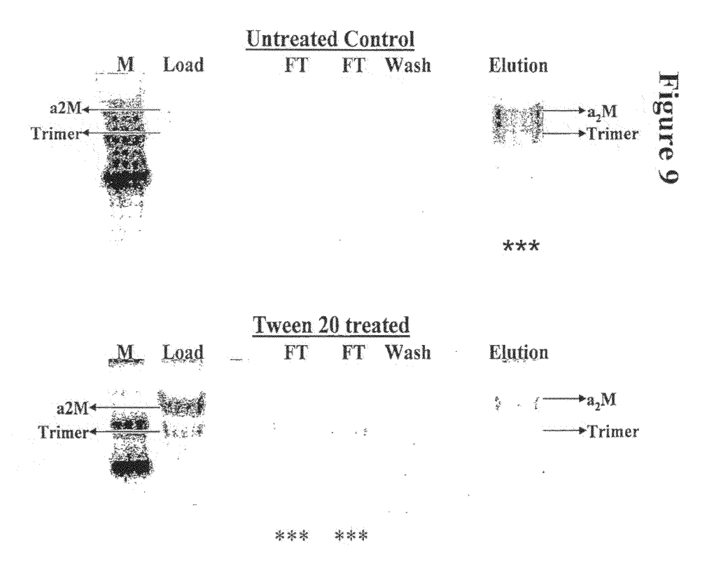

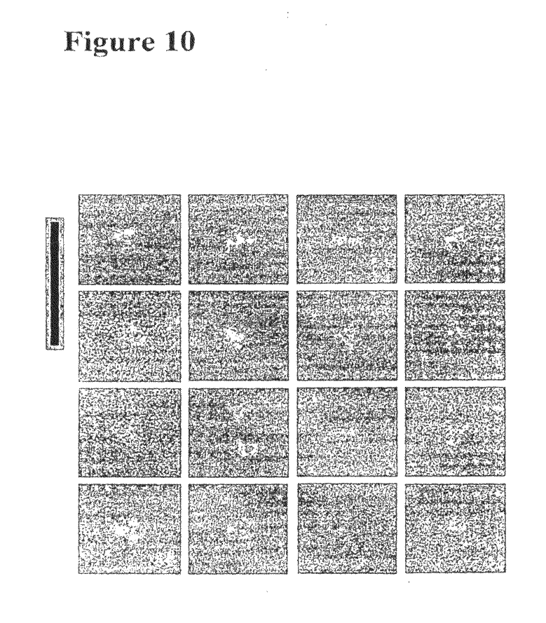

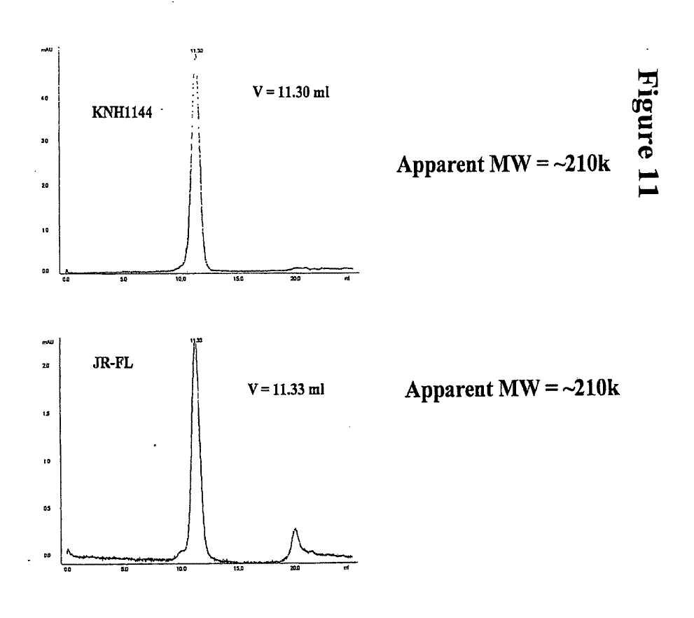

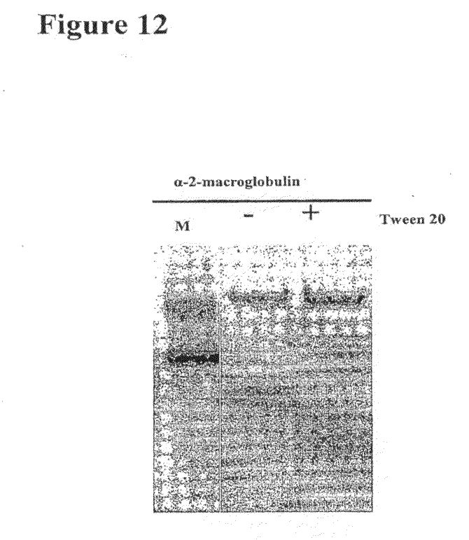

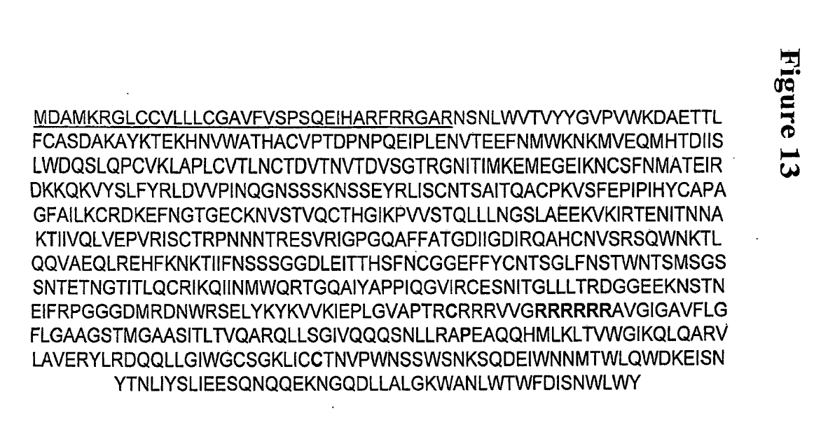

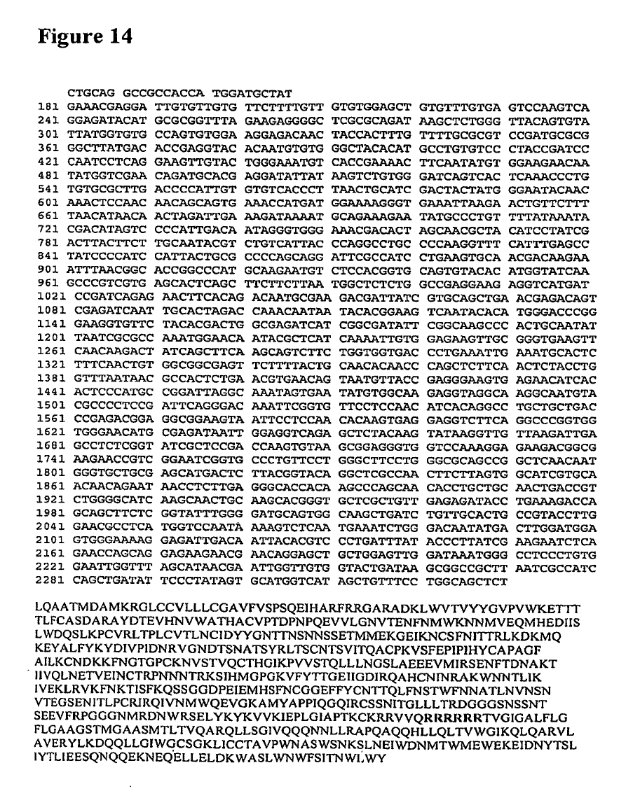

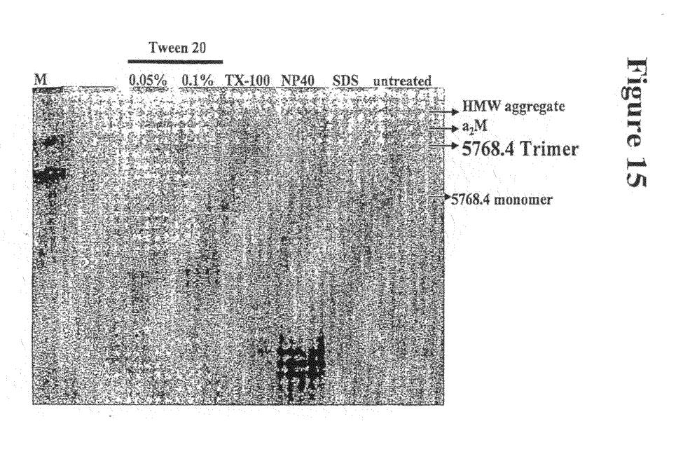

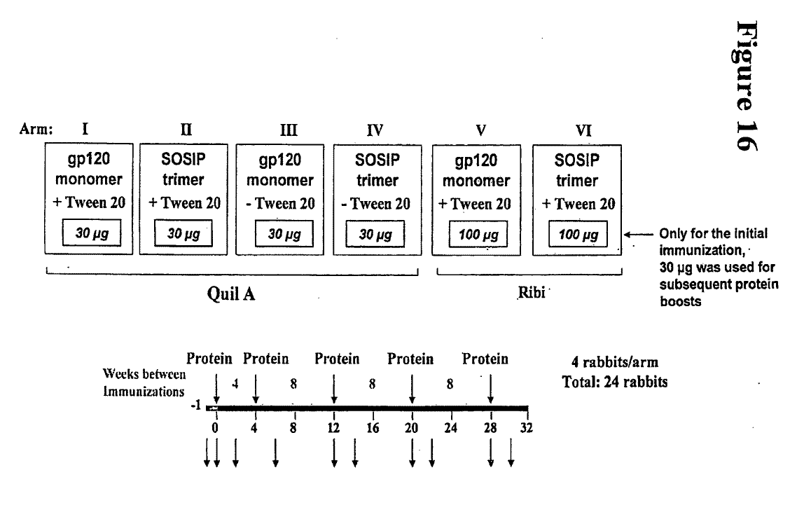

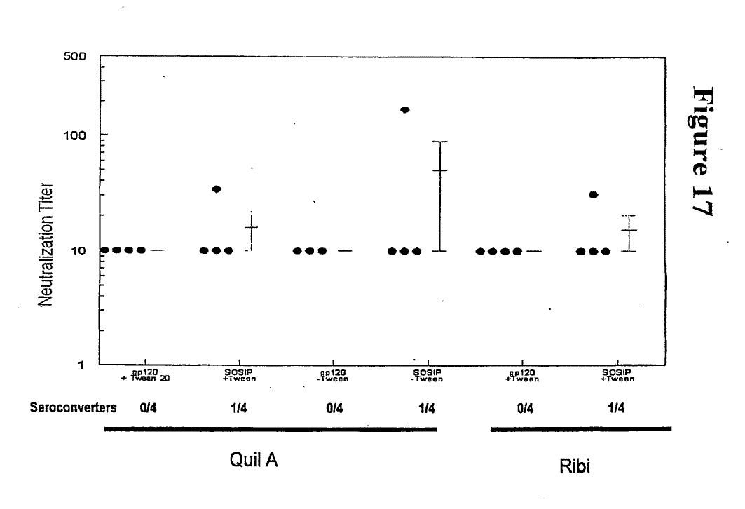

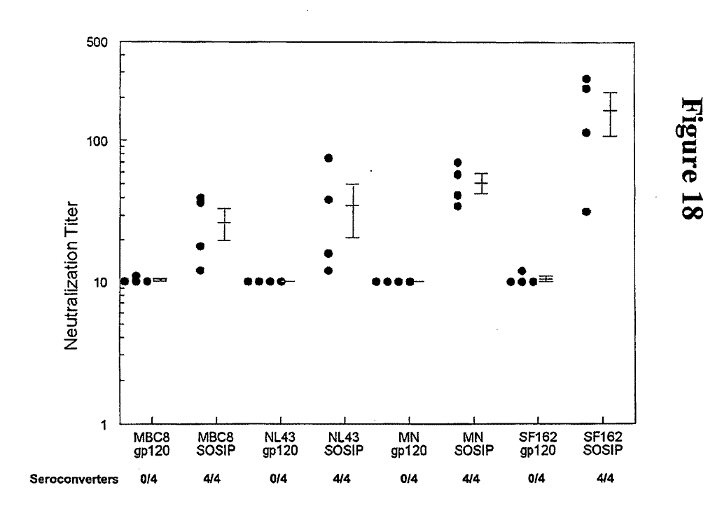

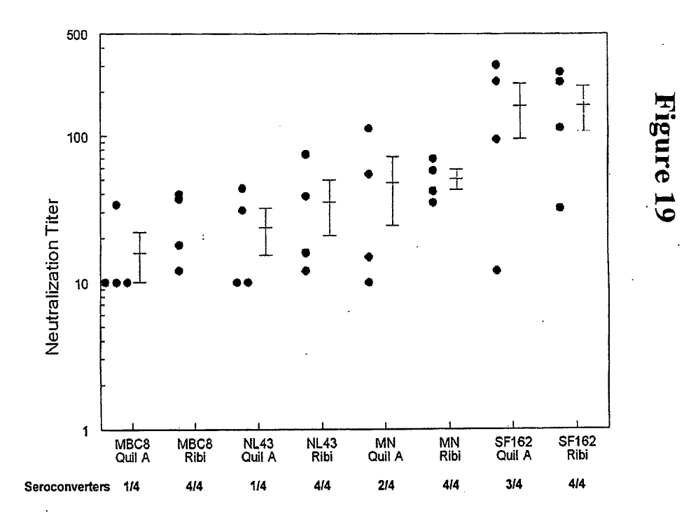

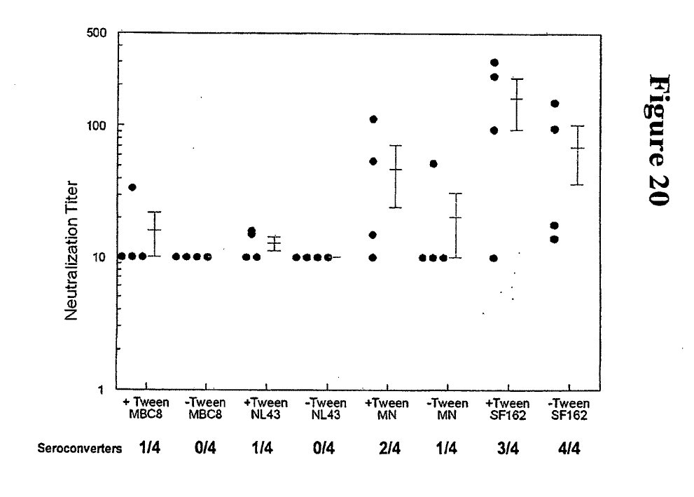

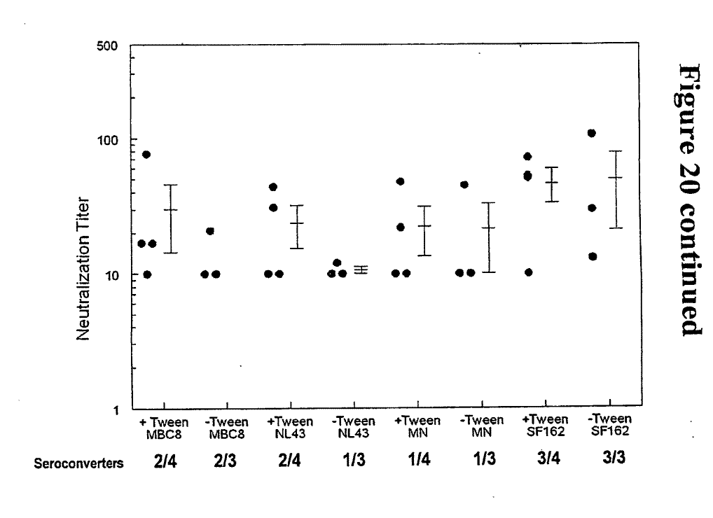

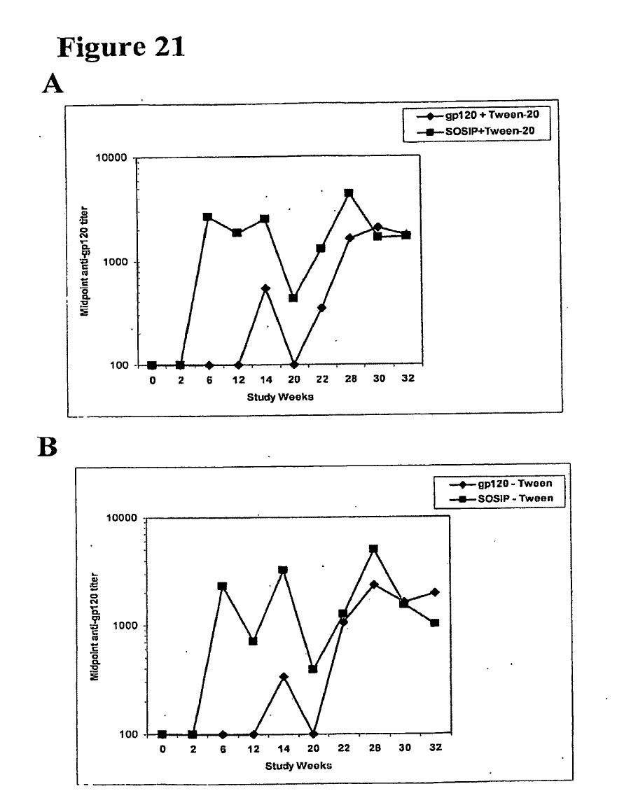

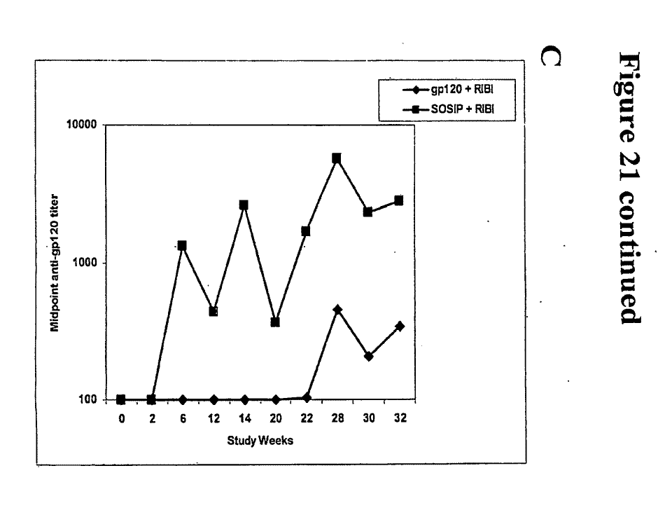

The extent of oligomer formation and cleavage in subtype A Env proteins was first assessed in small-scale studies using concentrated, unfractionated supernatants (10 ml) derived from transiently transfected 293T cells (FIGS. 1A and 1B). The oligomer contents of versions of SOS gp140 proteins containing (+) or lacking (−) the trimer-stabilizing I571P mutation” were analyzed by BN-PAGE (FIG. 1A). KER2008 SOS gp140 was expressed predominantly as a monomer. The introduction of the I559P substitution improved oligomer formation to an extent, but few SOSIP gp140 trimers could be detected. KNH1144 SOS gp140 was expressed as a dimer, while the corresponding SOSIP gp140 protein was predominantly trimeric. The other four env clones (KNH1207, KNH1211, BB359 and Q23-17) generated a mixture of monomers, dimers and trimers. The presence of the I571P substitution did not markedly improve the trimer content of the unpurified expression supernatants for these four clones (FIG. 1A).

The extent of SOSIP gp140 cleavage was assessed by reduced SDS-PAGE (FIG. 1B). The cleavage of KNH1144 SOSIP gp140 was increased from 46% (±9%; n=6) to 77% (±14%) by introducing the cleavage-enhancing Hex-Arg (R6) motif⁴³. Cleavage was increased to 87% (±15%) when Furin was co-expressed. The KNH1207 SOSIP gp140 protein was naturally very poorly cleaved (10%±6%; n=5); the extent was increased to 48% (±6%) by the R6 substitution, and to 79% (±12%) by Furin co-expression. The remaining SOSIP gp140 proteins (KER2008, KNH1211, BB359 and Q23-17) were also not fully cleaved, but again the extent of cleavage was substantially increased by Furin co-expression or by introducing the R6 motif (FIG. 1B). When the two techniques were combined, i.e., when Furin was co-expressed with the R6 mutant of KNH144 SOSIP gp140, the extent of Env cleavage approached 100% such that uncleaved proteins could not be detected.

Purification of Oligomeric SOSIP gp140 Proteins:

The initial, small-scale studies indicated that KNH1144 SOSIP gp140 was predominantly secreted as trimers (FIG. 1A). Most SOSIP gp140 proteins are expressed as a mixture of oligomeric forms, for example, as observed with the KNH1211, BB359 and Q23-17 clones (FIG. 1A). The clean separation on BN-PAGE gels of each individual Env band from the latter clones suggested that further purification to homogeneous trimers could be achieved by the use of SEC. To this end, the KNH1144 and KNH1211 SOSIP gp140 proteins were expressed on a larger (0.5-2 L) production scale. For comparison, KNH1144 SOS gp140 proteins were also prepared.

The various gp140 proteins were generated by transient transfection. Thereafter, the clarified and concentrated supernatants were fractionated by SEC^{40,45}. Samples from each eluted SEC fraction were analyzed by BN-PAGE; the gel profiles of the trimer, dimer and monomer peaks are shown in FIG. 2. Trimers could be detected in the KNH1211 SOSIP gp140 preparation, with little or no dimer contamination, but only at low abundance and only in a few SEC fractions; dimers and monomers were more abundant, but could be cleanly separated from the trimers by SEC. By contrast, the KNH1144 SOSIP gp140 was resolved as a nearly homogeneous trimeric species that was present in several fractions prior to and including those shown in FIG. 2. KNH1144 SOS gp140, which lacks the trimer-stabilizing I571P substitution, was expressed predominantly as dimers, with few trimers or monomers detectable.

Although KNH1211 SOSIP gp140 trimers could be purified, KNH1144 SOSIP gp140 were selected for further immunogenicity studies because among the Env proteins studied herein, the KNH1144 R6 SOSIP gp140 Env protein yielded the most abundant and purest trimers.

Biophysical and Antigenic Properties of Purified, Trimeric KNH1144 SOSIP gp140:

A purified Env trimer should meet several criteria to establish that it has been synthesized, folded, cleaved and otherwise post-translationally modified correctly. For example, it is important to establish that SOSIP gp140 trimers are oligomerized via non-covalent interactions between the gp41_{ECTO} subunits, and not via aberrant inter-subunit disulfide bonds^{40,66,67}. For these assessments, purified KNH1144 SOSIP gp140 trimers were treated for 1 h at 25° C. with 0.1% (v/v) ionic (SDS) and non-ionic (NP-40, Tween®-20, Triton X-100) detergents and then analyzed by BN-PAGE. The non-ionic detergents had little (NP-40 and Triton X-100) or no (Tween®-20) adverse effects on the trimers, while SDS caused them to completely dissociate into dimers and monomers (FIG. 3A). Hence, non-covalent bonds that can be readily disrupted by ionic detergents at room temperature hold these trimers together. This is consistent with studies of JR-FL SOSIP gp140 trimers⁴⁴.

To determine whether KNH1144 SOSIP gp140 trimers retained at least some of the complex, discontinuous neutralization epitopes present on gp120, the binding of an appropriate set of MAbs was studied. It was also desirable to ascertain whether the gp120 component of the trimer could undergo sCD4-induced conformational changes associated with increased exposure of the co-receptor binding site, which can be probed using MAb 17b. Analytical assessments of this nature are commonly used to assess the antigenic profiles of trimeric Env proteins^{19-22,40,44}.

The purified KNH1144 SOSIP gp140 trimer was immunoprecipitated by the neutralizing MAbs b12 and 2G12, and by the CD4-IgG2 protein, showing that its gp120 moiety expresses complex, discontinuous epitopes (FIG. 3B). Furthermore, sCD4 increased the binding of MAbs 17b and X5 to their CD41 epitopes on gp120 (FIG. 3B). Taken together, these data suggest that the gp120 component of the KNH1144 SOSIP gp140 trimer is properly folded and functional. However, MAbs b6 and 15e, directed against non-neutralizing epitopes associated with the CD4-binding site, were also reactive with KNH1144 SOSIP gp140 trimers, despite being unable to neutralize the corresponding Env-pseudotyped virus, HIV-1_{KNH1144} (FIG. 3B). Altering the Env conformation in such a way as to reduce the exposure of epitopes that bind non-neutralizing antibodies may be important for making better mimics of the truly native Env spikes present on infectious virions.³⁷ The limited number of antibodies reactive with subtype A Env proteins precluded further probing of the topology of KNH1144 SOSIP gp140 trimers (see below). The gp41 MAbs 2F5 and 4E10 did not precipitate detectable levels of KNH1144 SOSIP gp140. The KNH1144 SOSIP gp140 sequence contains a polymorphism in the 2F5 core motif (ALGKWA) that is probably sufficient to preclude recognition by this MAb. However, the core motif for 4E10 (WFDI) is present in KNH1144 SOSIP gp140⁶⁸.

Antigenic Properties of Subtype A gp120 Proteins:

To further study the antigenicity of subtype A Env proteins, monomeric gp120 proteins were produced from all 6 clones, and then their reactivity with MAbs was tested by ELISA. For comparison, two subtype B gp120 Env polypeptidess (JR-FL and BaL), and one Env polypeptide from subtype C (DU151) were also included. The gp120s were captured onto a plastic surface by the immobilized, CS-directed antibody D7324, as in previous studies of subtype B and non-subtype B gp120s^{29,63}. The binding of a saturating concentration of CD4-IgG2 was used to normalize the input amount of the different gp120 proteins; in other words, the volume of different culture supernatants added to the ELISA wells was varied to yield approximately equivalent binding of CD4-IgG2 in each case. The binding of the test MAbs was then assessed using the calibrated amount of each gp120.

The half-maximal binding concentrations for CD4-IgG2 against the subtype A gp120s (mean 135±87 ng/ml, range 63-255 ng/ml) were similar to those for the subtype B gp120s, JR-FL (46 ng/ml) and BaL (57 ng/ml) (Table 1). The KER2008 (236 ng/ml) and BB359 (255 ng/ml) gp120s had the lowest apparent affinities for CD4-IgG2 among the test panel; KNH1207 (68 ng/ml) and KNH1211 (63 ng/ml) gp120s had the highest affinities. These results confirm that the CD4 binding site was folded appropriately on each of the gp120s.

TABLE 1
Antibody binding to gp120s derived from Env subtypes A to C

| | | Half-maximal binding (μg/ml) of indicated antibody reagent to gp120 ^{a} | | | | | |
|---|---|---|---|---|---|---|---|
| Env subtype | gp120 | 2G12 | CD4-IgG2 | HIVIg | b12 | PA1 | 3119 |
| A | KER 2008 | 0.064 | 0.236 | 2.230 | >1 | >9 | 1.569 |
| | KNH 1144 | >1 | 0.103 | 2.213 | 0.642 | >9 | 0.636 |
| | KNH 1207 | 0.478 | 0.068 | 1.693 | 0.092 | >9 | 0.337 |
| | KNH 1211 | >1 | 0.063 | 2.629 | 0.033 | >9 | 0.176 |
| | B2539 | >1 | 0.255 | 0.651 | >1 | >9 | 0.406 |
| | Q23-17 | 0.540 | 0.083 | 1.834 | >1 | >9 | 0.094 |
| B | JR-FL | 0.061 | 0.046 | 0.265 | 0.027 | 0.215 | >9 |
| | BaL | 0.012 | 0.057 | 0.046 | 0.015 | 0.009 | 0.059 |
| C | DU151 | >1 | 0.090 | 3.653 | 0.018 | >9 | 0.043 |

^{a} Half-maximal (50%) antibody binding concentration (μg/ml) to indicated gp120, by ELISA. A value of >1or >9 indicates that half-maximal binding was not achieved at this antibody concentration. Data presented are the mean half-maximal binding concentrations determined from at least three independent experiments.

MAbs 2G12 and b12, the polyclonal HIVIg (subtype B) preparation and the type-specific MAbs, PA1 and CA13 were more variable in their binding profiles. Three of the subtype A gp120s (KNH1144, KNH1211 and BB359) and the subtype C gp120 (DU151) failed to bind 2G12, while three subtype A gp120s (KER2008, Q23-17 and BB359) did not react with b12. The half-maximal binding concentrations of HIVIg (subtype B) against the subtype A gp120s were, in most cases, ˜10-fold higher than those for the subtype B gp120s, as expected from an earlier study.⁶⁹. The anti-V3_{JR-FL} MAb PA1 failed to bind any of the non-subtype B gp120s, which was expected given the extent of V3 sequence variation.^{68,70,71}. MAb CA13 reacted efficiently with some subtype A gp120s (KNH1211, Q23-17) and also with the subtype C gp120 DU151, but bound less well to the subtype A gp120s, particularly the KER2008 and KNH1144 proteins. The epitope for CA13 has not been defined, although it is probably continuous, given that the antibody binds denatured gp120 (FIG. 1B). CA13 reacted poorly with HIV-1_{JR-FL} gp120, but bound efficiently to gp120 from the other subtype B strain, HIV-1_{BaL}.

The gp120 binding activities of rabbit sera that had been generated against monomeric gp120 proteins from subtypes A (HIV-1_{BB359} and HIV-1_{Q23-17}), B (HIV-1_{JR-FL} and HIV-1_{BaL}) and C (HIV-1_{DU422} and HIV-1_{DU179}) (Doms R. W. and Puffer B., unpublished) were next examined. As expected, an examination of midpoint binding titers for each antiserum-gp120 pair revealed no definitive patterns indicative of subtype-specific reactivity (Table 2).⁶³. There were a few notable exceptions, however. Generally, the antisera to BB359 (subtype A) and JR-FL (B) gp120s reacted preferentially with subtype A and B gp120s, respectively. This was not the case, however, for the sera raised against Q23-17 (subtype A) or BaL (B) gp120. Antisera to the BB359, Q23-17 and JR-FL gp120s bound more efficiently to the homologous gp120 than to the heterologous proteins. Overall, sera elicited to a gp120 from a particular Env subtype neither exclusively nor preferentially bound to gp120s derived from the same Env subtype,

TABLE 2
Seroreactivity of Env subtype-specific rabbit antisera

| | | Midpoint binding titers of antisera raised to the indicated gp120 ^{a} | | | | | |
|---|---|---|---|---|---|---|---|
| Env | | Subtype A | | Subtype B | | Subtype C | |
| subtype | gp120 | B2539 | Q23-17 | JR-FL | BaL | DU123 | DU179 |
| A | KER 2008 | 881 | 1232 | 1727 | 370 | 1801 | 102 |
| | KNH 1144 | 3753 | 3632 | 1557 | 1042 | 705 | 248 |
| | KNH 1207 | 26279 | 9890 | 11493 | 9745 | 12370 | 307 |
| | KNH 1211 | 17026 | 9929 | 1885 | 1056 | 703 | 453 |
| | B2539 | 26990 | 7028 | 1383 | 1633 | 2176 | 208 |
| | Q23-17 | 23884 | 13013 | 6950 | 5942 | 11581 | 571 |
| B | JR-FL | 6250 | 5215 | 25753 | 1148 | 4415 | <100 |
| | BaL | 11261 | 10944 | 24051 | 3347 | 15720 | 1082 |
| C | DU151 | 7138 | 4498 | 5348 | 3763 | 10257 | 248 |

^{a} Midpoint (50%) binding titers of Env subtype-specific pooled antisera against indicated gp120, by ELISA. Each pool comprises sera from three rabbits immunized with indicated gp120: subtype A (B2539, Q23-17), B (JR-FL, BaL) and C (DU422, DU179). Autologous gp120-serum pairs are indicated in bold. A value of <100 indicates midpoint binding was not achieved at this serum dilution. Data presented are the mean titers determined from 2-3 independent experiments.

The serum reactivity of KNH1144 gp120 was ranked among the subtype A gp120 panel according to the midpoint binding titer for each anti-gp120 serum pool tested. In most cases, KNH1144 gp120 was the fifth most seroreactive of the six subtype A gp120s (Table 2). Overall, KNH1144 gp120 has a relatively low reactivity with both MAbs (Table 1) and anti-gp120 antisera (Table 2), although both sets of serological reagents are limited in scope.

Neutralization Sensitivity of Subtype A Env-Pseudotyped HIV-1:

To assess the neutralization sensitivity of viruses bearing subtype A Env proteins, cytoplasmic tail-truncated gp140s from KER2008, KNH1144, KNH1207 and KNH1211 were co-expressed in trans with the pNL4/3.Luc plasmid to form single cycle, replication-incompetent pseudoviruses⁷². Pseudovirions bearing the full-length Env protein from HIV-1_{JR-FL} were also studied, for comparison.

A test panel of antibodies reported to reproducibly neutralize subtype B and non-subtype B viruses in many published studies was selected. Some of the antibodies used for the gp120-binding assessments were included.²⁷⁻³⁰. The tetrameric CD4-IgG2 protein neutralized all four subtype A Env-pseudotyped viruses with similar potencies (IC₅₀ 0.04-0.100 μg/ml; IC₉₀ 0.5-1.45 μg/ml). This was not surprising given that that CD4-IgG2 recognizes the actual CD4 binding site and is less affected by the sequence variation that influences the overlapping epitopes for CD4bs-directed MAbs.²⁹. Neutralization by CD4-IgG2 provided a frame of reference for the sensitivity or resistance of the same Env-pseudotyped viruses to the other test reagents.

MAb b12 neutralized all of the subtype A Env-pseudotyped viruses by 50% (IC₅₀ 0.21-2.33 μg/ml), but failed to neutralize HIV-1_{KER2008} and HIV-1_{KNH1144} by 90% at the highest concentration tested (3 μg/ml). Of the remaining two subtype A Env-pseudotyped viruses, HIV-1_{KNH1207} was more sensitive (IC₅₀ 0.21 μg/ml; IC₉₀ 0.90 μg/ml) than HIV-1_{KNH1211} (IC₅₀ 1.14 μg/ml; IC₉₀ 1.90 μg/ml).

Whether 2F5 could neutralize these Env-pseudotype viruses was largely predictable by the amino acid sequence of its canonical gp41 epitope^{29,68,73}. Thus, the HIV-1_{KER2008} (epitope sequence, ALDKWS (SEQ ID NO:4), and HIV-1_{KNH1144} (ALGKWA)(SEQ ID NO:5) Env-pseudotyped viruses were not neutralized by 2F5 (IC₅₀ >3 μg/ml), mostly likely due to a single amino acid change within the antibody binding site (underscored). HIV-1_{KNH1211}, (ALDKWA (SEQ ID NO:6); IC₅₀ 0.03 μg/ml; IC₉₀ 0.99 μg/ml) and HIV-1_{KNH1207}, (ALDKWA; IC₅₀ 0.01 μg/ml; IC₉₀ 0.21 μg/ml) were neutralized by 2F5 at relatively low concentrations^{29,68,73}.

The development and characterization of subtype A Env proteins involved a comparison of the antigenicity and immunogenicity of soluble, cleaved SOSIP gp140 trimers as well as the neutralization sensitivity of Env-pseudotyped viruses derived from the subtype A strains. Following these initial characterizations, it was determined that KNH1144 SOSIP gp140 had the most suitable properties at the protein level, as outlined above. Following from these assessments, the neutralization sensitivity of pseudoviruses bearing the KNH1144 Env was compared with the neutralization sensitivity of pseudoviruses bearing the subtype 13 Env, JR-FL.⁴⁵. As the subtype A env clones were available as truncated gp140 genes, the differing lengths of the gp41 cytoplasmic tail in the two Env-pseudotyped viruses (truncated in KNH1144, full-length in JR-FL) could have minor quantitative and/or qualitative effects on entry inhibition.⁷⁴⁻⁴⁶. The possibility that such truncation could have a significant impact on the neutralization sensitivity of the KNH1144 Env-pseudotyped virus was not ruled out, and such effects were considered to likely be small compared to those that could be created by subtype-dependent sequence variation.

Inhibition of the Env-pseudotyped viruses by CD4-IgG2, b12, 2G12, 2F5 and 4E10 and the T-20 fusion inhibitory peptide is shown in Table 3. HIV-1_{KNH1144} was more sensitive than HIV-1_{JR-FL} to CD4-IgG2, but less sensitive to MAb b12. The reduced activity of b12 against HIV-1_{KNH1144} is consistent with its lower apparent affinity for KNH1144 gp120 in ELISA (Table 1). HIV-1_{KNH1144} was not neutralized by 2G12, again consistent with the non-reactivity of this MAb with KNH1144 gp120. As noted above, HIV-1_{KNH1144} was resistant to neutralization by MAb 2F5, but it was ˜100-fold more sensitive than HIV-1_{JR-FL} to 4E10. HIV-1_{KNH1144} was −10-fold less sensitive than HIV-1_{JR-FL} to neutralization by the polyclonal HIV+ human serum, LTNP-2, but the neutralization titers for another polyclonal HIV+ serum, FDA#2, were similar for each virus (Table 3). Both sera were from individuals infected with subtype B viruses, implying that the neutralizing antibodies (Nabs) present are not subtype-restricted. The fusion inhibitor T-20 inhibited both test viruses with equally sensitivity. HIV-1_{KNH1144} was generally more sensitive than HIV-1_{JR-FL} to an anti-CCR5 MAb (PA14) and to CCR5-directed small molecule entry inhibitors (SCH-C, SCH-D) (Table 3). AZT, which served as a control for the amount and/or relative infectivity of the two Env-pseudotyped viruses, inhibited both of them with equal potency.

TABLE 3
Neutralization of HIV-1 pseudotyped with subtype A (KNH1144) or
B (JR-FL) Env^{a}

| | | Env-pseudotyped | |
|---|---|---|---|
| | | neutralization | |
| Class of Inhibitor | Reagent | KNH1144 | JR-FL |
| Anti-Env antibodies (μg/ml) | 2G12 | >50 | 0.95 |
| | CD4-IgG2 | 0.003 | 0.05 |
| | b12 | 1.5 | 0.04 |
| | 2F5 | >100 | 4 |
| | 4E10 | 0.06 | 10 |
| Polyclonal HIV+ sera (dilution) | LTNP-2 | 1:100 | 1:1500 |
| | FDA#2 | 1:50 | 1:100 |
| Peptidic fusion inhibitor (ng/ml) | T-20 | 800 | 250 |
| CCR5 inhibitor (nM) | PA14 | 40 | 100 |
| | SCH-C | 0.04 | 0.2 |
| | SCH-D | 0.01 | 0.1 |
| RT inhibitor (nM) | AZT | 240 | 220 |

^{a}Neutralization (50% endpoint) of the indicated Env-pseudotyped HIV-1 by anti-Env antibodies (in μg/ml), polyclonal HIV+ human sera (serum dilution), a fusion inhibitor (in ng/ml), CCR5 entry inhibitors (in nM) and the RT inhibitor, AZT (in nM).

Discussion

How to make an Env-based subunit immunogen that can elicit antibodies capable of broadly neutralizing primary HIV-1 strains in vitro is central to current vaccine development strategies. The present invention describes the generation of a stabilized, cleaved, soluble trimeric form of gp140 derived from a recent subtype A sequence isolate, KNH1144, from sub-Saharan Africa. The present invention encompasses stabilized trimeric Env proteins as immunogens that may be superior to other forms of HIV-1 Env.

The panel of subtype A env genes was derived from recent, representative isolates from Kenya.^{5,14,46,47}. Six SOS, SOSIP and SOSIP.R6 gp140 protein expression vectors based on these sequences were successfully engineered. The extent of Env cleavage by naturally occurring cellular endoproteases varied, although in most cases full cleavage could be achieved if the cleavage-enhancing R6 motif was introduced and/or if the proteins were co-expressed with the furin endoprotease. The efficiency of trimer formation was also variable, ranging from negligible (KER2008) to substantial (KNH1144). The oligomer and/or aggregate content of different uncleaved gp140 proteins, including those based on non-subtype B Env templates, has also been found to be very variable in a way that cannot be predicted from the protein sequences.^{17,18,20,22,40,66}.

SOS and SOSIP gp140 proteins derived from KNH1211 and KNH1144 gp140 were selected for scale-up and fractionation by SEC. While KNH1211 SOSIP gp140 trimers were stable enough to survive the column chromatography procedures, those from KNH1144 SOSIP gp140 were much more stable and abundant. Furthermore, KNH1144 SOSIP gp140 formed almost homogeneous trimers that withstood purification, which is of obvious advantage for larger-scale production efforts. The trimer content of KNH1144 SOSIP gp140 is superior to other Env protein preparations.

Immunoprecipitation analysis showed that the trimeric KNH1144 SOSIP gp140 protein binds neutralizing and non-neutralizing MAbs directed against the CD4 binding site. It also undergoes a sCD4-induced conformational change that increased the exposure of the 17b epitope overlapping the CCR5 binding site.^{50,51}. These trimers were also relatively resistant to non-ionic detergents (Tween®-20, NP-40 and Triton X-100), but the ionic detergent, SDS, was able to dissociate the trimers into dimers and monomers at room temperature. Thus, the interactions between the gp41_{ECTO} subunits are non-covalent in nature, similar to the results seen for JR-FL SOSIP gp140 trimers.⁴⁴. Taken together, these antigenic and biophysical properties suggest that the KNH1144 SOSIP gp140 protein forms properly associated Env trimers.

There are conformational differences between the soluble gp140 trimers of the present invention and the Env forms present on infectious virions. For example, non-neutralizing CD4bs MAbs do efficiently bind to the soluble trimers, while they fail to bind to functional spikes on virions. Whether such differences are common among all soluble gp140 trimers, perhaps because of the absence of the transmembrane and internal regions of gp41, or whether the explanation lies elsewhere, e.g., due to a minor proportion of misfolded Env trimers, remains to be determined. Nonetheless, if the exposure of non-neutralizing CD4bs-associated epitopes on soluble trimers should adversely influence their immunogenicity, additional modifications to the protein conformation could be made.³⁷.

The antigenic properties of KNH1144 Env were further characterized, compared both to the other subtype A Envs and to Envs from subtypes B and C. The antibody-binding profiles of monomeric gp120s and trimeric gp140s was assessed, along with the neutralization sensitivity of the corresponding Env-pseudotyped viruses. Using the ELISA technique, it was found that KNH1144 gp120 bound rabbit anti-gp120 sera and certain MAbs, including b12 and 2G12, less well than most of the other subtype A gp120s.

The 2G12 epitope is considered to include N-linked glycans at positions N332 and N392, with some minor influence from the glycans present on residues N295, N339 and N386⁷⁷. The non-reactivity of 2G12 with KNH1144 and BB359 gp120s by ELISA is consistent with the absence of N-linked glycosylation sites at position N295. The DU151 and KNH1211 gp120s have multiple substitutions at positions N295, N339, N386 and/or N392 that either disrupt the glycan sites entirely, or shift their locations by one or two residues. The reduced binding of 2G12 to Q23-17 and KNH1207 gp120s might be explained by the disruption of the N295 motif (Q23-17) and the N339/N386 motifs (KNH1207). Thus, at least for some of these gp120s, the binding of MAb 2G12 could be predicted from sequence analysis. However, although 2G12 was non-reactive with KNH1144 gp120 in an ELISA and could not neutralize the Env-pseudotyped virus, this MAb did successfully immunoprecipitate both KNH1144 gp120 and SOSIP gp140. These data suggest that the presentation of the putative MAb 2G12 binding site on gp140 SOSIP is somewhat different from its presentation on monomeric gp120 or on the surface of infectious virions. The reactivity of MAbs to KNH1144 gp120 and SOSIP gp140, such as 2G12 and others, with the surface topology of these novel trimers can be used to further characterize these Env proteins.

The HIV-1_{KNH144} Env-pseudotyped virus was significantly more sensitive than HIV-1_{JR-FL} to neutralization by the broadly reactive MAb, 4E10, but in contrast to HIV-1_{JR-FL,} it was not neutralized by MAb 2F5. This pattern of responses to 2F5 is consistent with the sequence variation within this MAb epitope on the two viruses. Thus, the KNH1144 sequence, ALGKWA (SEQ ID NO:5), is incompatible with known requirements for 2F5 binding, while the JR-FL sequence ELDKWA (SEQ ID NO:7). is representative of the canonical 2F5 epitope⁶⁸. However, the different responses of the two viruses to 4E10 (core epitope WFxxxxxxW; where x represents any amino acid)⁷⁸ cannot be explained simply by inspection of the corresponding sequences, WFDISNWLW (SEQ ID NO:8) for KNH114 and WFDITKWLW (SEQ ID NO:9) for JR-FL. The identity of the amino acids present within and around the canonical epitope for 4E10 is known not to predict the sensitivity of pseudoviruses to neutralization by this MAb⁶⁸. Furthermore, despite being able to efficiently neutralize the HIV-1_{KNH1144} Env-pseudotyped virus, MAb 4E10 does not immunoprecipitate the corresponding SOSIP gp140 trimers. Thus, the 4E10 epitope may not be properly accessible on KNH1144 SOSIP gp140 trimers. This is not the case for JR-FL SOSIP gp140 trimers because 4E10 immunoprecipitates these proteins efficiently. The lack of reactivity of 4E10 with KNH1144 SOSIP gp140 trimers may relate to how the 4E10 epitope is configured and presented in different settings49,68,78,79.

Overall, KNH1144 Env appeared to be less antibody-reactive than JR-FL Env. One interpretation of this finding is that the KNH1144 Env protein has relatively poorly exposed antibody-binding sites in general, although antibody recognition of gp120 proteins rarely reflects what happens with the native trimer⁸⁰. Alternatively, the observations may simply reflect how few antibody reagents are available for probing the topology of subtype A Env proteins.

In accordance with the present invention, a soluble, cleaved, trimeric form of Env based on a recent East African subtype A strain HIV-1_{KNH1144} was successfully generated. In vitro measurements of antibody reactivity were successfully carried out. Whether KNH1144 SOSIP gp140 trimers are superior immunogens to their JR-FL counterparts, particularly in terms of their ability to induce potent neutralizing antibodies against homologous and heterologous strains of HIV, may be empirically determined⁴⁵. To this end, purified KNH1144 SOSIP gp140 trimers were prepared and used in immunogenicity studies in rabbits.

### REFERENCES FOR EXPERIMENTAL DETAILS I

1 McCutchan F E: Understanding the genetic diversity of HIV-1. AIDS 2000; 14 Suppl 3: S31-S44.
2 Robertson D L, Anderson J P, Bradac J A, et al.: HIV-1 nomenclature proposal. Science 2000; 288: 55-56.
3 Perrin L, Kaiser L, and Yerly S: Travel and the spread of HIV-1 genetic variants. Lancet Infect Dis 2003; 3: 22-27.
4 UNAIDS: AIDS Epidemic Update 2004.
5 Dowling W E, Kim B, Mason C J, et al.: Forty-one near full-length HIV-1 sequences from Kenya reveal an epidemic of subtype A and A-containing recombinants. AIDS 2002; 16: 1809-1820.
6 Yang C, Li M, Shi Y P, et al.: Genetic diversity and high proportion of intersubtype recombinants among HIV type 1-infected pregnant women in Kisumu, western Kenya. AIDS Res Hum Retroviruses 2004; 20: 565-574.
7 Neilson J R, John G C, Carr J K, et al.: Subtypes of human immunodeficiency virus type 1 and disease stage among women in Nairobi, Kenya. J Virol 1999; 73: 4393-4403.
8 Louwagie J, Janssens W, Mascola J, et al.: Genetic diversity of the envelope glycoprotein from human immunodeficiency virus type 1 isolates of African origin. J Virol 1995; 69: 263-271.
9 Desrosiers R C: Prospects for an AIDS vaccine. Nat Med 2004; 10: 221-223.
10 Garber D A, Silvestri G, and Feinberg M B: Prospects for an AIDS vaccine: three big questions, no easy answers. Lancet Infect Dis 2004; 4: 397-413.
11 Graham B S: Clinical trials of HIV vaccines. Annu Rev Med 2002; 53: 207-221.
12 Klausner R D, Fauci A S, Corey L, et al.: The need for a global HIV vaccine enterprise. Science 2003; 300: 2036-2039.
13 McMichael A J, and Hanke T: HIV vaccines 1983-2003. Nat Med 2003; 9: 874-880.
14 Brown B K, Darden J M, Tovanabutra S, et al.: Biologic and genetic characterization of a panel of 60 human immunodeficiency virus type 1 isolates, representing clades A, B, C, D, CRF01_AE, and CRF02_AG, for the development and assessment of candidate vaccines. J Virol 2005; 79: 6089-6101.
15 Gao F, Weaver E A, Lu Z, et al.: Antigenicity and immunogenicity of a synthetic human immunodeficiency virus type 1 group m consensus envelope glycoprotein. J Virol 2005; 79: 1154-1163.
16 Gaschen B, Taylor J, Yusim K, et al.: Diversity considerations in HIV-1 vaccine selection. Science 2002; 296: 2354-2360.
17 Jeffs S A, Goriup S, Kebble B, et al.: Expression and characterisation of recombinant oligomeric envelope glycoproteins derived from primary isolates of HIV-1. Vaccine 2004; 22: 1032-1046.
18 Chakrabarti B K, Kong W P, Wu B Y, et al.: Modifications of the human immunodeficiency virus envelope glycoprotein enhance immunogenicity for genetic immunization. J Virol 2002; 76: 5357-5368.
19 Srivastava I K, Stamatatos L, Legg H, et al.: Purification and characterization of oligomeric envelope glycoprotein from a primary R5 subtype B human immunodeficiency virus. J Virol 2002; 76: 2835-2847.
20 Yang X, Florin L, Farzan M, et al.: Modifications that stabilize human immunodeficiency virus envelope glycoprotein trimers in solution. J Virol 2000; 74: 4746-4754.
21 Yang X, Lee J, Mahony E M, et al.: Highly stable trimers formed by human immunodeficiency virus type 1 envelope glycoproteins fused with the trimeric motif of T4 bacteriophage fibritin. J Virol 2002; 76: 4634-4642.
22 Zhang C W, Chishti Y, Hussey R E, and Reinherz E L: Expression, purification, and characterization of recombinant HIV gp140. The gp41 ectodomain of HIV or simian immunodeficiency virus is sufficient to maintain the retroviral envelope glycoprotein as a trimer. J Biol Chem 2001; 276: 39577-39585.
23 Binley J M, Sanders R W, Clas B, et al.: A recombinant human immunodeficiency virus type 1 envelope glycoprotein complex stabilized by an intermolecular disulfide bond between the gp120 and gp41 subunits is an antigenic mimic of the trimeric virion-associated structure. J Virol 2000; 74: 627-643.
24 Gallo S A, Finnegan C M, Viard M, et al.: The HIV Env-mediated fusion reaction. Biochim Biophys Acta 2003; 1614: 36-50.
25 Poignard P, Saphire E O, Parren P W, and Burton D R: gp120: Biologic aspects of structural features. Annu Rev Immunol 2001; 19: 253-274.
26 Wyatt R, and Sodroski J: The HIV-1 envelope glycoproteins: fusogens, antigens, and immunogens. Science 1998; 280: 1884-1888.
27 D'Souza M P, Livnat D, Bradac J A, and Bridges S H: Evaluation of monoclonal antibodies to human immunodeficiency virus type 1 primary isolates by neutralization assays: performance criteria for selecting candidate antibodies for clinical trials. AIDS Clinical Trials Group Antibody Selection Working Group. J Infect Dis 1997; 175: 1056-1062.
28 Burton D R, Pyati J, Koduri R, et al.: Efficient neutralization of primary isolates of HIV-1 by a recombinant human monoclonal antibody. Science 1994; 266: 1024-1027.
29 Trkola A, Pomales A B, Yuan H, et al.: Cross-clade neutralization of primary isolates of human immunodeficiency virus type 1 by human monoclonal antibodies and tetrameric CD4-IgG. J Virol 1995; 69: 6609-6617.
30 Trkola A, Purtscher M, Muster T, et al.: Human monoclonal antibody 2G12 defines a distinctive neutralization epitope on the gp120 glycoprotein of human immunodeficiency virus type 1. J Virol 1996; 70: 1100-1108.
31 Muster T, Steindl F, Purtscher M, et al.: A conserved neutralizing epitope on gp41 of human immunodeficiency virus type 1. J Virol 1993; 67: 6642-6647.
32 Mascola J R, Snyder S W, Weislow O S, et al.: Immunization with envelope subunit vaccine products elicits neutralizing antibodies against laboratory-adapted but not primary isolates of human immunodeficiency virus type 1. J Infect Dis 1996; 173: 340-348.
33 Parren P W, Moore J P, Burton D R, and Sattentau Q J: The neutralizing antibody response to HIV-1: viral evasion and escape from humoral immunity. AIDS 1999; 13 Suppl A: S137-S162.
34 Graham B S, and Mascola J R: Lessons from failure—preparing for future HIV-1 vaccine efficacy trials. J Infect Dis 2005; 191: 647-649.
35 Gilbert P B, Peterson M L, Follmann D, et al.: Correlation between immunologic responses to a recombinant glycoprotein 120 vaccine and incidence of HIV-1 infection in a phase 3 HIV-1 preventive vaccine trial. J Infect Dis 2005; 191: 666-677.
36 Flynn N M, Forthal D N, Harro C D, et al.: Placebo-controlled phase 3 trial of a recombinant glycoprotein 120 vaccine to prevent HIV-1 infection. J Infect Dis 2005; 191: 654-665.
37 Burton D R, Desrosiers R C, Doms R W, et al.: HIV vaccine design and the neutralizing antibody problem. Nat Immunol 2004; 5: 233-236.
38 Center R J, Lebowitz J, Leapman R D, and Moss B: Promoting trimerization of soluble human immunodeficiency virus type 1 (HIV-1) Env through the use of HIV-1/simian immunodeficiency virus chimeras. J Virol 2004; 78: 2265-2276.
39 Si Z, Phan N, Kiprilov E, and Sodroski J: Effects of HIV type 1 envelope glycoprotein proteolytic processing on antigenicity. AIDS Res Hum Retroviruses 2003; 19: 217-226.
40 Schulke N, Vesanen M S, Sanders R W, et al.: Oligomeric and conformational properties of a proteolytically mature, disulfide-stabilized human immunodeficiency virus type 1 gp140 envelope glycoprotein. J Virol 2002; 76: 7760-7776.
41 Herrera C, Klasse P J, Michael E, et al.: The impact of envelope glycoprotein cleavage on the antigenicity, infectivity, and neutralization sensitivity of Env-pseudotyped human immunodeficiency virus type 1 particles. Virology 2005; 338: 154-172.
42 Pancera M, and Wyatt R: Selective recognition of oligomeric HIV-1 primary isolate envelope glycoproteins by potently neutralizing ligands requires efficient precursor cleavage. Virology 2005; 332: 145-156.
43 Binley J M, Sanders R W, Master A, et al.: Enhancing the proteolytic maturation of human immunodeficiency virus type 1 envelope glycoproteins. J Virol 2002; 76: 2606-2616.
44 Sanders R W, Vesanen M, Schuelke N, et al.: Stabilization of the soluble, cleaved, trimeric form of the envelope glycoprotein complex of human immunodeficiency virus type 1. J Virol 2002; 76: 8875-8889.
45 Beddows S, Schulke N, Kirschner M, et al.: Evaluating the Immunogenicity of a Disulfide-Stabilized, Cleaved, Trimeric Form of the Envelope Glycoprotein Complex of Human Immunodeficiency Virus Type 1. J. Virol. 2005; 79: 8812-8827.
46 Long E M, Martin H L, Jr, Kreiss J K, et al.: Gender differences in HIV-1 diversity at time of infection. Nat Med 2000; 6: 71-75.
47 Poss M, and Overbaugh J: Variants from the diverse virus population identified at seroconversion of a clade A human immunodeficiency virus type 1-infected woman have distinct biological properties. J Virol 1999; 73: 5255-5264.
48 Roben P, Moore J P, Thali M, et al.: Recognition properties of a panel of human recombinant Fab fragments to the CD4 binding site of gp120 that show differing abilities to neutralize human immunodeficiency virus type 1. J Virol 1994; 68: 4821-4828.
49 Zwick M B, Labrijn A F, Wang M, et al.: Broadly neutralizing antibodies targeted to the membrane-proximal external region of human immunodeficiency virus type 1 glycoprotein gp41. J Virol 2001; 75: 10892-10905.
50 Thali M, Moore J P, Furman C, et al.: Characterization of conserved human immunodeficiency virus type 1 gp120 neutralization epitopes exposed upon gp120-CD4 binding. J Virol 1993; 67: 3978-3988.
51 Wyatt R, Moore J, Accola M, et al.: Involvement of the V1/V2 variable loop structure in the exposure of human immunodeficiency virus type 1 gp120 epitopes induced by receptor binding. J Virol 1995; 69: 5723-5733.
52 Thali M, Furman C, Ho D D, et al.: Discontinuous, conserved neutralization epitopes overlapping the CD4-binding region of human immunodeficiency virus type 1 gp120 envelope glycoprotein. J Virol 1992; 66: 5635-5641.
53 Mariani R, Kirchhoff F, Greenough T C, et al.: High frequency of defective nef alleles in a long-term survivor with nonprogressive human immunodeficiency virus type 1 infection. J Virol 1996; 70: 7752-7764.
54 Parren P W, Wang M, Trkola A, et al.: Antibody neutralization-resistant primary isolates of human immunodeficiency virus type 1. J Virol 1998; 72: 10270-10274.
55 Trkola A, Ketas T J, Nagashima K A, et al.: Potent, broad-spectrum inhibition of human immunodeficiency virus type 1 by the CCR5 monoclonal antibody PRO 140. J Virol 2001; 75: 579-588.
56 Strizki J M, Xu S, Wagner N E, et al.: SCH-C (SCH 351125), and orally bioavailable, small molecule antagonist of the chemokine receptor CCR5, is a potent inhibitor of HIV-1 infection in vitro and in vivo. Proc Natl Acad Sci USA 2001; 98: 12718-12723.
57 Marozsan A J, Kuhmann S E, Morgan T, et al.: Generation and properties of a human immunodeficiency type 1 isolate resistant to the small molecule CCR5 inhibitor, SCH-417690 (SCH-D). Virology 2005; 338: 182-199.
58 Trkola A, Dragic T, Arthos J, et al.: CD4-dependent, antibody-sensitive interactions between HIV-1 and its co-receptor CCR-5. Nature 1996; 384: 184-187.
59 Fouts T, Godfrey K, Bobb K, et al.: Crosslinked HIV-1 envelope-CD4 receptor complexes elicit broadly cross-reactive neutralizing antibodies in rhesus macaques. Proc Natl Acad Sci USA 2002; 99: 11842-11847.
60 Herrera C, Spenlehauer C, Fung M S, et al.: Nonneutralizing antibodies to the CD4-binding site on the gp120 subunit of human immunodeficiency virus type 1 do not interfere with the activity of a neutralizing antibody against the same site. J Virol 2003; 77: 1084-1091.
61 Si Z, Cayabyab M, and Sodroski J: Envelope glycoprotein determinants of neutralization resistance in a simian-human immunodeficiency virus (SHIV-HXBc2P 3.2) derived by passage in monkeys. J Virol 2001; 75: 4208-4218.
62 Zwick M B, Kelleher R, Jensen R, et al.: A novel human antibody against human immunodeficiency virus type 1 gp120 is V1, V2, and V3 loop dependent and helps delimit the epitope of the broadly neutralizing antibody immunoglobulin G1 b12. J Virol 2003; 77: 6965-6978.
63 Moore J P, Cao Y, Leu J, et al.: Inter- and intraclade neutralization of human immunodeficiency virus type 1: genetic clades do not correspond to neutralization serotypes but partially correspond to gp120 antigenic serotypes. J Virol 1996; 70: 427-444.
64 Moore J P, and Sodroski J: Antibody cross-competition analysis of the human immunodeficiency virus type 1 gp120 exterior envelope glycoprotein. J Virol 1996; 70: 1863-1872.
65 Gordon C J, Muesing M A, Proudfoot A E, et al.: Enhancement of human immunodeficiency virus type 1 infection by the CC-chemokine RANTES is independent of the mechanism of virus-cell fusion. J Virol 1999; 73: 684-694.
66 Yuan W, Craig S, Yang X, and Sodroski J: Inter-subunit disulfide bonds in soluble HIV-1 envelope glycoprotein trimers. Virology 2005; 332: 369-383.
67 Owens R J, and Compans R W: The human immunodeficiency virus type 1 envelope protein precursor acquires aberrant intermolecular disulfide bonds that may prevent normal proteolytic processing. Virology 1990; 179: 827-833.
68 Binley J M, Wrin T, Korber B, et al.: Comprehensive cross-clade neutralization analysis of a panel of anti-human immunodeficiency virus type 1 monoclonal antibodies. J Virol 2004; 78: 13232-13252.
69 Moore J P, McCutchan F E, Poon S W, et al.: Exploration of antigenic variation in gp120 from clades A through F of human immunodeficiency virus type 1 by using monoclonal antibodies. J Virol 1994; 68: 8350-8364.
70 Moore J P, Parren P W, and Burton D R: Genetic subtypes, humoral immunity, and human immunodeficiency virus type 1 vaccine development. J Virol 2001; 75: 5721-5729.
71 Hartley O, Klasse P J, Sattentau Q J, and Moore J P: V3: HIV's switch-hitter. AIDS Res Hum Retroviruses 2005; 21: 171-189.
72 Connor R I, Sheridan K E, Lai C, Zhang L, and Ho D D: Characterization of the functional properties of env genes from long-term survivors of human immunodeficiency virus type 1 infection. J Virol 1996; 70: 5306-5311.
73 Zwick M B, Komori H K, Stanfield R L, et al.: The long third complementarity-determining region of the heavy chain is important in the activity of the broadly neutralizing anti-human immunodeficiency virus type 1 antibody 2F5. J Virol 2004; 78: 3155-3161.
74 Murakami T, Ablan S, Freed E O, and Tanaka Y: Regulation of human immunodeficiency virus type 1 Env-mediated membrane fusion by viral protease activity. J Virol 2004; 78: 1026-1031.
75 Wyma D J, Jiang J, Shi J, et al.: Coupling of human immunodeficiency virus type 1 fusion to virion maturation: a novel role of the gp41 cytoplasmic tail. J Virol 2004; 78: 3429-3435.
76 Wyss S, Dimitrov A S, Baribaud F, et al.: Regulation of human immunodeficiency virus type 1 envelope glycoprotein fusion by a membrane-interactive domain in the gp41 cytoplasmic tail. J Virol 2005; 79: 12231-12241.
77 Scanlan C N, Pantophlet R, Wormald M R, et al.: The broadly neutralizing anti-human immunodeficiency virus type 1 antibody 2G12 recognizes a cluster of alpha1→2 mannose residues on the outer face of gp120. J Virol 2002; 76: 7306-7321.
78 Zwick M B, Jensen R, Church S, et al.: Anti-human immunodeficiency virus type 1 (HIV-1) antibodies 2F5 and 4E10 require surprisingly few crucial residues in the membrane-proximal external region of glycoprotein gp41 to neutralize HIV-1. J Virol 2005; 79: 1252-1261.
79 Ofek G, Tang M, Sambor A, et al.: Structure and mechanistic analysis of the anti-human immunodeficiency virus type 1 antibody 2F5 in complex with its gp41 epitope. J Virol 2004; 78: 10724-10737.
80 Moore J P, Cao Y, Qing L, et al.: Primary isolates of human immunodeficiency virus type 1 are relatively resistant to neutralization by monoclonal antibodies to gp120, and their neutralization is not predicted by studies with monomeric gp120. J Virol 1995; 69: 101-109.

Experimental Details II

Introduction

According to the 2005 World Health Organization AIDS epidemic update, there are over 40 million people infected with the HIV virus worldwide, with close to 5 million newly infected cases just last year (1). Among the hardest hit areas is sub-Saharan Africa, with over 25 million people living with HIV and about 10% dying of AIDS-related illnesses. It has been widely recognized and accepted that prophylactic measures in the form of an HIV vaccine, in addition to therapeutic medicines, need to be implemented to curtail the spread of AIDS globally.

An effective HIV vaccine needs to demonstrate an ability to elicit neutralizing antibodies (NAb) that would be capable of blocking the fusogenic interaction and entry of HIV with the CD4 receptor on CD4⁺ helper T cells, mediated by the cell surface viral env glycoproteins, gp120 and gp41. Since the genetic polymorphism of the HIV-1 gag and env genes are diverse and constantly evolving due to rapid mutation within individuals (2), the NAbs targeting the gp120 and gp41 envelope proteins on the viral surface need to be capable of blocking the viral interaction with the CD4 receptor and thereby neutralize viruses from a broad range of subtypes, without discrimination.

One logical design of recombinant env vaccine candidates is to base the vaccine sequence on currently existing HIV-1 isolates that are prevalent in the infected population. To this end, several oligomeric env proteins from several different subtypes or “clades” have been described, with subtype B sequences serving as a basis for the majority of those that have been reported (3-11, 29, 31). The oligomeric env protein complex on the surface of the virus is comprised of a gp120-gp41 heterodimer present in a homotrimer configuration (held together via non-covalent interactions), resembling a “spike” structure. These glycoproteins are derived from a gp160 precursor protein, which undergoes processing and cleavage in the cell to result in gp120 and gp41 heterodimers that are then targeted to the surface of the HIV viral envelope (12, 13). Fusion of the virus with the CD4⁺ cell membrane and oligomerization of the trimer spike is mediated by the gp41 glycoprotein, which is tethered to the virion surface via its transmembrane domain (12, 13).

It has been reasoned that design of a recombinant vaccine should mimic the native trimer spike of the HIV envelope against which NAbs would naturally be generated. Since the native Env trimer is technically challenging to produce in a recombinant form, modified versions of the trimer that could serve as potential vaccine templates have been reported. One typical modification is truncation of the gp41 transmembrane domain from the precursor gp160 to yield gp140 proteins in a soluble form. However, following processing and cleavage, the resulting gp120 and gp41 ectodomain or gp41_{ECTO} (lacking the transmembrane domain) have been shown to form unstable associations and tend to dissociate into their respective monomeric subunits (13, 14).

To address these issues, subtype B HIV_{JR-FL} Env was used as a template and a disulfide bond was introduced between gp120-gp41_{ECTO} subunits (SOS gp140), followed by a further modification to gp41_{ECTO} (I559P mutation), which successfully allowed for the expression of stable, cleaved and fully processed oligomeric gp140 proteins in a trimeric conformation (SOSIP gp140) (8-11, 15-17, and WO 2003/022869). While immunization of rabbits performed with the engineered HIV-1_{JR-FL} SOSIP gp140 elicited antibodies capable of neutralization, the activity was limited primarily to the homologous strain, with only a modest and limited ability to neutralize across different HIV-1 primary isolates (11).

While the SOSIP technology addresses stability and expression, another issue that has limited production and purification of the recombinant trimers has been the spontaneous association of the oligomeric gp140 proteins into aberrant “aggregate” species (3, 9, 11, 18). These aggregate species, typically identified by their reduced mobility on blue native PAGE (BN-PAGE) and non-reduced SDS-PAGE have been difficult to purify from the SOSIP gp140 trimer without compromising yield and/or stability of the trimer. Attempts to fully characterize the aggregate have been limited and their true nature remains elusive.

To explore a wider variety of oligomeric env proteins that could elicit higher breadths of cross-neutralization activity and serve as potential vaccine immunogens, a panel of subtype A sequences from HIV-1 primary isolates in sub-Saharan Africa were studied (19). The env proteins from these sequences were expressed as SOSIP gp140 proteins, with a further engineered mutation at the gp120-gp41_{ECTO} cleavage site (R6) for enhanced furin cleavage (>95% efficiency) to yield soluble, stable and fully processed gp140 trimers. Described herein is the purification and biochemical characterization of KNH1144 SOSIP R6 gp140, derived from a contemporary East African subtype A HIV-1 primary isolate, using methodologies that improve on currently implemented purification procedures. The purified KNH1144 SOSIP R6 gp140 is a trimer based on BN-PAGE and size exclusion chromatography (SEC). In addition, described herein are novel findings of the effects of non-ionic detergents such as Tween 20 on the KNH1144 SOSIP R6 aggregates (19). These findings reveal new insights into the nature of the aggregate species. The effects of non-ionic detergent, e.g., Tween® 20, treatment on the antigenic properties of KNH1144 SOSIP R6 gp140 aggregates and trimers were examined. Finally, digital imaging based on negative stain electron microscopy was performed and revealed the structure of purified KNH1144 SOSIP R6 gp140 as trimeric oligomers.

Materials and Methods

Subtype A KNH1144 SOSIP R6 Transfection and Expression:

The KNH 1144 SOSIP R6 envelope and furin DNA plasmids were as described. For a typical 8 L preparation, HEK 293T cells were seeded in triple flasks at a density of 2.5×10⁷ cells/flask and cultured in DMEM/10% FBS/1% pen-strep with 1% L-glutamine 24 hours prior to transfection. On the day of transfection, 270 ug of KNH1144 SOSIP R6 envelope DNA was mixed with 90 ug of Furin protease DNA plasmid (per flask) in Opti-MEM. Polyethyleneimine (PEI) was added stepwise (2 mg PEI: 1 mg total DNA) and vortexed immediately in between each addition. The PEI/DNA complex solutions were incubated for 20 minutes at room temperature. Complexes were then added to the flasks and incubated for 6 hours at 32° C., 5% CO₂. The cells were then washed with warmed PBS and then incubated in exchange media (DMEM/0.05% BSA/1% pen-strep) for 48 hours at 32° C., 5% CO₂. After the 48 hour incubation, the supernatants were collected and a cocktail of protease inhibitors was added to minimize protein degradation. Harvested supematants were then clarified by filtration through a 0.45 um filter and concentrated to 53×. Expression of KNH1144 gp120 monomer has been previously described (1) and typically, 1-2 L of cell culture supernatants from transfected cells were harvested. Supernatants were clarified by filtration and stored at −80° C. without any concentration prior to purification.

Purification of KNH1144 SOSIP R6 gp140 and gp120:

KNH1144 SOSIP R6 gp140 trimer was purified via a four step process starting with an ammonium sulfate precipitation followed by lectin affinity, size exclusion and ion-exchange chromatography. 53× concentrated cell culture supernatant was precipitated with an equal volume of 3.8 M ammonium sulfate to remove contaminant proteins (with the major contaminant being α-2-macroglobulin). The ammonium sulfate was added with constant stirring with a stir bar and then was immediately centrifuged at 4000 rpm, 4° C. for 45 minutes. The resulting supernatant was diluted 4-fold with PBS, pH 7.25, and was filtered using a 0.45 um vacuum filter. The sample was then loaded at 0.5-0.8 ml/min onto a Galanthus nivalis (GNA) lectin (Vector Laboratories, Burlingame, Calif.) column equilibrated with PBS-pH 7.25. Once the load was finished, the column was washed with PBS pH 7.25 until OD₂₈₀ reached baseline, followed by a second wash with 0.5 M NaCl PBS pH 7.25 at 1 ml/min in order to remove contaminant proteins (mainly BSA). The column was then eluted with 1 M MMP PBS pH 7.25 starting with flowing one half CV through the column at 0.3 ml/min and pausing the purification for a 1 hour incubation in MMP elution buffer. Following the incubation, the flow was restarted at 0.3 ml/min and 0.5-1 ml fractions were collected. All peak fractions were then pooled and concentrated to a final volume of 1 ml using a Vivaspin 100,000 MWCO concentrator (Vivascience, Edgewood, N.Y.) centrifuged at 1000×g. The concentrated lectin elution was applied over a Superdex 200 SEC column (GE Healthcare, Piscataway, N.J.) equilibrated in 20 mM Tris pH 8, 200 mM NaCl (TN-200), injecting 0.5 ml of sample per run and was resolved at 0.4 ml/min, collecting 0.4 ml fractions. The fractions were analyzed by BN-PAGE using a 4-12% Bis-Tris NuPAGE gel (Invitrogen, Carlsbad, Calif.) (10). All trimer containing fractions were pooled and diluted to 75 mM NaCl with 20 mM Tris pH 8. The diluted SEC pool was then applied over a 1 ml HiTrap DEAE FF column (GE Healthcare), equilibrated in 20 mM Tris pH 8, 75 mM NaCl (TN-75). The diluted SEC pool was loaded at 0.5 ml/min. The column was washed with TN-75 at 1 ml/min until the OD₂₈₀ reached baseline. The column was then eluted with 20 mM Tris, 300 mM NaCl pH 8 at 1 ml/min, collecting 0.5 ml fractions.

To maximize trimer yield, the flow-through fraction from the DEAE column was re-applied over the column (equilibrated in TN-75) and typically 20-30% or 30-40% more trimer was recovered in this manner. The fractions were analyzed by BN-PAGE and by reducing and non-reducing SDS-PAGE. Western blot analysis on non-reduced SDS-PAGE gel was performed with the ARP3119 monoclonal antibody. The trimer containing fractions were pooled and trimer concentration was determined through densitometry on a reducing SDS-PAGE gel using JR-FL gp120 as a standard.

KNH1144 gp120 Monomer:

Unconcentrated cell culture supernatants containing secreted gp120 monomer were applied directly over a GNA lectin column equilibrated in 20 mM imidazole pH 7.1 at 1-2 ml/min. Following adsorption, the column was washed with a high salt (PBS containing 1 M NaCl, pH 7.1) wash, followed by a low salt (20 mM imidazole pH 7.1) wash. The column was eluted with 1 M MMP in 20 mM imidazole, 0.2 M NaCl pH 7.1. Peak fractions were pooled and diluted with 20 mM imidazole, pH 7.1, thirteen-fold to a final buffer concentration of 20 mM imidazole, pH 7.1, 15 mM NaCl. The diluted GNA elution was applied over 1 ml HiTrap Q Sepharose FF (GE Healthcare) equilibrated in 20 mM imidazole, pH 7.1. Following binding, the column was washed with 20 mM imidazole, pH 7.1, and was eluted with 20 mM imidazole, 0.2 M NaCl, pH 7.1. The Q elutions were pooled and concentrated and applied over a Superdex 200 column equilibrated in PBS in 0.5 ml volumes and resolved at 0.4 ml/min. Peak fractions were analyzed by 4-12% Bis-tris gels (Invitrogen), followed by Coomassie staining. Fractions containing gp120 were pooled and quantified as described above for the SOSIP R6 gp140 trimers and stored at −80° C.

Tween® 20 Aggregate “Conversion”/“Collapse” Experiments:

Tween® 20 Dose effect: 1 ug of purified KNH1144 SOSIP R6 trimer was incubated with varying concentrations of Tween® 20 (polyoxyethylene sorbitan monolaurate) ranging from 0 to 0.0001% (v/v) and incubated for 1 hour at room temperature. Following incubation, samples were analyzed by BN-PAGE as described above.

Kinetics of Tween® 20 effect: To ascertain the early kinetics of the Tween® 20 effect on aggregate, 1 ug of purified KNH1144 SOSIP R6 trimer was incubated with Tween® 20 at a final concentration of 0.05% (v/v) for 5 minutes and for 10 minutes. A no-detergent control was included separately for each timepoint.

Temperature dependance on Tween® 20 effect: To determine if temperature affected the ability of Tween® 20 to recover trimers from aggregates (i.e., collapse aggregate into trimer), 1 ug of purified KNH1144 SOSIP R6 trimer was incubated with Tween® 20 to a final concentration of 0.05% (v/v) at 0° C. (on ice), room temperature (22-23° C.) at 37° C., or left untreated for 10 minutes. Following the incubation, samples were analyzed by BN-PAGE and Coomassie staining.

Tween® 20 effect on KNH1144 gp120: To test if Tween® 20 had a similar effect on KNH1144 gp120, 1 ug of purified gp120 monomer was either untreated or incubated with Tween® 20 at a final concentration of 0.05% for 10 minutes at room temperature. Following the treatment, samples were analyzed by BN-PAGE and Coomassie staining.

Tween® 20 effect on α-2-macroglogulin (α₂M): 0.5 ug of purified α-2-macroglobulin was either untreated or treated with Tween® 20 at a final concentration of 0.05% for 10 minutes at room temperature. Reactions were analyzed via BN-PAGE, followed by Coomassie staining.

Size Exclusion Chromatography (SEC) Analysis:

All runs were performed at 4° C. on the AKTA FPLC system (GE Healthcare). Each run was performed at least twice.

Molecular weight standards SEC: A Superdex 200 10/300 GL column was equilibrated in 20 mM Tris pH 8, 0.5 M NaCl (TN-500) and calibrated with the following molecular weight standard proteins: thyroglobulin 669,000 Da; ferritin 440,000 Da; BSA 67,000 Da; and RNAse A 13,700 Da. A standard curve was generated by plotting the observed retention volumes of the standard proteins against the log values of their predicted molecular weights.

KNH1144 gp120 SEC analysis: 14 ug of purified KNH1144 gp120 (either untreated or Tween® 20-treated as described above) was applied over the Superdex 200 column equilibrated in TN-500 and resolved at a flow rate of 0.4 ml/min. As a control, 10-14 ug of JR-FL gp120 was also analyzed in a similar manner.

KNH1144 SOSIP R6 gp140 SEC analysis: 8-10 ug of purified KNH1144 SOSIP R6 gp140 was treated with Tween® 20 at a final concentration of 0.05% for 10-30 minutes at room temperature. Treated samples were then applied over the Superdex 200 column equilibrated with TN-500 containing 0.05% Tween® 20 (TNT-500) and resolved at 0.4 ml/min, collecting 0.4 ml fractions. Trimer-containing fractions were then analyzed by BN-PAGE, followed by silver staining. Fractions were also separated by BN-PAGE, followed by Western blot analysis with ARP 3119 antibody.

Blue Native PAGE (BN-PAGE) and SDS-PAGE Analysis:

All SDS-PAGE analysis (reduced and non-reduced) were performed using 4-12% Bis-Tris NuPage gels (Invitrogen). BN-PAGE analysis was performed as described (10). Silver stain analysis was performed with the SilverQuest kit (Invitrogen). Coomassie G-250 stain was performed using either the SimplyBlue SafeStain or Easy-to-Use Coomassie® G-250 Stain (Invitrogen).

Antigenicity Experiments—Lectin ELISA:

Human mAbs b6 (32), b12 (33) and 2G12 (26), HIVIg (40) were obtained from Dr. Dennis Burton (The Scripps Research Institute, La Jolla, Calif.) or Dr. Herman Katinger (University of Natural Resources and Applied Life Sciences, Austria, Vienna). For the lectin based ELISA, anti-Env antibodies 2G12, b6, b12 and HIVIg were used. In addition, the CD4-IgG2 antibody conjugate PRO 542 (39) was also used.

ELISA plates were coated overnight at 4° C. with lentil lectin powder from Lens culinaris (L9267, Sigma) at 10 ug/ml concentration. Plates were washed with PBS twice and blocked with SuperBlock (Pierce) (warmed to RT). Excess blocking agent was washed off with PBS. SEC fractions containing HMW aggregate were either untreated or treated with 0.05% Tween® 20 (v/v, final concentration) for 30 minutes at room temperature (RT) and were added at 0.3 ug/ml (diluted in PBS) and bound to the plates (via the lectin) for 4 hours at RT. Following binding, plates were washed 4 times with PBS and incubated with primary anti-Env antibodies starting at 10 ug/ml in PBS/5% milk. 4× serial dilutions were performed and incubations were performed for 3 hours at RT. Following antibody incubation, plates were washed 6 times and goat anti-human IgG (H+L) alkaline phosphatase conjugate secondary antibody (Jackson ImmunoResearch) was added at 1/4000 concentration in PBS/5% milk. Plates were washed 4 times and ELISAs were developed using the Ampak detection system (Dako Cytomation, Carpinteria, Calif.) as per the manufacturer's instructions.

DEAE Anion Exchange Chromatography of Tween® 20-Treated KNH1144 SOSIP R6 gp140 Trimers:

Purified KNH1144 SOSIP R6 gp140 trimers, treated either with or without 0.05% Tween® 20 (final), containing a₂M contaminant in TN-75 buffer was applied over 1 ml DEAE HiTrap FF column (equilibrated in TN-75) at 0.25 ml/min at RT and flow-through (FT) fractions were collected. Following sample loading, the column was washed with TN-75 at 0.5 ml/min and wash fractions were collected. Finally, the column was eluted with TN-300 and equal amounts from each fraction were analyzed via BN-PAGE, followed by Coomassie G-250 staining.

Electron Microscopy:

EM analysis of the SOSIP trimers was performed by negative stain as previously described (34, 35). Because this technique is incompatible with detergent, 20 μl of the original sample (0.5 mg/ml in TN-300) was dialyzed against BSB (0.1 M H₃BO₃, 0.025 M Na₂B₄O₇, 0.075 M NaCl, pH 8.3) and subsequently depleted of detergent using the Mini Detergent-OUT™ detergent removal kit (Calbiochem, La Jolla, Calif.) as described by the manufacturer. Two microliters of the resulting protein solution, diluted in 200 μl BSB, was affixed to carbon support membrane, stained with 1% uranyl formate, and mounted on 600 mesh copper grids for analysis. EMs were recorded at ×100,000 at 100 kV on a JOEL JEM 1200 electron microscope. Measurements were made using the Image-Pro Plus software program. Fifty or more trimers were measured and analyzed statistically. The average diameter of the compact trimers formed by the SOSIP gp140 (e.g., KNH1144.R6 SOSIP) proteins was about 12-13 nm.

Results

Expression and Purification of Trimeric KNH1144 SOSIP R6 gp140:

The purification of KNH1144 SOSIP R6 gp140 trimers typically involved three chromatography steps: GNA lectin affinity, Superdex 200 size exclusion and DEAE weak anion exchange. 53× concentrated cell culture supernatant precipitated with ammonium sulfate was clarified by centrifugation, diluted and applied over the GNA lectin affinity column to capture gp140 proteins via (α-1, 3) mannose residues. Analysis of the ammonium sulfate precipitation using different starting concentrations of harvested cell culture supernatant (100× to 40×) revealed that 53× was the optimum condition at which maximum α-2-macroglobulin precipitated out, with minimal envelope protein loss. While the GNA lectin column was highly efficient in capture of the gp140 trimer, elution of the protein under even extremely mild conditions, with the competing MMP eluant, caused significant de-stabilization of the trimer and resulted in marked dissociation of the trimer into dimer and monomer species. Attempts to separate the different oligomeric gp140 species via Superdex SEC resulted in efficient separation of the monomer from the dimer and trimer. Superdex 200 SEC of the GNA eluate yielded trimers that were free of monomers, but not of dimers. To resolve trimers away from dimers (and residually co-migrating monomers), a DEAE anion exchange step was incorporated, which led to very efficient separation of dimer from trimer, thereby yielding pure trimers at the end of the purification protocol.

SDS-PAGE analysis under reducing conditions showed that the final preparation was of high purity (at least 90%), with only the gp120 moeity visible on the reduced gel (FIG. 9, left panel, center lane). Common serum contaminants that were detectable by reducing SDS-PAGE were α-2-macroglobulin (a₂M) and BSA, which typically comprised up to ˜10% of the final preparation. The non-reduced gel shows intact gp140 protein on SDS-PAGE (FIG. 9, left panel, right lane). In addition, little to no disulfide-linked aggregate (typically revealed as migrating much slower on a non-reducing gel) was detected. This was confirmed by anti-envelope Western blot analysis on the non-reduced gel (FIG. 9, Anti-Env blot, middle panel). BN-PAGE analysis of the purified trimer revealed the purified trimer to migrate between the 669 k thyroglobulin and 440 k ferritin marker proteins (FIG. 9, right panel, SOSIP R6). This is consistent with the migration patterns for JR-FL SOSIP gp140 which has been observed to migrate in the lower range of 669 k and 440 kDa (9, 10, 11). An additional slower migrating band, typically classified as high molecular weight (HMW) SOSIP aggregates and comprising about 30% of the preparation, was also detected (FIG. 9, right panel, SOSIP R6, −lane). Typical HMW aggregate content ranged from 10 to 40% of the final preparation prior to non-ionic detergent treatment. Treatment of the purified preparation with Tween® 20 at a final concentration of 0.05% converted the HMW aggregate species to trimers, yielding a homogenous trimer preparation (FIG. 9, right panel, SOSIP R6, +lane)(19). It should be noted that treatment with Tween® 20 also caused the treated trimer to migrate slightly more rapidly than the untreated trimer (notice faster mobility of trimer in the +lane).

Purification of the monomeric protein yielded a homogenous preparation as evident by a single band when analyzed by reducing SDS-PAGE (FIG. 9, left panel, left lane) and Superdex 200 SEC. BN-PAGE analysis of the purified monomer, either in the presence or absence of Tween® 20 revealed a single migrating monomeric gp120 species, devoid of any higher order oligomers, consistent with its purity on SDS-PAGE (FIG. 9, right panel, gp120−/+lanes).

Since Tween® 20 provided a simple and mild means to obtain homogenous trimers, further characterization of the non-ionic detergent effect was performed. A purified trimer preparation containing ˜30% aggregates (e.g., monomer, dimmer and trimer) was treated with Tween® 20 at final concentrations of 0.0001% to 0.1% (v/v) (FIG. 10A). The SOSIP R6 aggregates were converted to trimers at concentrations of 0.1% to 0.01% (FIG. 10A, lanes 3-5). No conversion was observed at Tween® 20 concentrations of 0.001 and 0.0001% (FIG. 10A, lanes 6 and 7). Close examination of the 0.01% reaction (lane 5) revealed that traces of aggregate were present, thus indicating that 0.01% Tween® 20 is probably the threshold concentration. To study the kinetics of the conversion, trimer preparations containing ˜30% aggregate were incubated with Tween® 20 for 0, 5 and 10 minutes prior to analysis by BN-PAGE. As shown in FIG. 10B, both the 5 minute and 10 minute incubations completely eliminated the aggregate, indicating that the kinetics of the reaction was rapid and within a 5 minute time span.

The effect of temperature on aggregate rearrangement was also examined. Aggregate/trimer preparations were incubated with Tween® 20 either at 0° C. (on ice), room temperature (22-23° C.), or 37° C. As shown in FIG. 10C, conversion of aggregate to trimer occurred at all 3 temperatures, indicating that the Tween® 20 effect on aggregate was independent of temperature over this range. Similar results were obtained when Tween® 80 was used instead of Tween® 20.

Similar Tween® 20 treatment of the gp120 monomer showed that there was no difference observed in its migratory pattern either in the presence or absence of Tween® 20, indicating that Tween® 20 did not affect the gp120 monomer (FIG. 9, right panel, gp120, −/+lanes). In some cases, a mild increase in the staining intensity of the gp120 monomer occurred.

To test if the detergent had a collapsive effect on another large multi-subunit protein, α-2-macroglobulin (α₂M), which is an acidic 726 kDa tetrameric glycoprotein comprised of four identical 185 kDa subunits, was incubated with Tween® 20. No change was observed in the migratory pattern of α₂M in the presence of Tween® 20, although there was a slight increase in the staining intensity of the protein. (See FIGS. 7 and 8)

To examine whether Tween® 20 could convert preparations containing predominantly aggregate as the major oligomeric species to resulting trimers, a KNH1144 SOSIP R6 preparation containing >70% HMW aggregate was incubated with Tween® 20 and analyzed by BN-PAGE. As shown in FIG. 10D, Tween® 20 was effective in converting the aggregate rich fraction to trimer (FIG. 10D, left panel). Fractions of less purity containing HMW aggregate, dimers and monomers (FIG. 10D, right panel, −lane, each species denoted by arrows), when treated with Tween® 20 also resulted in collapse of HMW aggregate to resulting trimer (FIG. 10D, right panel, +lane). However, no effect on dimer or monomer migration was observed (FIG. 10D, right panel, +lane, arrows), indicating that the Tween® 20 action was specific to KNH1144 SOSIP R6 HMW aggregate and timer. Consistent with previous observations, some increase in monomer staining was observed. Thus, these results indicate that Tween® 20 efficiently converts the KNH1144 SOSIP HMW aggregate into trimeric form. According to this invention, Tween® 20 efficiently converted into trimers MAW preparations having greater than 10%, (e.g., greater than 10-40%), aggregate. Greater than 90-99%, or 100%, trimers were able to be recovered from non-ionic detergent-, e.g., Tween® 20, treated HMW aggregates.

SEC Analysis of KNH1144 gp120 Monomer and SOSIP R6 gp140 Trimer:

Size exclusion chromatography (SEC) analysis was performed as a second means to characterize the molecular sizes of KNH1144 gp120 monomer and SOSIP R6 gp140 trimer proteins. A Superdex 200 size exclusion column was calibrated with thyroglobulin (669 kDa), ferritin (440 kDa), BSA (67 kDa) and RNAse A (13.7 kDa) as molecular weight standards. In addition, monomeric JR-FL gp120 was also analyzed as a control. KNH1144 gp120 and JR-FL gp120 were each found to migrate at an apparent molecular weight of 210 kDa (see FIGS. 7 and 8). These values are consistent with those found for JR-FL gp120 (10).

To further study the oligomeric nature of the KNH1144 SOSIP R6 gp140 trimer, final purified preparations were treated with Tween® 20 prior to analysis on Superdex 200 SEC to yield homogenous and unambiguous trimer samples devoid of HMW aggregate. Initial studies showed re-formation of HMW aggregate when treated trimer samples were resolved in non-detergent TN-500 buffer on the SEC column. The resulting mixed trimer-aggregate fractions, presumably re-formed upon separation of the Tween® 20 from the gp140 oligomers in non-detergent buffer, was considered unsuitable for SEC analysis due to its heterogeneous nature.

In order to maintain homogenous trimers, treated trimer was resolved in the presence of TN-500 containing 0.05% Tween® 20 (TNT-500). As shown in FIG. 11, (bottom panel BN-PAGE), the trimer (thick arrow) migrated from fractions B10 through C2, represented in the major peak, with its peak signal at fraction B12 (vertical arrow). The retention time at this fraction corresponds to an apparent calculated molecular weight of ˜518 kDa. The reported apparent molecular weight (MW) of JR-FL SOSIP gp140 trimer calculated via Superdex 200 SEC analysis is ˜520 kDa (9); and thus, the calculated apparent MW value for KNH1144 SOSIP R6 gp140 trimer is consistent with MW values of other SOSIP envelope trimers.

Effect of Tween® 20 Treatment on KNH1144 SOSIP R6 Antigenicity:

Studies of the antigenic properties of unpurified KNH1144 SOSIP R6 gp140 (19) showed that it was immunoprecipitated by the neutralizing molecules 2G12, b12, CD4-IgG2, as well as the non-neutralizing mAb b6. The experiments described herein further assessed the effect of the Tween® 20 aggregate collapse on the antigenic properties of KNH1144 SOSIP HMW aggregates to determine if conversion of HMW aggregate into trimer favorably enhanced antigenicity.

SEC fractions containing ≧80% KNH1144 SOSIP R6 HMV aggregate content (as shown in FIG. 10D, −lane) were either untreated or Tween® 20 treated (typical reaction is represented in FIG. 10D). The antigenicity of the proteins in the presence and absence of Tween® 20 was examined using a lectin based ELISA. These results are shown in FIG. 12A. All the anti-env antibodies and CD4-IgG2, displayed increased binding to the Tween® 20 treated aggregate. The above experiments were performed on Tween® 20 converted trimer, using preps containing >80% HMW aggregate.

To demonstrate that Tween® 20 treatment did not unfavorably disrupt the above antibody epitopes on trimers, similar lectin ELISAs were performed using 2G12, b6, b12 and CD4-IgG2 on SOSIP R6 gp140 trimers that contained low amounts of HMW aggregate (˜10-15% content) that were either untreated or treated with Tween® 20. As shown in FIG. 12B, no significant differences were observed in the antigenicity of trimer in presence or absence of Tween® 20. Unfortunately, since the HMW aggregate species is present in very limiting quantities, the Tween® 20 effect was assessed using only the above mentioned mAbs. These results show that Tween® 20 treatment and consequential conversion of HMW aggregate to resulting trimer enhances epitope exposure for Env binding antibodies. Thus Tween® 20 treatment and presence may offer favorable consequences in the context of KNH1144 SOSIP R6 gp140 trimer stability and antibody epitope exposure.

Effect of Tween® 20 Treatment on the Ionic Properties of KNH1144 SOSIP R6 gp140 Trimer:

DEAE anion exchange chromatography was used to examine the effect of Tween® 20 on the ionic properties of SOSIP R6 gp140 and control proteins. Untreated or Tween® 20 treated KNH1144 SOSIP R6 gp140 trimer spiked with a₂M contaminating protein (which is unaffected by Tween® 20 and binds to anion exchange resins) were applied over DEAE anion exchange column (FIG. 13, Load). The column was washed and eluted and fractions were analyzed via BN-PAGE and Coomassie staining and is shown in FIG. 13. As expected, untreated SOSIP R6 gp140 trimer and the a₂M contaminant bound to the DEAE column and were recovered in the elution fraction (FIG. 13, Untreated control, top panel, denoted by asterisks). However, upon treatment with Tween® 20, the KNH1144 SOSIP R6 gp140 trimer was found in the flow-through (FT) fractions of the column (FIG. 13, Tween® 20 treated, bottom panel, FT, denoted by asterisks), indicating that it did not bind to the DEAE, unlike the untreated trimer. Residual trimer is further recovered in the wash fraction (FIG. 13, Wash). In contrast, the a₂M contaminant, which was used as the internal control, bound to the DEAE column and was recovered in the elution, indicating that it was unaffected by the presence of Tween® 20 (FIG. 9, Tween® 20 treated, bottom panel, Elution).

In other similar experiments, in which BSA, another acidic protein was substituted as the contaminant, similar results were obtained. This indicates that Tween® 20 treatment may exert its action on KNH1144 SOSIP R6 HMW aggregate and timer through a combination of hydrophobic interactions that possibly involve perturbations in inter- and/or intra-subunit charge-charge interactions, as examined by DEAE anion exchange chromatography.

Electron Microscopy and Digital Imaging of KNH1144 SOSIP R6 gp140 Trimers:

Electron microscopy was performed on purified SOSIP R6 preparations employing negative stain EM analysis. The results, shown in FIG. 10, reveal that the majority of the observed structures displayed a regular compact morphology with approximate three-fold symmetry. This tri-lobed configuration is most apparent in preparations with deeper stain (FIG. 10; panel of trimers) that are less subject to the flattening that can occur in thinner staining preparations.

Initially, for the EM studies, it was found that the uranyl formate negative straining technique was not compatible with detergent-containing buffers. However, some trimeric structures of the anticipated dimensions were observed in the poorly staining preparations. Thereafter, the KNH1144 SOSIP preparation was subjected to a detergent removal protocol, which yielded improved staining. Following detergent removal, the majority of the observed structures displayed a regular compact morphology with approximate three-fold symmetry (e.g., FIG. 10). This configuration is most apparent in preparations with deeper stain that are less subject to the flattening that can occur in thinner staining preparations.

In order to calculate diameters of the trimers, 70 spikes in the shallow stain samples were scored and a diameter of 13.5±1.73 nm was calculated. Seventy eight (78) trimers from the deep stain were scored and resulted in a diameter of 11.6 nm±1.75 nm. The shallow stain preparation likely gives a slight overestimation of the size and the deep stain preparation gives a slightly underestimated size. Therefore, the true size is likely to be 12.6±1.74 nm (i.e., and in line with authentic Env spikes measured in situ on both negatively stained, as well as unstained, cryo-EM preparations of SW (36, 37). Thus the biophysical EM analysis of KNH1144 SOSIP R6 gp140 is in good agreement with the above biochemical data and confirms the oligomeric status of the purified KNH1144 env complex as being trimeric.

Discussion

In the context of identifying and pursuing a variety of HIV-1 Env-based protein vaccines, described herein is the purification and characterizion of a novel subtype A KNH1144 trimeric envelope spike protein and its properties. Several novel insights were gained as a result of these studies, which revealed the biochemical effects of Tween® 20 on the oligomeric conformations of the KNH1144 SOSIP R6 proteins. Until the present invention, only one subtype B envelope, HIV-1 JR-FL has been manipulated to a purified form to mimic as closely as possible the native trimeric structure of the HIV-1 viral surface envelope complex via the SOSIP technology (8-11, 15-17). The present invention provides another clade, clade A KNH1144, for which the SOSIP technology results in purified trimeric envelopes that are stable, soluble, and fully cleaved.

The purification process implemented according to the present invention for the KNH1144 SOSIP trimers provides a marked improvement over that utilized for JR-FL SOSIP gp140 trimers. For the KNH1144 SOSIP, the GNA lectin column provided a significant enrichment of gp140 proteins, but elution off the column significantly destabilized the gp140 trimers, resulting in a compromise of timer fidelity on the column. As a result, significant dissociation of the trimer to resulting dimer and monomer was noticed. This destabilization could be brought about from Galanthus Nivalis lectin binding to α1-3 and α1-6 mannose linkages on the gp140 high mannose chains, which are internal linkages and not terminal linkages (20). During elution, the affinity of the lectin for the mannan is likely much higher than the intersubunit protein-protein affinities of the 3 gp120-gp41_{ECTO} monomers contributing to trimer formation, resulting in destabilization and dissociation into component dimers and monomers. To alleviate some measure of the destabilization that could be caused due to resulting sheer stresses during elution, a one hour incubation in MMP eluting buffer was included. So while a highly enriching step, the lectin affinity column also decreased the final yield of trimer significantly, due to its dissociation during the elution phase.

The next step in the purification, Superdex 200 SEC, while somewhat efficient in resolving away monomer, was not very effective in resolution of dimer from trimer. The incorporation of a DEAE weak anion exchange chromatography step was very efficient in resolving dimer (and residual monomer) away from trimer, resulting in trimeric KNH1144 SOSIP R6 gp140 of high purity. Notably, binding (and retention) of the trimer occurred under a relatively polar environment (vis-à-vis ion exchange) at 75 mM NaCl, while dimer and monomer flowed through the DEAE column under these conditions.

It is relevant to extrapolate from its behavior on anion exchange chromatography that the nature of the KNH1144 SOSIP R6 g140 trimer is that of an acidic protein, which would be contrary to its predicted basic isoelectric point (pI) of 8.73 calculated for the protein backbone. However, the likely presence of the predicted acidic sialylated complex oligosaccharide chains on the gp140 (21, 22) would contribute to a decrease in the overall charge of the glycoprotein and thus confer on it properties of an acidic protein. Indeed, analysis of purified KNH1144 SOSIP R6 gp140 trimers on isoelectric focusing gels reveal it to migrate at a pI range of 5.9 to 6.1, consistent with the above observations.

The purified trimer was shown to contain variable amounts of HMW aggregate (FIG. 9, right panel, EN-PAGE), which could not be attributed to being formed at any one particular step of the purification, although one possibility might be at the lectin elution step. As mentioned before, one of the key improvements made in this purification protocol is absence of SDS-insoluble aggregates in the final prep, which are formed by abberantly formed disulfide bonds and are visualized by their slow migration on a non-reduced SDS-PAGE. As detected by Coomassie staining and confirmed by anti-envelope Western blot, little to no SDS-insoluble aggregates were observed (FIG. 9, left and middle panels, Non-Red SDS-PAGE and Anti-Env blot). This is in contrast to what was observed with JR-FL SOSIP gp140 (R6 and non-R6 versions), where SDS-insoluble aggregates comprised a significant percentage of the final preparations (9, 10, 11).

Based on observations regarding non-ionic detergent treatments of KNH1144 SOSIP R6 gp140 trimers (19), Tween® 20 was used to address the co-purifying HMW aggregate present in the final trimer preparations. Tween® 20 was chosen because initial observations had shown that Tween® 20 treatment was mild and did not result in any detectable monomer formation, unlike treatment with the other non-ionic detergents NP-40 and Triton X-100, where dimers and monomers were observed upon treatment (19). Tween® 20 treatment of the final purified KNH1144 SOSIP R6 trimer preparation was highly reproducible and resulted in the “conversion” of the HMW aggregate species, as shown in FIG. 9 (right panel, BN-PAGE). Since this resulted in a single, homogenous, oligomeric species of KNH1144 SOSIP R6 gp140 trimers, we routinely incorporated it as the final step in our preparations. Further analysis using reduced SDS-PAGE gels showed that the purified trimer was fully cleaved, with practically undetectable uncleaved protein (as visualized by both Coomassie staining and Western blot analysis) (FIG. 9, left panel, Red SDS-PAGE). The initial purifications were performed using a non-R6 version of KNH1144 SOSIP gp140, which resulted in ˜40-50% of uncleaved protein in the final preparation, prompting the development of the R6 version. This also represents another improvement over JR-FL SOSIP R6 gp140 trimers, where cleavage of gp120-gp41_{ECTO} was not as efficient (9, 11).

In order to expand the initial Tween® 20 observations to the stability of HMW aggregates, a variety of experiments were performed to characterize the effect of Tween® 20 and to better understand its mechanism of action. As shown in FIG. 6, the effect of Tween® 20 is dose dependent, time dependent and temperature independent within the parameters that were examined. Its effect is remarkably specific to KNH1144 SOSIP R6 HMW aggregate and trimers and has no effect on gp120 monomers, or KNH1144 SOSIP R6 dimers. In addition, other similar large, macromolecular, acidic proteins such as a₂M are not affected by the detergent. Initially, the hypothesis was that the Tween® 20 specifically interacted with points of gp120-gp41_{ECTO} intersubunit contact within the HMW aggregate, presumably in a hydrophobic manner. In this context, the HMW aggregate would have to be comprised of some multiple of trimer (most likely a dimer of trimers), since detergent treatment specifically results in a “rearrangement” to a trimeric configuration. The specificity of this reaction can further be defined by the observation that dimeric KNH1144 SOSIP R6 gp140 proteins are unaffected and do not undergo the collapse (FIG. 6D). In addition, Tween® 20 treatment would also seem to cause the trimer to assume a more compact configuration, as evident by its slightly more rapid mobility on BN-PAGE (FIG. 9).

While the anti-flocculatory effects of non-ionic detergents on aggregates of macromolecular proteins such as antibodies (immunoglobulins, for example) are well known and documented, the mechanisms of their actions have been realized to be largely by pre-emption of unfavorable hydrophobic interactions by detergent intercalation. Tween® 20, however, would seem to exert its action in a somewhat paradoxical mechanism, since treatment of the KNH1144 SOSIP R6 gp140 trimer with the detergent renders it unable to interact with anion exchange resins such as DEAE (FIG. 9, bottom panel, Tween® 20 treated), indicating that the overall charge of the trimer was being affected by the detergent.

Since the nature of non-ionic detergents is exactly that, i.e., non-ionic, it is difficult to realize how an uncharged molecule such as Tween® 20 would affect the charge status of a large, macromolecular oligomer such as the KNH1144 SOSIP R6 trimer. Furthermore, this effect is highly specific to the trimer, as other such large, highly charged (acidic) oligomeric proteins such as a₂M and even smaller ones such as BSA are unaffected by the detergent. One hypothesis that has emerged from this invention is that perhaps the Tween® 20 was “coating” the trimer in a manner that may cause perturbations in its conformation, resulting in its “compactness”. These perturbations would be of a subtle nature which involve the various points of contact between the individual component gp140 monomers, causing disruption and destabilization of interactions that favor the HMW aggregate conformation. A consequence of these perturbations would be “shielding” of ionic charges that would normally be exposed (and contribute to binding to ion exchange resins). It is reasonable to speculate that perhaps the charges that are “shielded” are those on the sialic acid residues of the complex carbohydrate chains, since these would be most likely to be highly exposed at the surface (21, 22). Tween® 20 and Tween® 80 are polyoxyethylene sorbitan esters of fatty acids and thus may likely interact with the sialic acids, causing a charge “neutralization” effect. The involvement of the sialic acid residues can be investigated by mild sialidase treatment (21, 22) and removal of these residues, followed by Tween® 20 treatment, followed by monitoring of binding on ion exchange resins.

To further biochemically characterize the purified KNH1144 monomeric and trimeric envelope proteins, size exclusion chromatography analyses were performed in order to ascertain their apparent molecular masses. These were performed on Tween® 20 treated trimers that were devoid of any HMW aggregates and thus consisted of only one homogeneously oligomeric species, i.e., the trimer, and therefore would yield unambiguous results. The retention times of the KNH1144 SOSIP R6 gp140 trimer resulted in a calculated apparent molecular weight of ˜518 kDa. This is consistent with the reported calculated apparent molecular weight of 520 kDa for the other SOSIP gp140 trimer, JR-FL SOSIP gp140 (9). The predicted molecular weight for a trimer such as KNH1144 (and JR-FL) would be ˜420 kDa (3×140 kDa monomers). Thus, similar to JR-FL SOSIP gp140, the KNH1144 SOSIP R6 gp140 trimer also exhibits an abberant migration on SEC, presumably due to interactions of its N-linked glycans with the dextran—(agarose polymer) based matrix of Superdex 200, resulting in a higher than expected apparent molecular mass. In addition, envelope proteins have been shown to be non-globular in shape (10, 23, 24); therefore, gel filtration may not be optimal for determination of their precise molecular masses. This also extends to the KNH1144 gp120 monomer as well. Values of ˜210 kDa were obtained for KNH1144 gp120 and the control JR-FL gp120 (see FIGS. 11 and 12). The reported value for JR-FL gp120 is 200 kDa (10); accordingly, the obtained values are well within the expected range (given that molecular weight determination via SEC is not extremely accurate, unlike other methodologies such as mass spectrometry). Thus, gp120, whose predicted molecular weight ranges from ˜95 to ˜120 kDa, results in an abberant migratory pattern on SEC, presumably due to its glycan interactions with the sizing column matrix. It should be noted that unlike the KNH1144 SOSIP R6 gp140 trimer, migration of KNH1144 gp120 (and JR-FL gp120) were not affected by the presence or absence of Tween® 20, consistent with the initial BN-PAGE observations (FIG. 9, right panel, gp120).

While it would seem that the presence of Tween® 20 for KNH1144 SOSIP R6 gp140 proteins would be advantageous, possible Tween® 20 effects on the antigenicity of the HMW aggregate and trimer were examined. Effects on antigenicity was examined by performing lectin ELISAs with the NAbs 2G12, b12, HIVIg, the CD4-IgG2 antibody conjugate PRO 542, as well as the non-neutralizing mAb b6, to gain information on neutralizing/non-neutralizing epitope exposure and accessibility. It was reasoned that trimer preparations containing 10-30% HMW aggregate may not undergo significant enough changes that would be detectable in a non-quantitative assay such as IPs, i.e., subtle changes (20-30% changes) may go undetected in such an assay due to sensitivity. However, samples representing extremes may undergo significantly high changes that should be detectable in an assay format such as ELISA. Therefore, SEC fractions that contained ≧80% HMW aggregate were used, which would reflect one extreme prior to Tween® 20 treatment and the resulting trimer, which would reflect the other extreme post treatment. A representative reaction of this is illustrated in FIG. 10D.

As shown in FIG. 12A, significant epitope exposures were observed upon Tween® 20 rearrangement of the HMW aggregate to trimer, and these changes were noticed for all of the anti-env agents. These changes indeed were not as apparent in trimer preparations that were predominantly trimer, with low aggregate content (10-15%) (FIG. 12B). Thus the treated, purified trimer displays antigenic properties similar to that which was previously observed with crude, unpurified trimer supernatants, i.e., binding to 2G12, b6, b12 and PRO 542 (19). In the context of HIVIg, which is a low neutralizing polyclonal human antisera directed against gp120 hypervariable loop (40), it can be inferred that this epitope is accessible on the surface of the HMW aggregate, based on its ability to bind the antibody in absence of Tween® 20. Consistent with the other anti-Env agents examined here, HIVIg epitope exposure also significantly increased on the rearranged trimer, upon treatment with Tween® 20. The likely explanation to these increases in epitope exposure is that “disruption/rearrangement” of the aggregate and its subsequent conversion to trimer unshields the above mentioned surfaces and thus, upon conversion, these surfaces are now exposed on their individual trimers and are accessible to the antibodies. From the context of a single HMW aggregate which is likely to be a multimer of trimers, only a small portion of these epitopes are accessible, most probably due to steric hindrance from adjacently “clumped” SOSIP R6 trimers/oligomers. When the single HMW aggregate is then Tween® 20 converted to resulting trimers, antibody epitopes are now exposed on every one of the resulting individual component trimers, resulting in an increase in antibody accessibility and binding. Thus Tween® 20 treatment and its conversion of the aggregate to trimer do not seem to have detrimental effects on antigenicity and may be favorable to the structural properties of the KNH1144 SOSIP R6 gp140 proteins.

Analysis of KNH1144 SOSIP R6 gp140 proteins by negative stain EM further confirmed the biochemical observations that these gp140 proteins were indeed trimeric in nature (FIG. 14). A distinguishing feature of the KNH1144 SOSIP R6 construct, in comparison to other similar constructs of trimerized gp120 and gp140, is its compact nature. Most other constructs show either predominantly loosely associated subunits or a mix of loosely and tightly associated subunits (5, 18, 38). The observation that the KNH1144 SOSIP R6 trimer is compact is associated with anti-Env antibody epitope availability. EM on Tween®-treated trimer which has favorable anti-Env epitope exposure was performed. It is somewhat incongruous from a purely steric standpoint that a “compact” trimer would also have improved epitope exposure, a consequence expected from a “loose” or “elongated” structure. Immunoelectron microscopy analyses with the above mentioned antibodies will further address the exposure of epitopes on trimeric forms.

The present invention expands the panel of trimeric HIV-1 envelope proteins that may be used as protein-based HIV-1 vaccine candidates or serve as a template for future design of Env based protein vaccine candidates, using the SOSIP technology. Immunological studies in rabbits with JR-FL SOSIP R6 gp140 trimers, while effective in eliciting NAbs, were limited in their breadth of neutralization of primary HIV-1 isolates (11). Factors associated with the biochemical nature of the JR-FL SOSIP gp140 and other oligomeric Env proteins that are thought to limit their observed immunological response in animals, such as inefficient furin cleavage of the gp120-gp41_{ECTO} cleavage site giving rise to heterogenous trimers (containing both cleaved and uncleaved trimers), presence of SDS-insoluble aggregates and presence of undesirable gp140 oligomers such as dimers and monomers (5, 6, 9, 10, 11, 27-30) have been issues needing resolution.

The description of the KNH1144 SOSIP R6 gp140 trimers of the present invention addresses most of these issues. Furthermore, the description of the Tween® 20 affects on coverting HMW aggregates to trimeric forms further expands on current knowledge of the aggregate species in HIV-1 biology. Of significance, it was shown for the first time, that oligomeric Env protein complexes designed using the SOSIP technology platform are indeed trimeric from EM images and that the trimers are of a similar diameter as native spikes on the HIV-1 virion (36, 37). Expansion of the panel of potential HIV-1 SOSIP protein vaccine candidates by development of a clade A envelope according to this invention now allows for immunological evaluation of the KNH1144 SOSIP R6 gp140 trimer in small animals, for example. Such evaluations will assist in determining the efficacy of KNH1144 SOSIP R6 gp140 trimers as immunogens capable of eliciting broadly neutralizing immune responses directed against HIV-1.

### REFERENCES

1. UNAIDS: AIDS Epidemic Update: December 2005, ISBN 92 9 173439 X.
2. Kijak, G. H., and McCutchan, F. E. (2005) Curr Infect Dis Rep. 7, 480-488.
3. Jeffs, S. A., Goriup, S., Kebble, B., Crane, D., Bolgiano, B., Sattentau, Q., Jones, S., and Holmes, H. (2004) Vaccine. 22, 1032-1046.
4. Srivastava, I. K., Stamatatos, L., Legg, H., Kan, E., Fong, A., Coates, S. R., Leung, L., Wininger, M., Donnelly, J. J., Ulmer, J. B., and Barnett, S. W. (2002) J Virol. 76, 2835-2847.
5. Srivastava, I. K., Stamatatos, L., Kan, E., Vajdy, M., Lian, Y., Hilt, S., Martin, L., Vita, C., Zhu, P., Roux, K. H., Vojtech, L., Montefiori, D. C., Donnelly, J., Ulmer, J. B., and Barnett, S. W. (2003) J Virol. 77, 11244-11259.
6. Yang, X., Florin, L., Farzan, M., Kolchinsky, P., Kwong, P. D., Sodroski, J., and Wyatt, R. (2000) J Virol. 74, 4746-4754.
7. Grundner, C., Li, Y., Louder, M., Mascola, J., Yang, X., Sodroski, J., and Wyatt R. (2005) Virology 331, 33-46.
8. Binley, J. M.; Sanders, R.. W., Clas, B., Schuelke, N., Master, A., Guo, Y., Kajumo, F., Anselma, D. J., Maddon, P. J., Olson, W. C., and Moore, J. P. (2000) J Virol. 74, 627-643.
9. Sanders, R. W., Vesanen, M., Schuelke, N., Master, A., Schiffner, L., Kalyanaraman,

R., Berkhout, B., Maddon, P. J., Olson, W. C., Lu, M., and Moore, J. P. (2002) J Virol. 76, 8875-8889.
10. Schulke, N., Vesanen, M. S., Sanders, R. W., Zhu, P., Lu, M., Anselma, D. J., Villa, A. R., Parren, P. W., Binley, J. M., Roux, K. H., Maddon, P. J., Moore, J. P., and Olson, W. C. (2002) J Virol. 76, 7760-7776.
11. Beddows, S., Schulke, N., Kirschner, M., Barnes, K., Franti, M., Michael, E., Ketas, T., Sanders, R. W., Maddon, P. J., Olson, W. C., and Moore, J. P. (2005) J Virol. 79, 8812-8827.
12. Emini, E. A. and Koff, W. C. (2004) Science 304, 1913-1914.
13. Poignard, P., Saphire, E. O., Parren, P. W., and Burton, D. R. (2001) Annu Rev Immunol. 19, 253-274.
14. Wyatt, R., and Sodroski, J. (1998) Science 280, 1884-1888.
15. Sanders, R. W., Schiffner, L., Master, A., Kajumo, F., Guo, Y., Dragic, T., Moore, J. P., and Binley, J. M. (2000) J Virol. 74, 5091-5100.
16. Binley, J. M., Sanders, R. W., Master, A., Cayanan, C. S., Wiley, C. L., Schiffner, L., Travis, B., Kuhmann, S., Burton, D. R., Hu, S. L., Olson, W. C., and Moore, J. P. (2002) J Virol. 76, 2606-2616.
17. Binley, J. M., Cayanan, C. S., Wiley, C., Schulke, N., Olson, W. C., and Burton, D. R. (2003) J Virol. 77, 5678-5684.
18. Pancera, M., Lebowitz, J., Schon, A., Zhu, P., Freire, E., Kwong, P. D., Roux, K. H., Sodroski, J., and Wyatt, R. (2005) J Virol. 79, 9954-9969.
19. Beddows, S., Kirschner, M., Campbell-Gardener, L., Franti, M., Dey, A, K., Iyer, S, N., Paluch, M., Master, A., Overbaugh, J., VanCott, T., Olson, W. C., and Moore, J. P. (2006) AIDS Res Hum Retroviruses (in press).
20. Botos, I., and Wiodawer, A. (2005) Prog Biophys Mol Biol. 88, 233-282.
21. Scanlan, C. N., Pantophlet, R., Wormald, M. R., Ollmann, Saphire, E., Stanfield, R., Wilson, I. A., Katinger, H., Dwek, R. A., Rudd, P. M., and Burton, D. R. (2002) J Virol. 76, 7306-7321.
22. Sanders, R. W., Venturi, M., Schiffner, L., Kalyanaraman, R., Katinger, H., Lloyd, K. O., Kwong, P. D., and Moore, J. P. (2002) J Virol. 76, 7293-7305.
23. Center, R. J., Earl, P. L., Lebowitz, J., Schuck, P., and Moss, B. (2000) J Virol. 74, 4448-4455.
24. Center, R. J., Schuck, P., Leapman, R. D., Arthur, L. O., Earl, P. L., Moss, B., and Lebowitz, J. (2001) Proc Natl Acad Sci USA. 98, 14877-14882.
25. Trkola, A., Pomales, A. B., Yuan, H., Korber, B., Maddon, P. J., Allaway, G. P., Katinger, H., Barbas, C. F. 3rd, Burton, D. R., Ho, D. D., and Moore, J. P. (1995) J Virol. 69, 6609-6617.
26. Trkola, A., Purtscher, M., Muster, T., Ballaun, C., Buchacher, A., Sullivan, N., Srinivasan, K., Sodroski, J., Moore, J. P., and Katinger, H. (1996) J Virol. 70, 1100-1108.
27. Center, R. J., Lebowitz, J., Leapman, R. D., and Moss, B. (2004) J Virol. 78, 2265-2276.
28. Chakrabarti, B. K., Kong, W. P., Wu, B. Y., Yang, Z. Y., Friborg, J., Ling, X., King, S. R., Montefiori, D. C., and Nabel, G. J. (2002) J Virol. 76, 5357-5368.
29. Zhang, C. W., Chishti, Y., Hussey, R. E., and Reinherz, E. L. (2001) J Biol Chem. 276, 39577-39585.
30. Yang, X., Lee, J., Mahony, E. M., Kwong, P. D., Wyatt, R., and Sodroski, J. (2002) J Virol. 76, 4634-4642.
31. Lian, Y., Srivastava, 1., Gomez-Roman, V. R., Zur Megede, J., Sun, Y., Kan, E., Hilt, S., Engelbrecht, S., Himathongkham, S., Luciw, P. A., Otten, G., Ulmer, J. B., Donnelly, J. J., Rabussay, D., Montefiori, D., van Rensburg, E. J., and Barnett, S. W. (2005) J Virol. 79, 13338-13349.
32. Roben, P., Moore, J. P., Thali, M., Sodroski, J., Barbas, C. F. 3rd., and Burton, D. R. (1994) J Virol. 68, 4821-4828.
33. Burton, D. R., Pyati, J., Koduri, R., Sharp, S. J., Thornton, G. B., Parren, P. W. H. I., Sawyer, L. S. W., Hendry, R. M., Dunlop, N., Nara, P. L., Lamacchia, M., Garratty, G., Stiehm, E. R., Bryson, Y. J., Cao, Y., Moore, J. P., Ho, D. D., and Barbas, C. F. 3rd. (1994) Science 266, 1024-1027.
34. Roux, K. H. (1989) Methods Enzymol. 178, 130-144.
35. Roux, K. H. (1996) Methods 10, 247-256.
36. Zhu, P., Chertova, E., Bess, J., Jr., Lifson, J. D., Arthur, L. O., Liu, J., Taylor, K. A., and Roux, K. H. (2003) Proc Natl Acad Sci USA. 100, 15812-15817.
37. Qiao, Z. S., Kim, M., Reinhold, B., Montefiori, D., Wang, J. H., and Reinherz, E. L. (2005) J Biol Chem. 280, 23138-23146.
38. Olson, W. C., and Maddon, P. J. (2003) Curr Drug Targets Infect Disord. 3, 255-262.

Experimental Details III

Materials and Methods

Subtype B 5768.4 SOSIP R6 gp140 Expression and Purification:

The subtype B 5768.4 envelope sequence has been described (8). The sequence was modified to make the soluble SOSIP R6 gp140 version, as described above for the KNH1144 isolate in the section “Experimental Details I” and for the JR-FL SOSIP R6 gp140 trimers (3, 4). DNA synthesis was performed by DNA 2.0 (Menlo Park, Calif.). The 5768.4 SOSIP R6 gp140 trimer was expressed in HEK293 and small scale (2 L) purification was performed as described above for KNH1144 SOSIP R6 gp140.

Detergent Aggregate “Collapse”/“Conversion” Experiments:

Tween® 20 Dose effect: 1 ug of purified 5768.4 SOSIP R6 trimer was incubated with varying concentrations of Tween® 20 ranging from 0.1 to 0.0001% (v/v) and incubated for 1 hour at room temperature. Following incubation, samples were analyzed by BN-PAGE as described above. Detergent effect on 5768.4 SOSIP R6 gp140 trimer preparations: 0.24 ug of purified 5768.4 SOSIP R6 trimer was incubated with Triton X-100, NP-40, or SDS to a final detergent concentration of 0.1% (v/v) or with Tween® 20 at concentrations of 0.05 and 0.1% for 1 hour at room temperature. Following incubation, 4× BN-PAGE MOPS sample buffer was added and the samples were immediately analyzed on BN-PAGE at 150 V for 2 hours at room temperature, followed by Coomassie G-250 staining.

Size Exclusion Chromatography (SEC) Analysis:

All runs were performed at 4° C. on the AKTA FPLC system (GE Healthcare). Each run was performed at least twice.

Molecular weight standards SEC: Superdex 200 10/300 GL column was equilibrated in 20 mM Tris pH 8, 0.5 M NaCl (TN-500) and calibrated with the following molecular weight standard proteins: thyroglobulin 669,000 Da; ferritin 440,000 Da; BSA 67,000 Da; RNAse A 13,700 Da. A standard curve was generated by plotting the observed retention volumes of the standard proteins against the log values of their predicted molecular weights.

Blue Native PAGE (BN-PAGE), SDS-PAGE and Western Blot Analysis:

All SDS-PAGE analysis (reduced and non-reduced) were performed using 4-12% Bis-Tris NuPage gels (Invitrogen). BN-PAGE analysis was performed as described before (1-3). Silver staining analysis was performed with the SilverQuest kit (Invitrogen).

Results and Discussion

Detergent “Collapse” Effect on Subtype B 5768.4 HMW Aggregate:

Detergent treatments were performed on a trimeric gp140 of a different subtype 5768.4, which is a subtype B envelope (8). The 5768.4 Env protein was modified to the SOSIP R6 version, expressed and purified as a gp140 trimer. The purified final preparation is shown in FIG. 15 (BN-PAGE, untreated lane) and contains high HMW aggregate content (˜60%) and minor a₂M contamination (˜5%), with trimer comprising the rest. Purified preparations were incubated with the various indicated detergents (Triton X-100, NP40, SDS, or Tween® 20) and were treated to collapse HWM aggregate.

As shown in FIG. 15, Tween® (Tween® 20 and Tween® 80) effectively collapsed HMW aggregate to trimers at 0.1 and 0.05% concentrations. Triton X-100 was also capable of collapsing HMW aggregate to trimer, however, some breakdown to monomeric 5768.4 was observed. As expected, SDS was effective in breaking down the entire 5768.4 gp140 protein to resulting monomers by virtue of its denaturing effect on the trimer and HMW aggregate. NP40 treatment also led to collapse of HMW aggregate to trimer, but the resulting trimer displayed a somewhat broader staining compared with that of Tween® 20 treated trimers. Thus, the detergent effect, in particular, Tween® 20, on the HMW aggregate is not unique to KNH1144 SOSIP gp140 Env proteins and exhibits similar HMW aggregate collapse ability on other subtypes of HIV envelope trimers as well, such as the 5768.4 SOSIP R6 gp140.

### REFERENCES FOR EXPERIMENTAL DETAILS III

1. Schulke et. al, 2002, Journal of Virology 76: 7760-7776.
2. Binley et. al, 2002, Journal of Virology 76: 2606-2616.
3. Beddows et. al, 2005, Journal of Virology 79: 8812-8827.
4. Sanders et. al, 2002, Journal of Virology 76: 8875-8889.
5. Grunder et. al, 2005, Virology 331: 33-46.
6. Pancera et. al, 2005, Journal of Virology 79: 9954-9969.
7. Trkola et. al, 1996, Nature 384: 184-187.
8. Li et. al, 2005, Journal of Virology 79: 10108-10125.

Experimental Details IV

This Example describes a rabbit immunogenicity study that was performed using both purified KNH1144 SOSIP Env trimers and KNH1144 gp120 Env monomers as immunogens. The design of this study is shown in FIG. 16. The direct comparison of the immunogenicity between the SOSIP trimer and gp120 monomer forms of KNH1144 Env was conducted with a protein-only dosing regimen. Arms I to IV in this study utilized Quil A as adjuvant, while arms V and VI utilized Ribi as adjuvant. Arms V and VI received an initial prime with 100 ug, followed by 30 ug boosts. Arms I and II vs. Arms III and IV allowed a direct assessment of the effect of Tween 20® in the immunogen preparations. Four animals (rabbits) were used per group.

KNH1144 SOSIP Vs. gp120:

Rabbits were immunized with env proteins as described for FIG. 16, and sera were evaluated at week 30 for neutralization of env-pseudotyped HIV-1_{KNH1144} at Monogram Biosciences using methods as described in Binley, J. M. et al., 2004. Comprehensive cross-clade neutralization analysis of a panel of anti-human immunodeficiency virus type 1 monoclonal antibodies. J. Virology. 78:13232-13252. Treatment groups included KNH1144 SOSIP or gp120 under the following conditions: Quil A plus Tween 20®, Quil A minus Tween 20®, and Ribi plus Tween 20®. The results, shown in FIG. 17, represent the reciprocal of the dilution that resulted in 50% neutralization of env-pseudotyped HIV-1 on U87.CD4.CCR5 cells. Lines indicate the means and standard deviations for each group. A neutralization titer of 10 (1:10 dilution of sera) represents the lower limit of detection of the assay. Seroconverters represent animals that developed a significant neutralizing response.

TABLE 1
Neutralization of env-pseudotyped HIV-1 by gp120 and SOSIP antisera generated in the rabbit immunogenicity study.

| | IC50 (/1diln) | | | | | | | | | | | | |
|---|---|---|---|---|---|---|---|---|---|---|---|---|---|
| | 93IN905 | 98CN006 | ARW92020 | AUG93077 | MBC8 | KNH1144 | MBP_A22 | MBP_A30 | MN | SF162 | JRCSF | NL43 | aMLV |
| gp120 | <10 | <10 | <10 | <10 | 11 | <10 | <10 | <10 | <10 | 12 | <10 | <10 | <10 |
| (+Tween)/ | | | | | | | | | | | | | |
| Ribi | | | | | | | | | | | | | |
| gp120 | <10 | <10 | <10 | <10 | <10 | <10 | <10 | <10 | <10 | <10 | <10 | <10 | <10 |
| (+Tween)/ | | | | | | | | | | | | | |
| Ribi | | | | | | | | | | | | | |
| gp120 | <10 | <10 | <10 | <10 | <10 | <10 | <10 | <10 | <10 | <10 | <10 | <10 | <10 |
| (+Tween)/ | | | | | | | | | | | | | |
| Ribi | | | | | | | | | | | | | |
| gp120 | <10 | <10 | <10 | <10 | <10 | <10 | <10 | <10 | <10 | <10 | <10 | <10 | <10 |
| (+Tween)/ | | | | | | | | | | | | | |
| Ribi | | | | | | | | | | | | | |
| SOSIP | <10 | <10 | <10 | <10 | [symbol] | <10 | <10 | <10 | [symbol] | [symbol] | <10 | [symbol] | <10 |
| Trimer | | | | | | | | | | | | | |
| (+Tween)/ | | | | | | | | | | | | | |
| Ribi | | | | | | | | | | | | | |
| SOSIP | <10 | <10 | <10 | <10 | [symbol] | <10 | <10 | <10 | [symbol] | [symbol] | <10 | [symbol] | <10 |
| Trimer | | | | | | | | | | | | | |
| (+Tween)/ | | | | | | | | | | | | | |
| Ribi | | | | | | | | | | | | | |
| SOSIP | 11 | <10 | <10 | 13 | [symbol] | <10 | <10 | <10 | [symbol] | [symbol] | <10 | [symbol] | <10 |
| Trimer | | | | | | | | | | | | | |
| (+Tween)/ | | | | | | | | | | | | | |
| Ribi | | | | | | | | | | | | | |
| SOSIP | <10 | <10 | <10 | <10 | [symbol] | [symbol] | <10 | <10 | [symbol] | [symbol] | <10 | [symbol] | <10 |
| Trimer | | | | | | | | | | | | | |
| (+Tween)/ | | | | | | | | | | | | | |
| Ribi | | | | | | | | | | | | | |
| gp120 | <10 | <10 | <10 | <10 | <10 | <10 | <10 | <10 | <10 | <10 | <10 | <10 | <10 |
| (+Tween)/ | | | | | | | | | | | | | |
| Quil A | | | | | | | | | | | | | |
| gp120 | <10 | <10 | <10 | <10 | [symbol] | <10 | <10 | <10 | [symbol] | [symbol] | <10 | [symbol] | <10 |
| (+Tween)/ | | | | | | | | | | | | | |
| Quil A | | | | | | | | | | | | | |
| gp120 | <10 | <10 | <10 | 11 | [symbol] | <10 | <10 | <10 | 22 | [symbol] | <10 | [symbol] | <10 |
| (+Tween)/ | | | | | | | | | | | | | |
| Quil A | | | | | | | | | | | | | |
| gp120 | <10 | <10 | <10 | <10 | 17 | <10 | <10 | <10 | <10 | [symbol] | <10 | <10 | <10 |
| (+Tween)/ | | | | | | | | | | | | | |
| Quil A | | | | | | | | | | | | | |
| SOSIP | <10 | <10 | <10 | 10 | [symbol] | <10 | <10 | <10 | 15 | 12 | <10 | 16 | <10 |
| Trimer | | | | | | | | | | | | | |
| (+Tween)/ | | | | | | | | | | | | | |
| Quil A | | | | | | | | | | | | | |
| SOSIP | <10 | <10 | <10 | <10 | <10 | <10 | <10 | <10 | [symbol] | [symbol] | <10 | [symbol] | <10 |
| Trimer | | | | | | | | | | | | | |
| (+Tween)/ | | | | | | | | | | | | | |
| Quil A | | | | | | | | | | | | | |
| SOSIP | <10 | <10 | <10 | <10 | <10 | [symbol] | <10 | <10 | [symbol] | [symbol] | <10 | <10 | <10 |
| Trimer | | | | | | | | | | | | | |
| (+Tween)/ | | | | | | | | | | | | | |
| Quil A | | | | | | | | | | | | | |
| SOSIP | <10 | <10 | <10 | <10 | <10 | <10 | <10 | <10 | <10 | [symbol] | <10 | <10 | <10 |
| Trimer | | | | | | | | | | | | | |
| (+Tween)/ | | | | | | | | | | | | | |
| Quil A | | | | | | | | | | | | | |
| gp120 | <10 | <10 | <10 | <10 | [symbol] | <10 | <10 | <10 | [symbol] | [symbol] | <10 | [symbol] | <10 |
| (−Tween)/ | | | | | | | | | | | | | |
| Quil A | | | | | | | | | | | | | |
| gp120 | <10 | <10 | <10 | <10 | <10 | <10 | <10 | <10 | <10 | [symbol] | <10 | <10 | <10 |
| (−Tween)/ | | | | | | | | | | | | | |
| Quil A | | | | | | | | | | | | | |
| gp120 | <10 | <10 | <10 | <10 | [symbol] | <10 | <10 | <10 | <10 | [symbol] | <10 | <10 | <10 |
| (−Tween)/ | | | | | | | | | | | | | |
| Quil A | | | | | | | | | | | | | |
| SOSIP | <10 | <10 | <10 | <10 | <10 | <10 | <10 | <10 | <10 | [symbol] | <10 | <10 | <10 |
| Trimer | | | | | | | | | | | | | |
| (−Tween)/ | | | | | | | | | | | | | |
| Quil A | | | | | | | | | | | | | |
| SOSIP | <10 | <10 | <10 | <10 | <10 | <10 | <10 | <10 | <10 | [symbol] | <10 | <10 | <10 |
| Trimer | | | | | | | | | | | | | |
| (−Tween) | | | | | | | | | | | | | |
| Quil A | | | | | | | | | | | | | |
| SOSIP | <10 | <10 | <10 | <10 | <10 | [symbol] | <10 | <10 | [symbol] | [symbol] | <10 | <10 | <10 |
| Trimer | | | | | | | | | | | | | |
| (−Tween)/ | | | | | | | | | | | | | |
| Quil A | | | | | | | | | | | | | |
| SOSIP | <10 | <10 | <10 | <10 | <10 | <10 | <10 | <10 | <10 | [symbol] | <10 | <10 | <10 |
| Trimer | | | | | | | | | | | | | |
| (−Tween)/ | | | | | | | | | | | | | |
| Quil A | | | | | | | | | | | | | |
| Z23 mean | [symbol] | [symbol] | [symbol] | [symbol] | [symbol] | [symbol] | [symbol] | [symbol] | [symbol] | [symbol] | [symbol] | [symbol] | |

IC₅₀ values were determined at Monogram Biosciences as previously described (Binley, J. M. et al., 2004. Comprehensive cross-clade neutralization analysis of a panel of anti-human immunodeficiency virus type 1 monoclonal antibodies. J. Virology. 78:13232-13252). Rabbits were immunized with env proteins as illustrated in FIG. 16, and sera were evaluated at week 30 for neutralization of env-pseudotyped HIV-1_{93IN905} (Clade C), HIV-1_{98CN006} (Clade C), HIV-1_{ARW92020} (Clade A), HIV-1_{AUG93077} (Clade A), HIV-1_{MBC8} (Clade C), HIV-1_{KNH1144} (Clade A), HIV-1_{MBP}_A22 (Clade B), HIV-1_{MBP}_A30 (Clade B), HIV-1_{MN} (Clade B), HIV-1_{SF162} (Clade B), HIV-1_{JR-CSF} (Clade B), and HIV-1_{NL43} (Clade B) using methods previously described (3). Treatment groups are: KNH1144 SOSIP or gp120 with Ribi adjuvant plus Tween 20 ®,KNH1144 SOSIP or gp120 with Tween adjuvant plus Tween 20 ®, and KNH1144 SOSIP or gp120 with Tween adjuvant minus Tween 20 ®. IC₅₀ values represent the reciprocal of the dilution that resulted in 50% neutralization of env pseudotyped HIV-1 on U87.CD4.CCR5 cells. IC₅₀ values were also determined for HIV⁺ plasma against each HIV-1 strain. For the HIV⁺ plasma samples, the assay was performed seven times for each of the HIV-1 strains tested. Amphotropic murine leukemia virus (aMLV) was tested as a negative control. Green shading is used to indicate significant HIV-1 neutralization that is both >50% and >3 times that observed for aMLV. Other results are considered non-specific.

Ribi-Adjuvanted KNH1144 SOSIP Vs. gp120:

Rabbits were immunized with env proteins as described above in this Example, and sera were evaluated at week 30 for neutralization of env-pseudotyped HIV-1_{MBC8}, HIV-1_{MN}, HIV-1_{SF162}, and HIV-1_{NL4-3} at Monogram Biosciences using methods as described by Binley, J. M. et al., 2004. Comprehensive cross-clade neutralization analysis of a panel of anti-human immunodeficiency virus type 1 monoclonal antibodies. J. Virology. 78:13232-13252. For this comparison, treatment arms shown are KNH1144 SOSIP and KNH1144 gp120 in Ribi adjuvant. The results, shown in FIG. 18, represent the reciprocal of the dilution that resulted in 50% neutralization of env-pseudotyped HIV-1 on U87.CD4.CCR5 cells. Lines indicate the mean and standard deviations for each group. A neutralization titer of 10 (1:10 dilution of sera) represents the lower limit of detection of the assay. Seroconverters represent animals that developed a significant neutralizing response against the indicated virus.

Quil A Vs. Ribi Adjuvant Used with Tween 20® Treated KNH1144 SOSIP gp140:

Rabbits were immunized with env proteins as described above, and sera were evaluated at week 30 for neutralization of env-pseudotyped HIV-1_{MBC8}, HIV-1_{MN}, HIV-1_{SF162}, and HIV-1_{NL4-3} using methods as described for FIGS. 17 and 18. For this comparison, treatment arms shown are Ribi or Quil A plus Tween 20® KNH1144 SOSIP. The results, shown in FIG. 19, represent the reciprocal of the dilution that resulted in 50% neutralization of env-pseudotyped HIV-1 on U87.CD4.CCR5 cells. Lines indicate the mean and standard deviations for each group. A neutralization titer of 10 (1:10 dilution of sera) represents the lower limit of detection of the assay. Seroconverters represent animals that developed a significant neutralizing response against the indicated virus.

Presence and Absence of Tween 20®:

Rabbits were immunized with KNH1144 SOSIP (FIG. 20) or KNH1144 gp120 (FIG. 20 continued) as described above, and sera were evaluated at week 30 for neutralization of env-pseudotyped HIV-1_{MBC8}, HIV-1_{MN}, HIV-1_{SF162}, and HIV-1_{NL4-3} at Monogram Biosciences using methods previously described. For this comparison, treatment arms shown are A) Quil A±Tween 20® KNH1144 SOSIP and B) Quil A±Tween 20® KNH1144 gp120. The results represent the reciprocal of the dilution that resulted in 50% neutralization of env-pseudotyped HIV-1 on U87.CD4.CCR5 cells. The lines in FIG. 20 indicate the mean and standard deviations for each group. A neutralization titer of 10 (1:10 dilution of sera) represents the lower limit of detection of the assay. Seroconverters represent animals that developed a significant neutralizing response against the indicated virus.

KNH1144 SOSIP or KNH1144 gp120 as Immunogens, ELISA Analysis:

Rabbits were immunized at study weeks 0, 4, 12, 20, 28 with purified KNH1144 monomeric gp120 or KNH1144 trimeric SOSIP Env proteins. The anti-gp120 antibody responses were assayed by ELISA by measuring binding of sera to monomeric gp120 immobilized on plastic via the C-terminal specific antibody D7324 using alkaline phosphatase conjugate and the AMPAK colorimetric detection system. (FIGS. 21A, 21B and 21C). Data are presented in FIGS. 21A-C as midpoint serum binding titer values obtained by interpolation and represent the average mean value obtained from four rabbits per group. The results shown in FIG. 21A were obtained from rabbits of Group I that were immunized with monomeric KNH1144 gp120 in the presence of Tween, and from rabbits of Group II that were immunized with KNH1144 SOSIP in the presence of Tween 20®. For the Group I and II animals, 30 ug of protein were used per injection with Quil A as adjuvant. The results shown in FIG. 21B were obtained from rabbits of Group III that were immunized with monomeric KNH1144 gp120 in the absence of Tween, and from rabbits of Group IV that were immunized with KNH1144 SOSIP in the absence of Tween 20®. For the Group III and IV animals, 30 ug of protein were used per injection with Quil A as adjuvant. The results shown in FIG. 21C were obtained from rabbits of Group V that were immunized with KNH1144 monomeric gp120 in the presence of Tween 20®, and from rabbits of Group VI that were immunized using KNH1144 SOSIP in the presence of Tween 20®. For the Group V and VI animals, 100 ug of protein was used for the first immunization, and 30 ug of protein was used for subsequent immunizations with RIBI as adjuvant.

This study demonstrates that KNH1144 Env SOSIP trimer is superior to monomeric KNH1144 gp120 Env protein in eliciting neutralizing antibody activity against homologous virus in vivo. KNH1144 SOSIP (i.e., KNH1144 Env SOSIP trimer) is also superior to gp120 monomer in the presence of Ribi adjuvant in eliciting antibodies that neutralize heterologous HIV-1 subtype B and C viruses. This study also demonstrates that Ribi adjuvant provided a modest benefit over Quil A adjuvant in terms of the heterologous neutralizing responses obtained using KNH1144 SOSIP as immunogen. Finally this study demonstrates that Tween 20® treatment provided a modest but consistent improvement in the heterologous neutralizing responses obtained with both KNH1144 SOSIP and gp120 monomer used as immunogens.

1.-72. (canceled)

73. A protein comprising:
(A). (a) a modified gp120 envelope polypeptide portion of a gp140 envelope of an HIV-1 KNH1144 isolate, or a quasi-species thereof; and (b) a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:2 and SEQ ID NO:3, respectively, said modified gp120 envelope polypeptide portion comprising a cysteine at amino acid position 511, and said modified gp41 ectodomain polypeptide portion comprising a cysteine at amino acid position 617 and a proline at amino acid position 571; wherein (i) the amino acid positions are numbered by reference to SEQ ID NO:1; (ii) the modified gp120 envelope polypeptide portion further comprises a mutated furin recognition sequence; and (iii) the modified gp120 polypeptide portion and the modified gp41 ectodomain polypeptide portion are bound to one another by a disulfide bond between the cysteine at amino acid position 511 and the cysteine at amino acid position 617; or
(B). (a) a modified gp120 envelope polypeptide portion of a gp140 envelope of an HIV-1 5768.4 isolate, or a quasi-species thereof; and (b) a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519, and said modified gp41 ectodomain polypeptide portion comprising a cysteine at amino acid position 625 and a proline at amino acid position 579; wherein (i) the amino acid positions are numbered by reference to SEQ ID NO:10; (ii) the modified gp120 envelope polypeptide portion further comprises a mutated furin recognition sequence; and (iii) the modified gp120 polypeptide portion and the modified gp41 ectodomain polypeptide portion are bound to one another by a disulfide bond between the cysteine at amino acid position 519 and the cysteine at amino acid position 625.

74. The protein of claim 73, wherein, in (A) the cysteine at position 511 of the modified gp120 envelope polypeptide portion is the result of an A511C mutation, the cysteine at position 617 of the modified gp41 ectodomain polypeptide portion is the result of a T617C mutation and the proline at position 571 is the result of an I571P mutation; and in (B) the cysteine at position 519 of the modified gp120 envelope polypeptide portion is the result of an A519C mutation, the cysteine at position 625 of the modified gp41 ectodomain polypeptide portion is the result of a T625C mutation and the proline at position 579 is the result of an I579P mutation.

75. The protein of claim 73, wherein, in (A), the modified gp120 polypeptide portion comprises the consecutive amino acid sequence as set forth in SEQ ID NO:2 and the modified gp41 ectodomain polypeptide portion comprises the consecutive amino acid sequence as set forth in SEQ ID NO:3; and in (B), the modified gp120 polypeptide portion comprises the consecutive amino acid sequence as set forth in SEQ ID NO:11 and the modified gp41 ectodomain polypeptide portion comprises the consecutive amino acid sequence as set forth in SEQ ID NO:12.

76. The protein of claim 73, wherein, in (A) or (B), the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii).

77. A trimeric envelope glycoprotein complex comprising three monomers, each of which is the protein of claim 73 (A) or (B).

78. A trimeric envelope glycoprotein complex comprising three monomers, each of which is the protein of claim 74 (A) or (B).

79. The trimeric complex of claim 77, further comprising a non-ionic detergent.

80. The trimeric complex of claim 78, further comprising a non-ionic detergent.

81. The trimeric complex of claim 79, wherein the non-ionic detergent is a polyethylene type detergent.

82. The trimeric complex of claim 80, wherein the non-ionic detergent is a polyethylene type detergent.

83. The trimeric complex of claim 81, wherein the polyethylene type detergent is poly(oxyethylene)sorbitan monolaureate, poly(oxyethylene)sorbitan monooleate, or poly(oxyethylene)(20)sorbitan monolaureate.

84. The trimeric complex of claim 82, wherein the polyethylene type detergent is poly(oxyethylene)sorbitan monolaureate, poly(oxyethylene)sorbitan monooleate, or poly(oxyethylene)(20)sorbitan monolaureate.

85. A nucleic acid encoding a protein comprising
(A) (a) a consecutive amino acid sequence of a modified gp120 envelope polypeptide portion of a gp140 envelope of an HIV-1 KNH1144 isolate, or a quasi-species thereof; and (b) a consecutive amino acid sequence of a modified gp41 ectodomain polypeptide portion of the gp140 envelope of the HIV-1 KNH1144 isolate or such quasi-species thereof, the sequence of said modified gp120 envelope polypeptide portion and said modified gp41 ectodomain polypeptide portion of said HIV-1 KNH1144 isolate being as set forth in SEQ ID NO:2 and SEQ ID NO:3, respectively, said modified gp120 envelope polypeptide portion comprising a cysteine at amino acid position 511 and said modified gp41 ectodomain polypeptide portion comprising a cysteine at amino acid position 617 and a proline at amino acid position 571; wherein (i) the amino acid positions are numbered by reference to SEQ ID NO:1; (ii) the modified gp120 envelope polypeptide portion further comprises a mutated furin recognition sequence; and (iii) the modified gp120 polypeptide portion and the modified gp41 ectodomain polypeptide portion are bound to one another by a disulfide bond between the cysteine at amino acid position 511 and the cysteine at amino acid position 617; or
(B) (a) a consecutive amino acid sequence of a modified gp120 envelope polypeptide portion of a gp140 envelope of an HIV-1 5768.4 isolate, or a quasi-species thereof; and (b) a consecutive amino acid sequence of a modified gp41 ectodomain polypeptide portion of the gp140 envelope of the HIV-1 5768.4 isolate or such quasi-species thereof, the sequence of said modified gp120 envelope polypeptide portion and said modified gp41 ectodomain polypeptide portion of said HIV-1 5768.4 isolate being as set forth in SEQ ID NO:11 and SEQ ID NO:12, respectively, said modified gp120 envelope polypeptide portion comprising a cysteine at amino acid position 519 and said modified gp41 ectodomain polypeptide portion comprising a cysteine at amino acid position 625 and a proline at amino acid position 579, wherein (i) the amino acid positions are numbered by reference to SEQ ID NO:10, (ii) the modified gp120 envelope polypeptide portion further comprises a mutated furin recognition sequence, and (iii) the modified gp120 polypeptide portion and the modified gp41 ectodomain polypeptide portion are bound to one another by a disulfide bond between the cysteine at amino acid position 519 and the cysteine at amino acid position 625.

86. The nucleic acid of claim 85, wherein, in (A) or (B), the modified gp120 polypeptide portion is further characterized by (i) the absence of one or more canonical glycosylation sites present in wild-type HIV-1 gp120, (ii) the presence of one or more canonical glycosylation sites absent in wild-type HIV-1 gp120, or (iii) both (i) and (ii).

87. The nucleic acid of claim 85 which is DNA, cDNA or RNA.

88. A vector comprising the nucleic acid of claim 85.

89. A prokaryotic or eukaryotic host cell comprising the vector of claim 88.

90. A composition comprising the trimeric complex of claim 77.

91. A composition comprising the trimeric complex of claim 78.

92. The composition of claim 90 further comprising one or more of a pharmaceutically acceptable carrier, an adjuvant, or a non-ionic detergent.

93. The composition of claim 91 further comprising one or more of a pharmaceutically acceptable carrier, an adjuvant, or a non-ionic detergent.

94. A method of eliciting an immune response against HIV-1 or an HIV-1 infected cell in a subject, comprising administering to the subject a trimeric complex comprising three monomers, each of which is the protein of claim 73 (A) or (B), in an amount effective to elicit the immune response in the subject.

95. A method of eliciting an immune response against HIV-1 or an HIV-1 infected cell, comprising administering to the subject a trimeric complex comprising three monomers, each of which is the protein of claim 74 (A) or (B), in an amount effective to elicit the immune response in the subject.

96. The method of claim 94, wherein the trimeric complex is administered in a single dose, in multiple doses, or as part of a heterologous prime-boost regimen.

97. The method of claim 94, wherein the HIV-1 infected cell is present in a subject.

98. The method of claim 95, wherein the trimeric complex is administered in a single dose, in multiple doses, or as part of a heterologous prime-boost regimen.

99. The method of claim 95, wherein the HIV-1 infected cell is present in a subject.

100. A method of delaying the onset of, or slowing the rate of progression of, an HIV-1-related disease in an HIV-1-infected subject which comprises administering to the subject a trimeric complex comprising three monomers, each of which is the protein of claim 73 (A) or (B) in an amount effective to delay the onset of, or slowing the rate of progression of, the HIV-1-related disease in the subject.

101. A method of delaying the onset of, or slowing the rate of progression of, an HIV-1-related disease in an HIV-1-infected subject which comprises administering to the subject a trimeric complex comprising three monomers, each of which is the protein of claim 74 (A) or (B) in an amount effective to delay the onset of, or slowing the rate of progression of, the HIV-1-related disease in the subject.

102. The protein of claim 73, wherein, in (A), the quasi-species of the HIV-1 KNH1144 isolate comprises an HIV-1 viral isolate having a gp140 envelope sequence comprising less than or equal to 1% variation in sequence identity relative to SEQ ID NO:1; and in (B), the quasi-species of the HIV-1 5768.4 isolate comprises an HIV-1 viral isolate having a gp140 envelope sequence comprising less than or equal to 1% variation in sequence identity relative to SEQ ID NO:10.

103. The protein of claim 73, wherein, in (A), the mutated furin recognition sequence comprises amino acids 518 to 523 of SEQ ID NO:1; and in (B), the mutated furin recognition sequence comprises amino acids 526 to 531 of SEQ ID NO:10.

104. An isolated nucleic acid having the sequence as set forth in SEQ ID NO:13.

105. The isolated nucleic acid of claim 104, encoding a modified gp120 polypeptide portion and a modified gp41 ectodomain polypeptide portion of the gp140 envelope protein of an HIV-1 5768.4 isolate.


SEQUENCE LISTING (13 sequences)

SEQ ID NO: 1 - protein, 693 aa, Artificial Sequence
  modified human KNH1144 isolate
MDAMKRGLCCVLLLCGAVFVSPSQEIHARFRRGARNSNLWVTVYYGVPVWKDAETTLFCASDAKAYKTEKHNVWATHACVPTDPNPQEIPLENVTEEFNMWKNKMVEQMHTDIISLWDQSLQPCVKLAPLCVTLNCTDVTNVTDVSGTRGNITIMKEMEGEIKNCSFNMATEIRDKKQKVYSLFYRLDVVPINQGNSSSKNSSEYRLISCNTSAITQACPKVSFEPIPIHYCAPAGFAILKCRDKEFNGTGECKNVSTVQCTHGIKPVVSTQLLLNGSLAEEKVKIRTENITNNAKTIIVQLVEPVRISCTRPNNNTRESVRIGPGQAFFATGDIIGDIRQAHCNVSRSQWNKTLQQVAEQLREHFKNKTIIFNSSSGGDLEITTHSFNCGGEFFYCNTSGLFNSTWNTSMSGSSNTETNGTITLQCRIKQIINMWQRTGQAIYAPPIQGVIRCESNITGLLLTRDGGEEKNSTNEIFRPGGGDMRDNWRSELYKYKVVKIEPLGVAPTRCRRRVVGRRRRRRAVGIGAVFLGFLGAAGSTMGAASITLTVQARQLLSGIVQQQSNLLRAPEAQQHMLKLTVWGIKQLQARVLAVERYLRDQQLLGIWGCSGKLICCTNVPWNSSWSNKSQDEIWNNMTWLQWDKEISNYTNLIYSLIEESQNQQEKNGQDLLALGKWANLWTWFDISNWLWY

SEQ ID NO: 2 - protein, 488 aa, Artificial Sequence
  modified human gp120
NSNLWVTVYYGVPVWKDAETTLFCASDAKAYKTEKHNVWATHACVPTDPNPQEIPLENVTEEFNMWKNKMVEQMHTDIISLWDQSLQPCVKLAPLCVTLNCTDVTNVTDVSGTRGNITIMKEMEGEIKNCSFNMATEIRDKKQKVYSLFYRLDVVPINQGNSSSKNSSEYRLISCNTSAITQACPKVSFEPIPIHYCAPAGFAILKCRDKEFNGTGECKNVSTVQCTHGIKPVVSTQLLLNGSLAEEKVKIRTENITNNAKTIIVQLVEPVRISCTRPNNNTRESVRIGPGQAFFATGDIIGDIRQAHCNVSRSQWNKTLQQVAEQLREHFKNKTIIFNSSSGGDLEITTHSFNCGGEFFYCNTSGLFNSTWNTSMSGSSNTETNGTITLQCRIKQIINMWQRTGQAIYAPPIQGVIRCESNITGLLLTRDGGEEKNSTNEIFRPGGGDMRDNWRSELYKYKVVKIEPLGVAPTRCRRRVVGRRRRRR

SEQ ID NO: 3 - protein, 170 aa, Artificial Sequence
  modified human gp41
AVGIGAVFLGFLGAAGSTMGAASITLTVQARQLLSGIVQQQSNLLRAPEAQQHMLKLTVWGIKQLQARVLAVERYLRDQQLLGIWGCSGKLICCTNVPWNSSWSNKSQDEIWNNMTWLQWDKEISNYTNLIYSLIEESQNQQEKNGQDLLALGKWANLWTWFDISNWLWY

SEQ ID NO: 4 - protein, 6 aa, ARTIFICIAL SEQUENCE
  HIV EPITOPE
ALDKWS

SEQ ID NO: 5 - protein, 6 aa, ARTIFICIAL SEQUENCE
  HIV EPITOPE
ALGKWA

SEQ ID NO: 6 - protein, 6 aa, ARTIFICIAL SEQUENCE
  HIV EPITOPE
ALDKWA

SEQ ID NO: 7 - protein, 6 aa, ARTIFICIAL SEQUENCE
  HIV EPITOPE
ELDKWA

SEQ ID NO: 8 - protein, 9 aa, ARTIFICIAL SEQUENCE
  HIV EPITOPE
WFDISNWLW

SEQ ID NO: 9 - protein, 9 aa, ARTIFICIAL SEQUENCE
  HIV EPITOPE
WFDITKWLW

SEQ ID NO: 10 - protein, 700 aa, Artificial Sequence
  HIV modified 5768.4 gp140
LQAATMDAMKRGLCCVLLLCGAVFVSPSQEIHARFRRGARADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVITQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKAMYAPPIQGQIRCSSNITGLLLTRDGGGSNSSNTSEEVFRPGGGNMRDNWRSELYKYKVVKIEPLGIAPTKCKRRVVQRRRRRRTVGIGALFLGFLGAAGSTMGAASMTLTVQARQLLSGIVQQQNNLLRAPQAQQHLLQLTVWGIKQLQARVLAVERYLKDQQLLGIWGCSGKLICCTAVPWNASWSNKSLNEIWDNMTWMEWEKEIDNYTSLIYTLIEESQNQQEKNEQELLELDKWASLWNWFSITNWLWY

SEQ ID NO: 11 - protein, 490 aa, Artificial Sequence
  HIV 5768.4 modified gp120
ADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVITQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKAMYAPPIQGQIRCSSNITGLLLTRDGGGSNSSNTSEEVFRPGGGNMRDNWRSELYKYKVVKIEPLGIAPTKCKRRVVQRRRRRR

SEQ ID NO: 12 - protein, 170 aa, Artificial Sequence
  HIV 5768.4 modified gp41
TVGIGALFLGFLGAAGSTMGAASMTLTVQARQLLSGIVQQQNNLLRAPQAQQHLLQLTVWGIKQLQARVLAVERYLKDQQLLGIWGCSGKLICCTAVPWNASWSNKSLNEIWDNMTWMEWEKEIDNYTSLIYTLIEESQNQQEKNEQELLELDKWASLWNWFSITNWLWY

SEQ ID NO: 13 - DNA, 2176 bp, Artificial Sequence
  HIV 5687.4 modified gp140
ctgcaggccgccaccatggatgctatgaaacgaggattgtgttgtgttcttttgttgtgtggagctgtgtttgtgagtccaagtcaggagatacatgcgcggtttagaagaggggctcgcgcagataagctctgggttacagtgtattatggtgtgccagtgtggaaggagacaactaccactttgttttgcgcgtccgatgcgcgggcttatgacaccgaggtacacaatgtgtgggctacacatgcctgtgtccctaccgatcccaatcctcaggaagttgtactgggaaatgtcaccgaaaacttcaatatgtggaagaacaatatggtcgaacagatgcacgaggatattataagtctgtgggatcagtcactcaaaccctgtgtgcgcttgaccccattgtgtgtcacccttaactgcatcgactactatgggaatacaacaaactccaacaacagcagtgaaaccatgatggaaaagggtgaaattaagaactgttcttttaacataacaactagattgaaagataaaatgcagaaagaatatgccctgttttataaatacgacatagtccccattgacaatagggtgggaaacgacactagcaacgctacatcctatcgacttacttcttgcaatacgtctgtcattacccaggcctgccccaaggtttcatttgagcctatccccatccattactgcgccccagcaggattcgccatcctgaagtgcaacgacaagaaatttaacggcaccggcccatgcaagaatgtctccacggtgcagtgtacacatggtatcaagcccgtcgtgagcactcagcttcttcttaatggctctctggccgaggaagaggtcatgatccgatcagagaacttcacagacaatgcgaagacgattatcgtgcagctgaacgagacagtcgagatcaattgcactagaccaaacaataatacacggaagtcaatacacatgggacccgggaaggtgttctacacgactggcgagatcatcggcgatattcggcaagcccactgcaatattaatcgcgccaaatggaacaatacgctcatcaaaattgtggagaagttgcgggtgaagttcaacaagactatcagcttcaagcagtcttctggtggtgaccctgaaattgaaatgcactctttcaactgtggcggcgagttcttttactgcaacacaacccagctcttcaactctacctggtttaataacgccactctgaacgtgaacagtaatgttaccgagggaagtgagaacatcacactcccatgccggattaggcaaatagtgaatatgtggcaagaggtaggcaaggcaatgtacgcccctccgattcagggacaaattcggtgttcctccaacatcacaggcctgctgctgacccgagacggaggcggaagtaattcctccaacacaagtgaggaggtcttcaggcccggtggtgggaacatgcgagataattggaggtcagagctctacaagtataaggttgttaagattgagcctctcggtatcgctccgaccaagtgtaagcggagggtggtccaaaggagaagacggcgaagaaccgtcggaatcggtgccctgttcctgggcttcctgggcgcagccggctcaacaatgggtgctgcgagcatgactcttacggtacaggctcgccaacttcttagtggcatcgtgcaacaacagaataacctcttgagggcaccacaagcccagcaacacctgctgcaactgaccgtctggggcatcaagcaactgcaagcacgggtgctcgctgttgagagatacctgaaagaccagcagcttctcggtatttggggatgcagtggcaagctgatctgttgcactgccgtaccttggaacgcctcatggtccaataaaagtctcaatgaaatctgggacaatatgacttggatggagtgggaaaaggagattgacaattacacgtccctgatttatacccttatcgaagaatctcagaaccagcaggagaagaacgaacaggagctgctggagttggataaatgggcctccctgtggaattggtttagcataacgaattggttgtggtactgataagcggccgcttaatcgccatccagctgatattccctatagtgcatggtcatagctgtttcctggcagctct